(19) United States
(12) Patent Application Publication
(10) Pub. No.: US 2024/0294515 A1
(43) Pub. Date: Sep. 5, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Young Seok KIM, Daejeon (KR); Minjun KIM, Daejeon (KR); Dong Hoon LEE, Daejeon (KR); Sang Duk SUH, Daejeon (KR)

(21) Appl. No.: 18/568,483

(22) PCT Filed: Nov. 25, 2022
(86) PCT No.: PCT/KR2022/018846

§ 371 Date: Dec. 8, 2023

(30) Foreign Application Priority Data
Nov. 26, 2021 (KR) 10-2021-0166072
Nov. 24, 2022 (KR) 10-2022-0159473

(51) Int. Cl. C07D 405/14; C07D 409/14; H10K 85/60

(52) U.S. Cl. CPC C07D 405/14; C07D 409/14; H10K 85/654; H10K 85/6574; H10K 85/6576

(57) ABSTRACT

Provided is a compound of Chemical Formula 1:

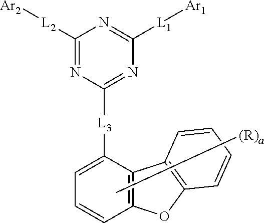

wherein: L₁ to L₃ are each independently a single bond or a substituted or unsubstituted C₆₋₆₀ arylene or C₂₋₆₀ heteroarylene containing at least one of N, O and S; Ar₁ and Ar₂ are each independently a substituted or unsubstituted C₆₋₆₀ aryl; or C₂₋₆₀ heteroaryl containing at least one of N, O and S, provided that Ar₁ and Ar₂ are not benzocarbazolyl, and at least one of Ar₁ and Ar₂ is an azachrysenyl, azabenzo[c]phenanthrenyl, azafluoranthenyl, azatetracenyl, azatetraphenyl, azapyrenyl, azatriphenylenyl, azaaceanthrylene, azaacephenanthrylene, or azapleiadenyl that is substituted or unsubstituted, and the other substituents are as defined in the specification; and an organic light emitting device comprising a first electrode, a second electrode, and one or more organic layers between the first electrode and the second electrode, wherein at least one layer among the organic layers contains the compound of Chemical Formula 1.

### CROSS-REFERENCE TO RELATED APPLICATION(S)

This application is a National Stage Application of International Application No. PCT/KR2022/018846 filed on Nov. 25, 2022, which claims priority to and the benefit of Korean Patent Application No. 10-2021-0166072 filed on Nov. 26, 2021 and Korean Patent Application No. 10-2022-0159473 filed on Nov. 24, 2022 in the Korean Intellectual Property Office, the entire contents of which are incorporated herein by reference.

### TECHNICAL FIELD

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### BRIEF DESCRIPTION

### Technical Problem

It is an object of the present disclosure to provide a novel compound and an organic light emitting device comprising the same.

### Technical Solution

According to an aspect of the present disclosure, provided is a compound of Chemical Formula 1:

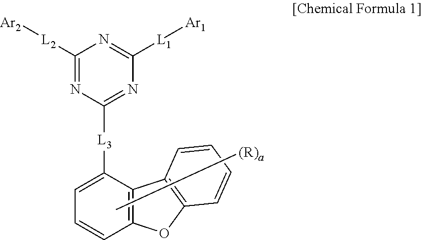

wherein, in Chemical Formula 1:
L₁ to L₃ are each independently a single bond, a substituted or unsubstituted C₆₋₆₀ arylene, or a substituted or unsubstituted C₂₋₆₀ heteroarylene containing at least one heteroatom of N, O and S;
Ar₁ and Ar₂ are each independently a substituted or unsubstituted C₆₋₆₀ aryl or a substituted or unsubstituted C₂₋₆₀ heteroaryl containing at least one heteroatom of N, O and S,
provided that Ar₁ and Ar₂ are not benzocarbazolyl, and at least one of Ar₁ and Ar₂ is a substituted or unsubstituted azachrysenyl, a substituted or unsubstituted azabenzo[c]phenanthrenyl, a substituted or unsubstituted azafluoranthenyl, a substituted or unsubstituted azatetracenyl, a substituted or unsubstituted azatetraphenyl, a substituted or unsubstituted azapyrenyl, a substituted or unsubstituted azatriphenylenyl, a substituted or unsubstituted azaaceanthrylene, a substituted or unsubstituted azaacephenanthrylene, or a substituted or unsubstituted azapleiadenyl;
R is deuterium or a substituted or unsubstituted C₆₋₆₀ aryl; and
a is an integer of 0 to 7.

According to another aspect of the present disclosure, provided is an organic light emitting device comprising: a first electrode; a second electrode that is opposite to the first electrode; and one or more organic material layers that are between the first electrode and the second electrode, wherein one or more layers of the one or more organic material layers comprises the compound of Chemical Formula 1.

### Advantageous Effects

The above-mentioned compound of Chemical Formula 1 can be used as a material of an organic material layer in an organic light emitting device, and can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a hole transport layer 3, a light emitting layer 4, an electron injection and transport layer 5 and a cathode 6.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6.

### DETAILED DESCRIPTION

Hereinafter, embodiments of the present disclosure will be described in more detail to facilitate understanding of the invention.

### Definition of Terms

As used herein, the notation [symbol] and [symbol] mean a bond linked to another substituent group.

As used herein, the term “substituted or unsubstituted” means being unsubstituted or substituted with one or more substituents selected from the group consisting of deuterium, a halogen group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl containing at least one of N, O and S atoms, or being unsubstituted or substituted with a substituent to which two or more substituents of the above-exemplified substituents are linked. For example, “a substituent in which two or more substituents are linked” can be a biphenyl group. Namely, a biphenylyl group can be an aryl group, or it can also be interpreted as a substituent in which two phenyl groups are linked. In one example, the term “substituted or unsubstituted” can be understood to mean “being unsubstituted or substituted with one or more, for example 1 to 5 substituents selected from the group consisting of deuterium, halogen, nitrile, a C₁₋₁₀ alkyl, a C₁₋₁₀ alkoxy and a C₆₋₂₀ aryl”. Further, the term “substituted with one or more substituents” can be understood to mean, for example, “being substituted with 1 to 5 substituents, or “being substituted with 1 to 2 substituents”.

In the present disclosure, the carbon number of a carbonyl group is not particularly limited, but is preferably 1 to 40. Specifically, it can be a substituent group having the following structure, but is not limited thereto:

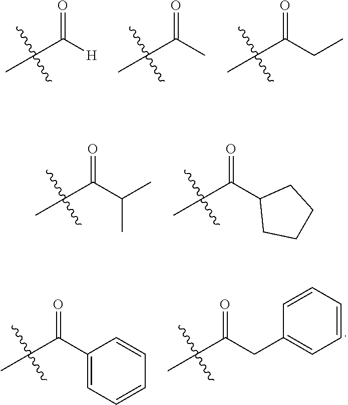

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, it can be a substituent group having the following structure, but is not limited thereto:

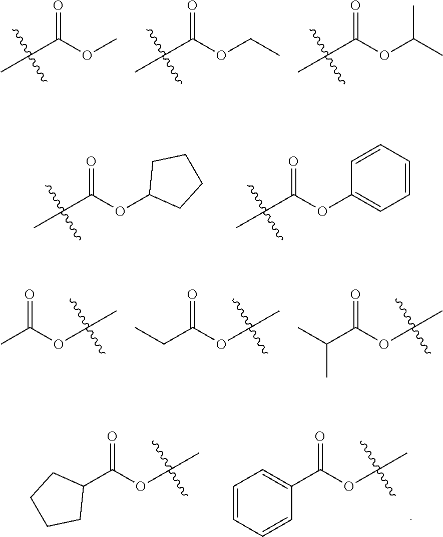

In the present disclosure, the carbon number of an imide group is not particularly limited, but is preferably 1 to 25. Specifically, it can be a substituent group having the following structure, but is not limited thereto:

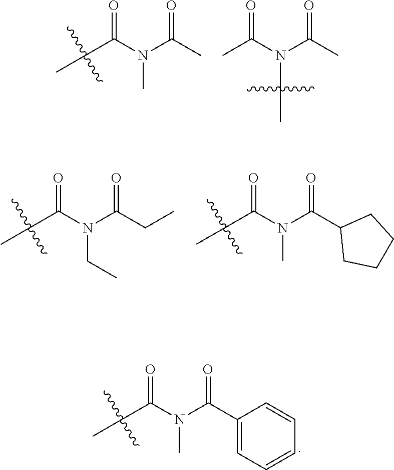

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, specific examples of the alkyl group include methyl, ethyl, propyl, n-propyl, isopropyl, butyl, n-butyl, isobutyl, tert-butyl, sec-butyl, 1-methyl-butyl, 1-ethylbutyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group having aromaticity. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthrenyl group, a triphenyleny, pyrenyl group, a perylenyl group, a chrysenyl group, and the like, but is not limited thereto.

In the present disclosure, a heteroaryl is a heteroaryl containing one or more of O, N, Si and S as a heteroatom, and the carbon number thereof is not particularly limited, but is preferably 2 to 60. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, the arylamine group and the arylsilyl group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine group can be applied to the aforementioned description of the heteroaryl. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl can be applied, except that the heterocycle is not a monovalent group but formed by combining two substituent groups.

### Compound

Meanwhile, the present disclosure provides the compound of Chemical Formula 1.

Specifically, the compound of Chemical Formula 1 has “1-dibenzofuranyl” and “a tetracyclic ring substituent group containing at least one N atom” as a substituent group of triazine. At this time, the tetracyclic ring substituent group containing at least one N atom means a substituted or unsubstituted azachrysenyl, a substituted or unsubstituted azabenzo[c]phenanthrenyl, a substituted or unsubstituted azafluoranthenyl, a substituted or unsubstituted azatetracenyl, a substituted or unsubstituted azatetraphenyl, a substituted or unsubstituted azapyrenyl, a substituted or unsubstituted azatriphenylenyl, a substituted or unsubstituted azaaceanthrylene, a substituted or unsubstituted azaacephenanthrylene, or a substituted or unsubstituted azapleiadenyl, provided that benzocarbazolyl is excluded.

As the compound of Chemical Formula 1 has one or more substituent groups selected from the group consisting of azachrysenyl, azabenzo[c]phenanthrenyl, azafluoranthenyl, azatetracenyl, azatetraphenyl, azapyrenyl, azatriphenylenyl, azaaceanthrylene, azaacephenanthrylenyl, and azapleiadenyl, while having no benzocarbazolyl, it can exhibit structural stability.

Accordingly, an organic light emitting device employing the above compound can not only exhibit a low driving voltage but also simultaneously improve efficiency and lifetime characteristics, as compared with the organic light emitting device that employs a compound having both 1-dibenzofuranyl and benzocarbazolyl and a compound having both 1-dibenzofuranyl and a tetracyclic ring substituent group containing at least one N atom having a structure different therefrom, respectively.

Wherein, “aza” described in front of the substituent group means a substituent group in which one or more CH in the substituent group is substituted with a nitrogen atom, “monoaza” refers to the case where one CH in a substituent group is substituted with a nitrogen atom, “diaza” refers to the case where two CH in a substituent group are substituted with nitrogen atoms, and “triaza” refers to the case where three CH in a substituent group are substituted with a nitrogen atom.

Therefore, “azachrysenyl” means a monovalent substituent of a compound in which at least one CH in chrysene is substituted with nitrogen atoms, “monoazachrysenyl” is selected from monovalent substituent groups of the following compounds, and the IUPAC NAME of the following compounds is the same as the name listed under the compound.

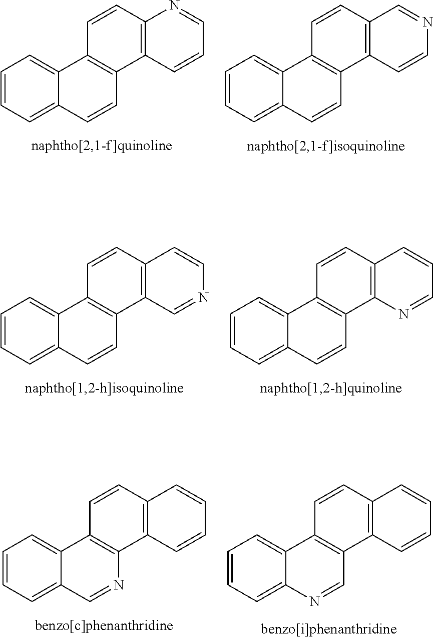

The structures of azabenzo[c]phenanthrenyl, azafluoranthenyl, azatetracenyl, azatetraphenyl, azapyrenyl, azatriphenylenyl, azaaceanthrylene, azaacephenanthrylenyl, and azapleiadenyl can also be understood with reference to the description of “azachrysenyl”. In addition, chrysene, benzo[c]phenanthrene, fluoranthene, tetracene, tetraphene, pyrene, triphenylene, aceanthrylene, acephenanthrylene and pleiadene mean the structures show below, respectively:

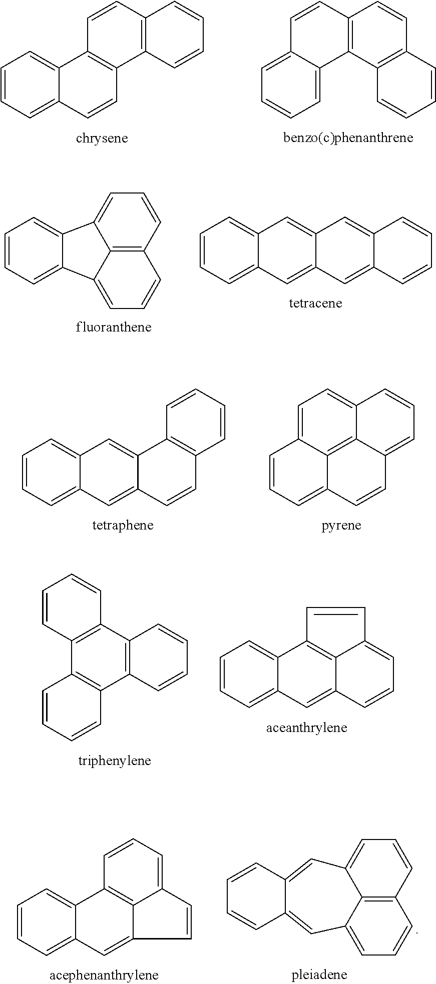

In one embodiment, L₁ and L₂ can be each independently a single bond or a C₆₋₂₀ arylene that is unsubstituted or substituted with deuterium.

Specifically, L₁ and L₂ can be each independently a single bond or phenylene. When L₁ and L₂ are each independently phenylene, they can be unsubstituted or substituted with at least one deuterium.

At this time, L₁ and L₂ can be the same as each other. Alternatively, L₁ and L₂ can be different.

For example, both L₁ and L₂ are single bonds; or
L₁ is phenylene and L₂ can be a single bond.

When L₁ is phenylene, it can be unsubstituted or substituted with at least one deuterium.

Further, L₃ can be a single bond or a C₆₋₂₀ arylene that is unsubstituted or substituted with deuterium.

Specifically, L₃ can be a single bond, phenylene, or naphthylene.

When L₃ is phenylene or naphthylene, it can be unsubstituted or substituted with at least one deuterium.

For example, L₃ can be a single bond or any one selected from the group consisting of:

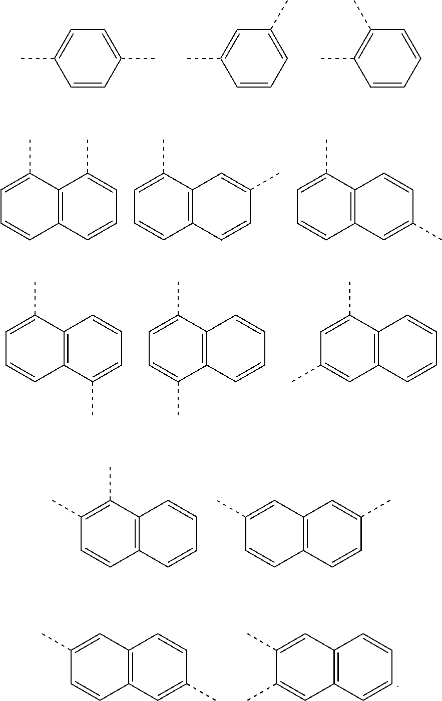

When L₃ is selected from the above group, it can be unsubstituted or substituted with deuterium.

Preferably, L₃ is a single bond, or any one selected from the group consisting of:

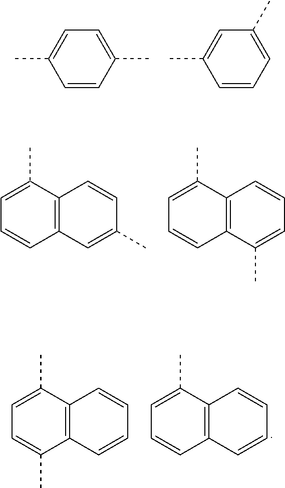

When L₃ is selected from the above group, it can be unsubstituted or substituted with deuterium.

Further, Ar₁ is monoazachrysenyl, monoazabenzo[c]phenanthrenyl, monoazafluoranthenyl, monoazatetracenyl, monoazatetraphenyl, monoazapyrenyl, monoazatriphenylenyl, monoazaaceanthrylene, monoazaacephenanthrylenyl, or monoazapleiadenyl,
Ar₂ is a C₆₋₂₀ aryl or a C₂₋₂₀ heteroaryl containing at least one heteroatom of O and S, and
Ar₁ and Ar₂ can be unsubstituted or substituted with at least one deuterium.

Specifically, Ar₁ can be any one selected from the group consisting of the following:

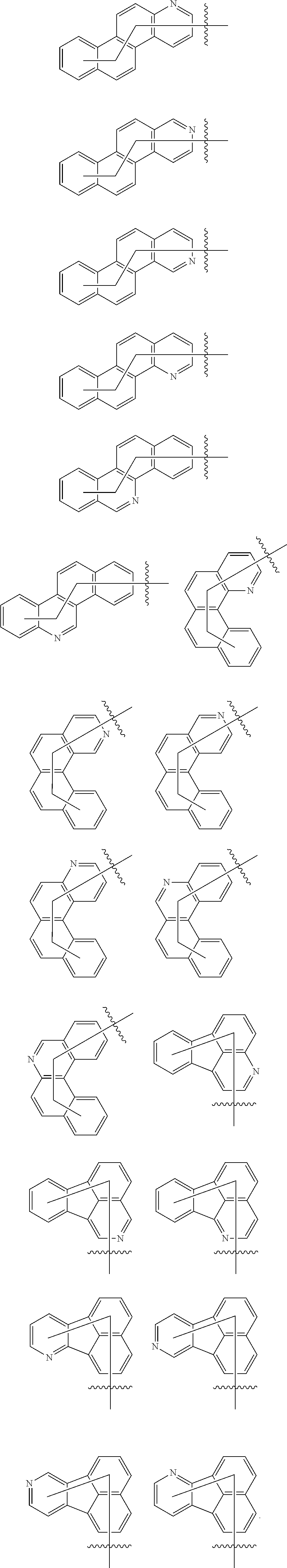

When Ar₁ is selected from the group consisting of the above, it can be unsubstituted or substituted with at least one deuterium.

And, Ar₂ can be phenyl, biphenylyl, naphthyl, dibenzofuranyl, or dibenzothiophenyl.

Ar₂ can be unsubstituted or substituted with at least one deuterium.

For example, Ar₂ can be any one selected from the group consisting of:

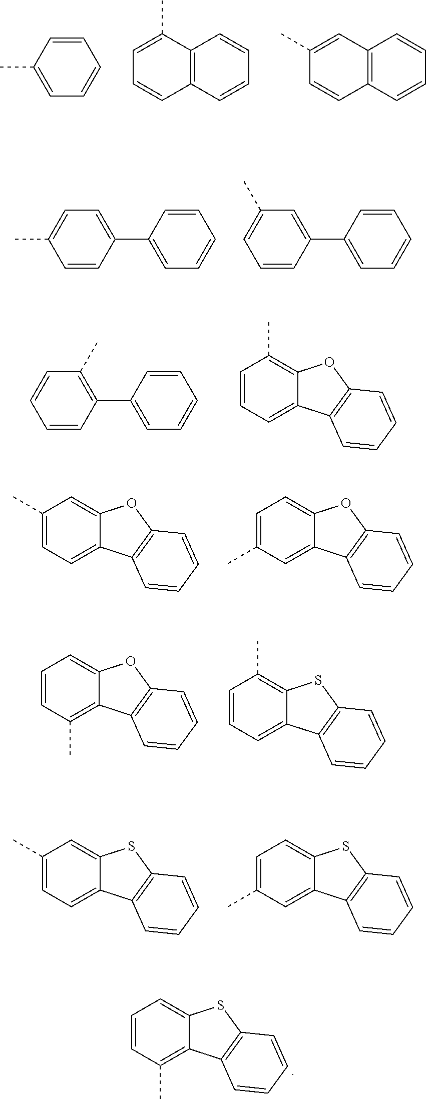

When Ar₂ is selected from the group consisting of the above, it can be unsubstituted or substituted with at least one deuterium.

Here, Ar₁ and Ar₂ can be different.

Further, L₁-Ar₁ and L₂-Ar₂ can be different.

Further, L₁-Ar₁, L₂-Ar₂ and

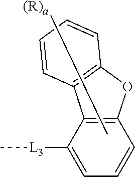

all can be different.

In addition, R can be deuterium, or a C₆₋₂₀ aryl unsubstituted or substituted with at least one deuterium.

And, a means the number of R, and a is 0, 1, 2, 3, 4, 5, 6, or 7.

As an example, R is phenyl, and a can be 0 or 1.

When R is phenyl, it can be unsubstituted or substituted with at least one deuterium.

Further, the compound can be any one of the following Chemical Formulas 1-1 to 1-3:

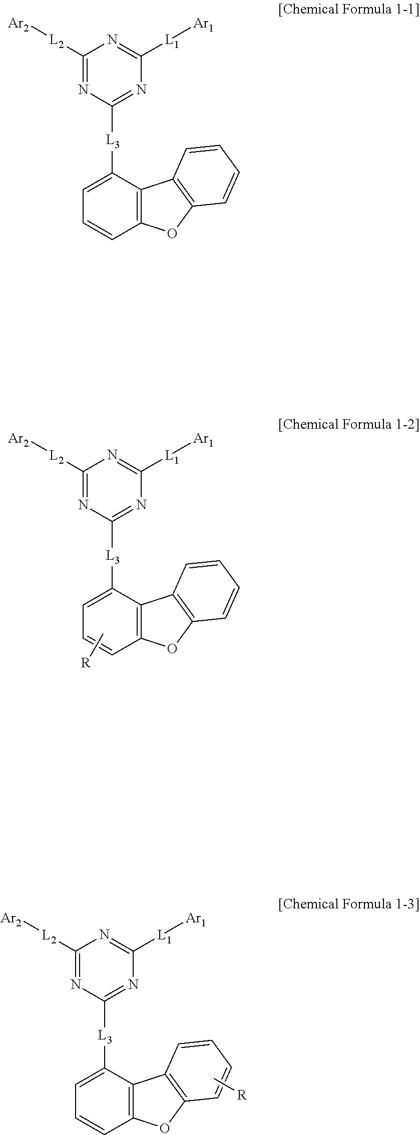

wherein, in Chemical Formulas 1-1 to 1-3:
R is a C₆₋₂₀ aryl that is unsubstituted or substituted with at least one deuterium; and
L₁ to L₃, Ar₁ and Ar₂ are as defined in Chemical Formula 1.

Meanwhile, representative examples of the compound of Chemical Formula 1 areas follows:

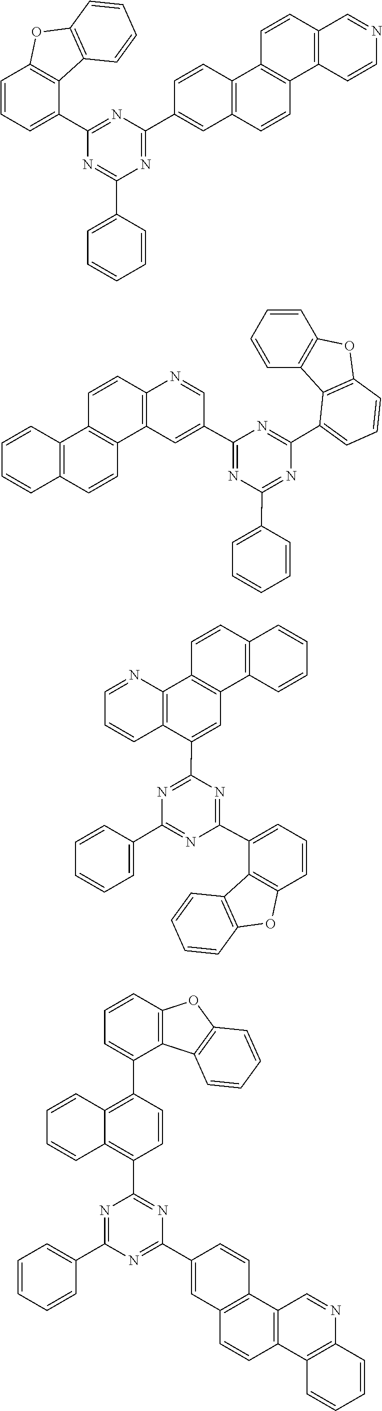

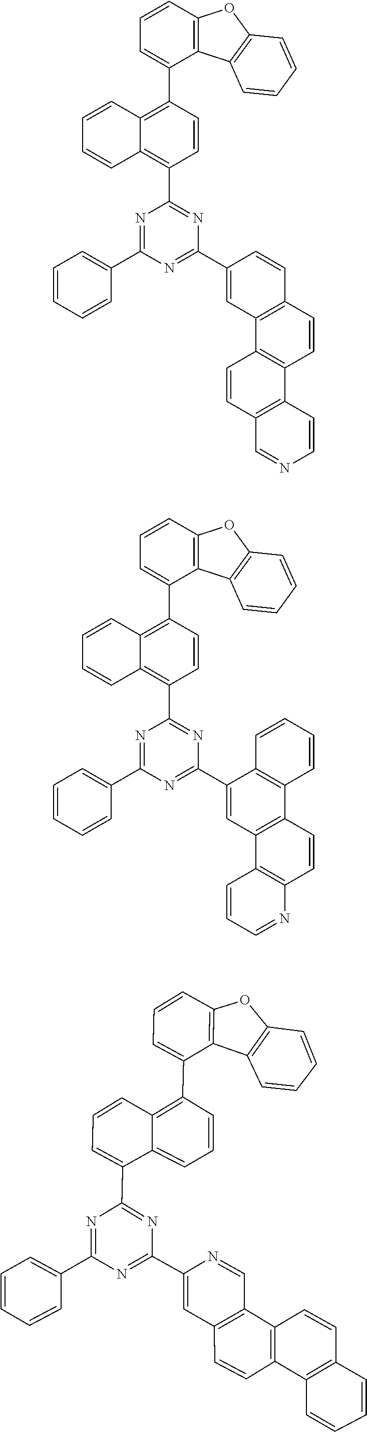 [SMILES: c1ccc(-c2nc(-c3ccc4ccc5c6ccncc6ccc5c4c3)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cc4c5cccnc5ccc4c4ccccc34)n2)cc1 ; c1ccc(-c2nc(-c3cc4ccc5c6ccccc6ccc5c4cn3)nc(-c3cccc4c(-c5cccc6oc7ccccc7c56)cccc34)n2)cc1]

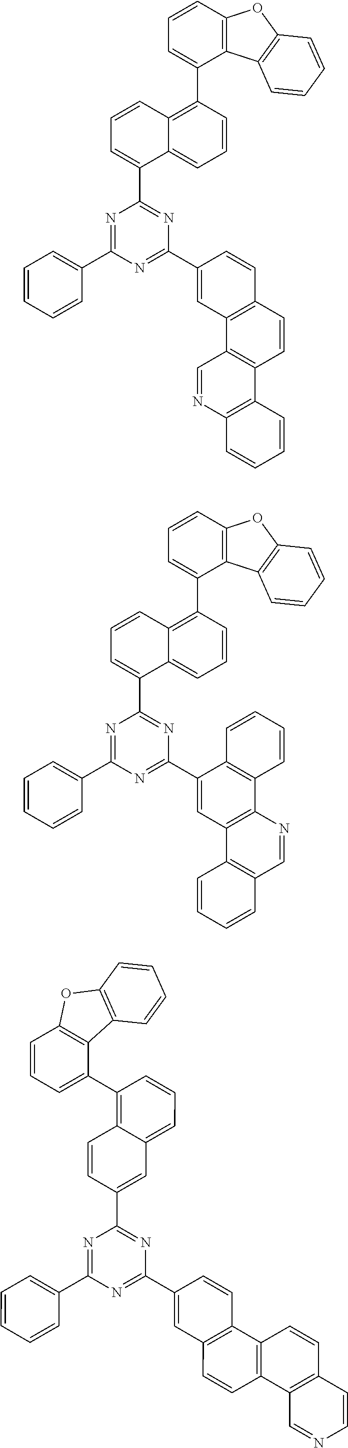 [SMILES: c1ccc(-c2nc(-c3ccc4ccc5c6ccccc6ncc5c4c3)nc(-c3cccc4c(-c5cccc6oc7ccccc7c56)cccc34)n2)cc1 ; c1ccc(-c2nc(-c3cccc4c(-c5cccc6oc7ccccc7c56)cccc34)nc(-c3cc4c5ccccc5cnc4c4ccccc34)n2)cc1 ; c1ccc(-c2nc(-c3ccc4c(-c5cccc6oc7ccccc7c56)cccc4c3)nc(-c3ccc4c(ccc5c6cnccc6ccc45)c3)n2)cc1]

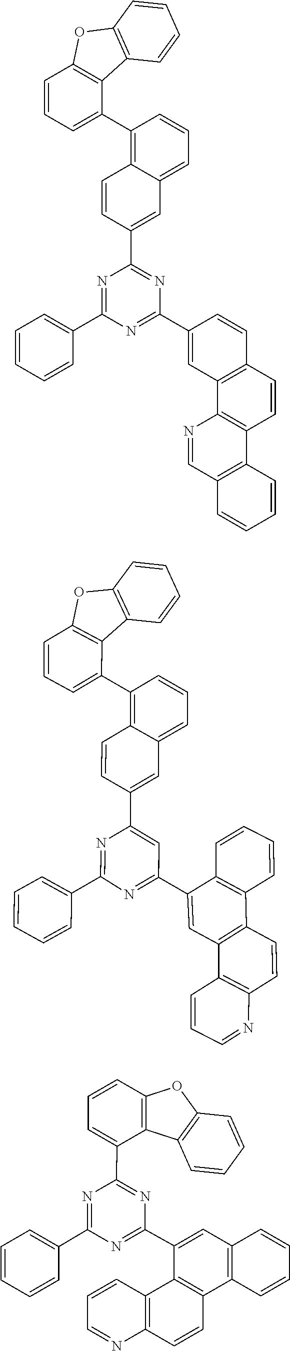 [SMILES: c1ccc(-c2nc(-c3ccc4c(-c5cccc6oc7ccccc7c56)cccc4c3)nc(-c3ccc4ccc5c6ccccc6cnc5c4c3)n2)cc1 ; c1ccc(-c2nc(-c3ccc4c(-c5cccc6oc7ccccc7c56)cccc4c3)cc(-c3cc4c5cccnc5ccc4c4ccccc34)n2)cc1 ; c1ccc(-c2nc(-c3cccc4oc5ccccc5c34)nc(-c3cc4ccccc4c4ccc5ncccc5c34)n2)cc1]

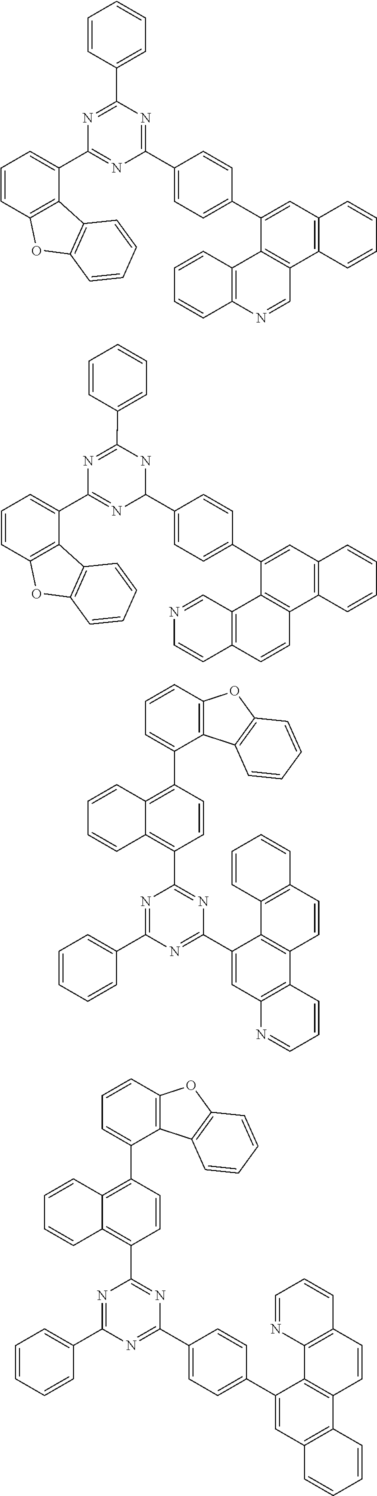

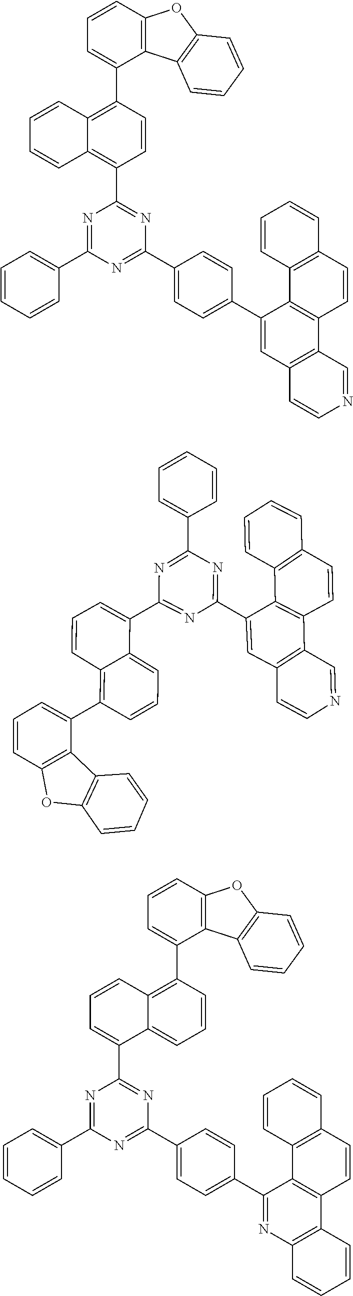 [SMILES: c1ccc(-c2nc(-c3ccc(-c4cc5ccncc5c5ccc6ccccc6c45)cc3)nc(-c3ccc(-c4cccc5oc6ccccc6c45)c4ccccc34)n2)cc1 ; c1ccc(-c2nc(-c3cccc4c(-c5cccc6oc7ccccc7c56)cccc34)nc(-c3cc4ccncc4c4ccc5ccccc5c34)n2)cc1 ; c1ccc(-c2nc(-c3ccc(-c4nc5ccccc5c5ccc6ccccc6c45)cc3)nc(-c3cccc4c(-c5cccc6oc7ccccc7c56)cccc34)n2)cc1]

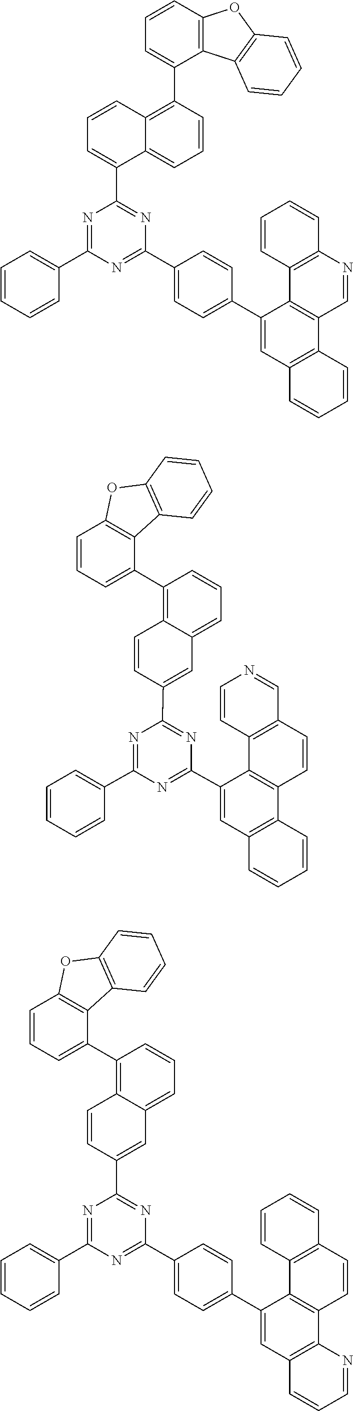 [SMILES: c1ccc(-c2nc(-c3ccc(-c4cc5ccccc5c5cnc6ccccc6c45)cc3)nc(-c3cccc4c(-c5cccc6oc7ccccc7c56)cccc34)n2)cc1 ; c1ccc(-c2nc(-c3ccc4c(-c5cccc6oc7ccccc7c56)cccc4c3)nc(-c3cc4ccccc4c4ccc5cnccc5c34)n2)cc1 ; c1ccc(-c2nc(-c3ccc(-c4cc5cccnc5c5ccc6ccccc6c45)cc3)nc(-c3ccc4c(-c5cccc6oc7ccccc7c56)cccc4c3)n2)cc1]

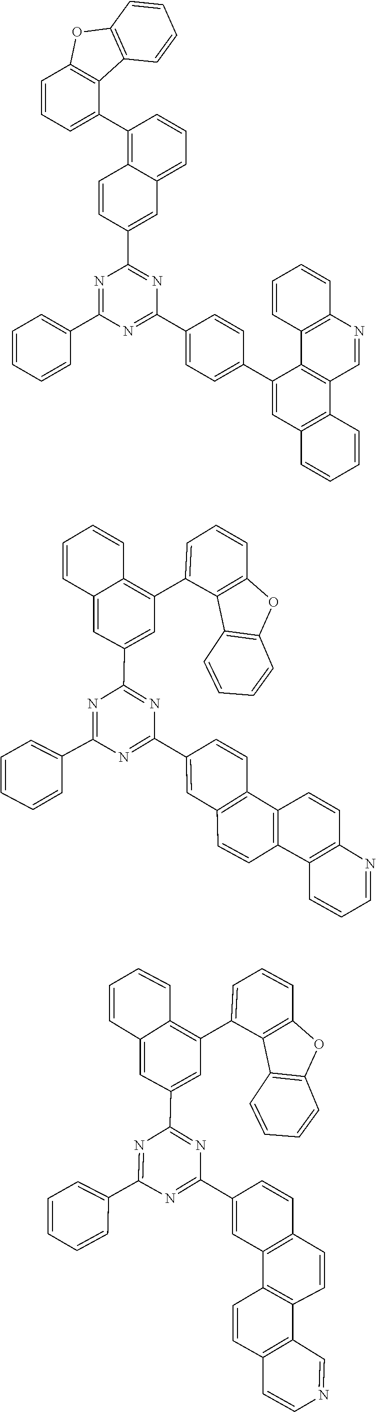 [SMILES: c1ccc(-c2nc(-c3ccc(-c4cc5ccccc5c5cnc6ccccc6c45)cc3)nc(-c3ccc4c(-c5cccc6oc7ccccc7c56)cccc4c3)n2)cc1 ; c1ccc(-c2nc(-c3cc(-c4cccc5oc6ccccc6c45)c4ccccc4c3)nc(-c3ccc4c(ccc5c6cccnc6ccc45)c3)n2)cc1 ; c1ccc(-c2nc(-c3cc(-c4cccc5oc6ccccc6c45)c4ccccc4c3)nc(-c3ccc4ccc5c6cnccc6ccc5c4c3)n2)cc1]

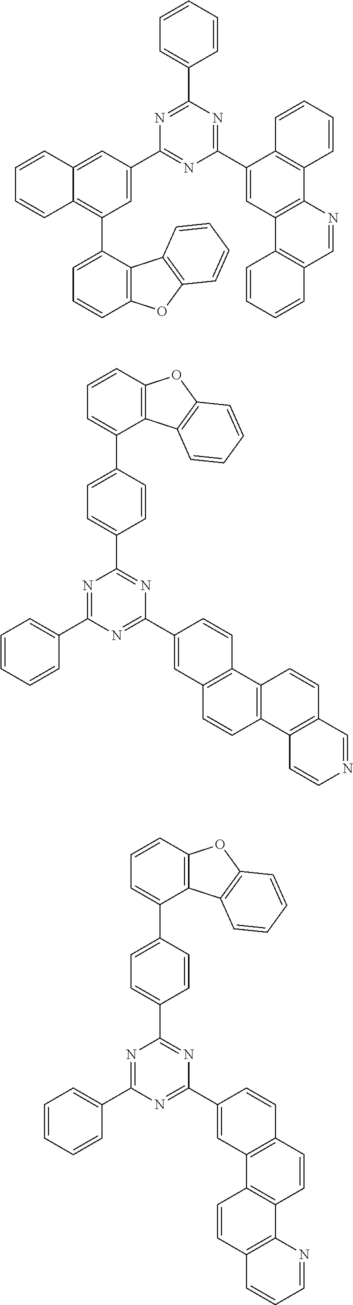 [SMILES: c1ccc(-c2nc(-c3cc(-c4cccc5oc6ccccc6c45)c4ccccc4c3)nc(-c3cc4c5ccccc5cnc4c4ccccc34)n2)cc1 ; c1ccc(-c2nc(-c3ccc(-c4cccc5oc6ccccc6c45)cc3)nc(-c3ccc4c(ccc5c6ccncc6ccc45)c3)n2)cc1 ; c1ccc(-c2nc(-c3ccc(-c4cccc5oc6ccccc6c45)cc3)nc(-c3ccc4ccc5c(ccc6cccnc65)c4c3)n2)cc1]

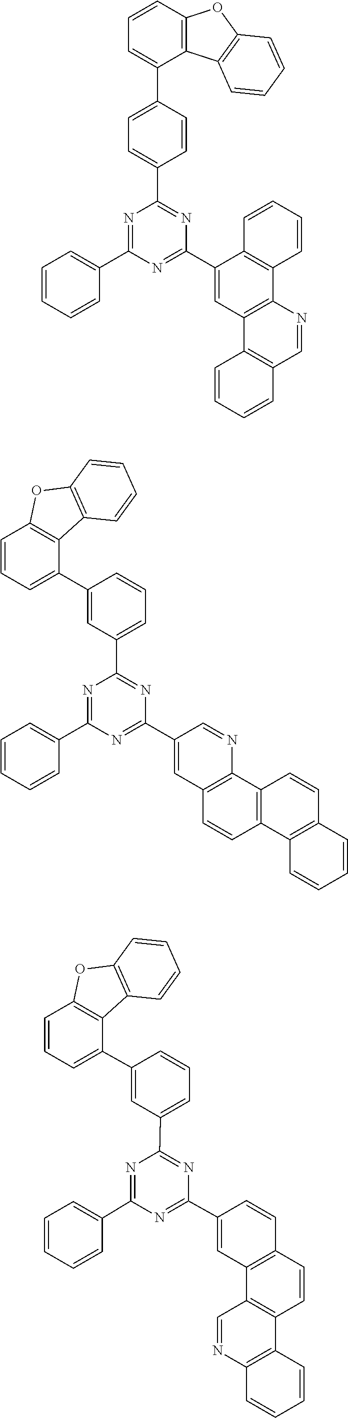 [SMILES: c1ccc(-c2nc(-c3ccc(-c4cccc5oc6ccccc6c45)cc3)nc(-c3cc4c5ccccc5cnc4c4ccccc34)n2)cc1 ; c1ccc(-c2nc(-c3cccc(-c4cccc5oc6ccccc6c45)c3)nc(-c3cnc4c(ccc5c6ccccc6ccc54)c3)n2)cc1 ; c1ccc(-c2nc(-c3cccc(-c4cccc5oc6ccccc6c45)c3)nc(-c3ccc4ccc5c6ccccc6ncc5c4c3)n2)cc1]

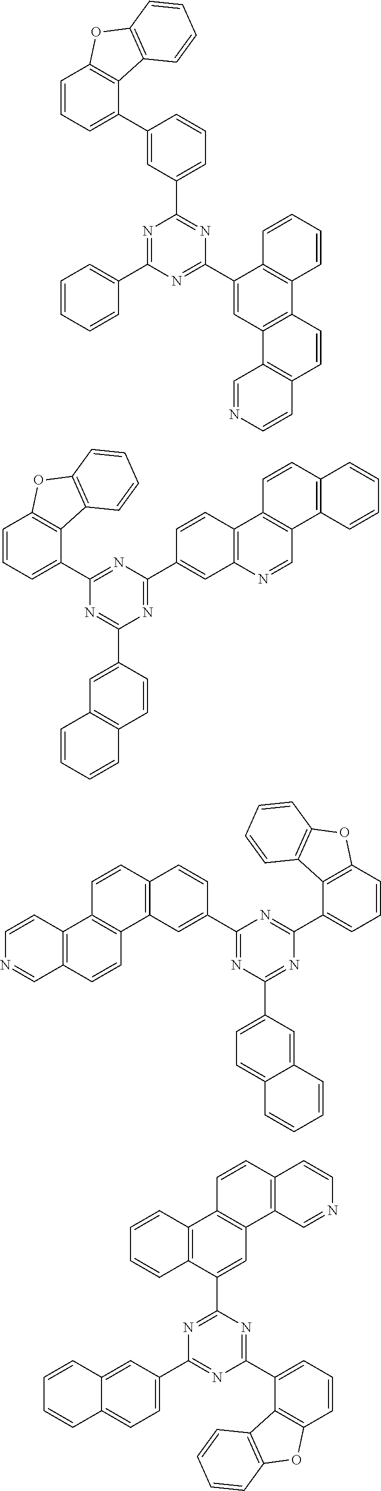

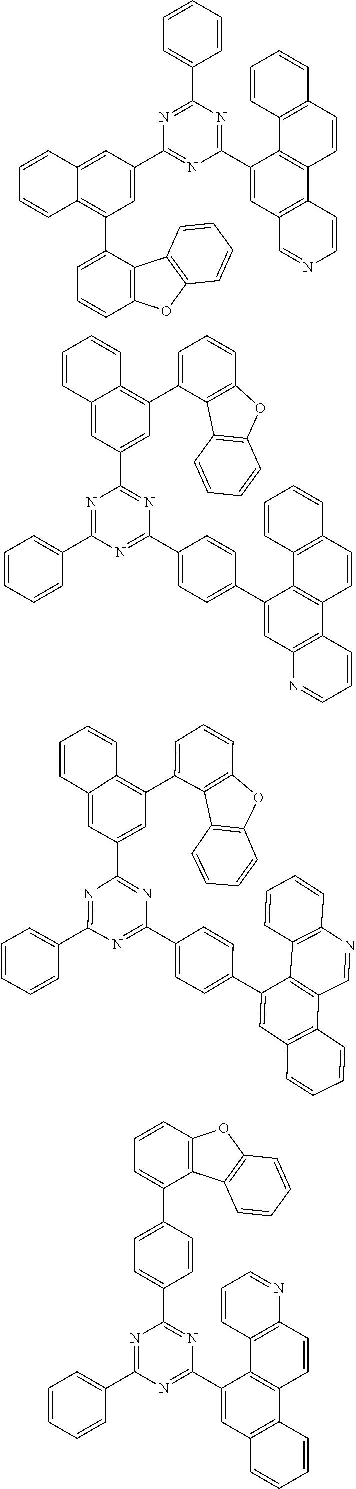

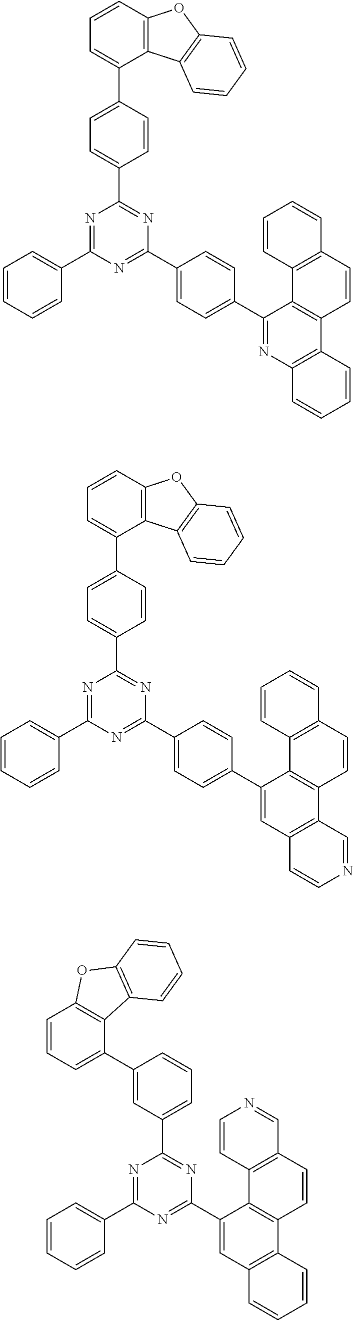 [SMILES: c1ccc(-c2nc(-c3ccc(-c4cccc5oc6ccccc6c45)cc3)nc(-c3ccc(-c4nc5ccccc5c5ccc6ccccc6c45)cc3)n2)cc1 ; c1ccc(-c2nc(-c3ccc(-c4cccc5oc6ccccc6c45)cc3)nc(-c3ccc(-c4cc5ccncc5c5ccc6ccccc6c45)cc3)n2)cc1 ; c1ccc(-c2nc(-c3cccc(-c4cccc5oc6ccccc6c45)c3)nc(-c3cc4ccccc4c4ccc5cnccc5c34)n2)cc1]

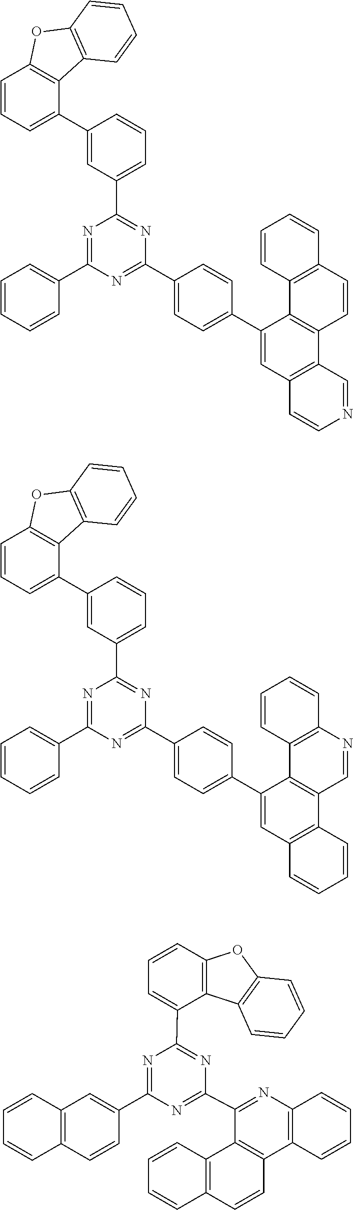 [SMILES: c1ccc(-c2nc(-c3ccc(-c4cc5ccncc5c5ccc6ccccc6c45)cc3)nc(-c3cccc(-c4cccc5oc6ccccc6c45)c3)n2)cc1 ; c1ccc(-c2nc(-c3ccc(-c4cc5ccccc5c5cnc6ccccc6c45)cc3)nc(-c3cccc(-c4cccc5oc6ccccc6c45)c3)n2)cc1 ; c1ccc2cc(-c3nc(-c4cccc5oc6ccccc6c45)nc(-c4nc5ccccc5c5ccc6ccccc6c45)n3)ccc2c1]

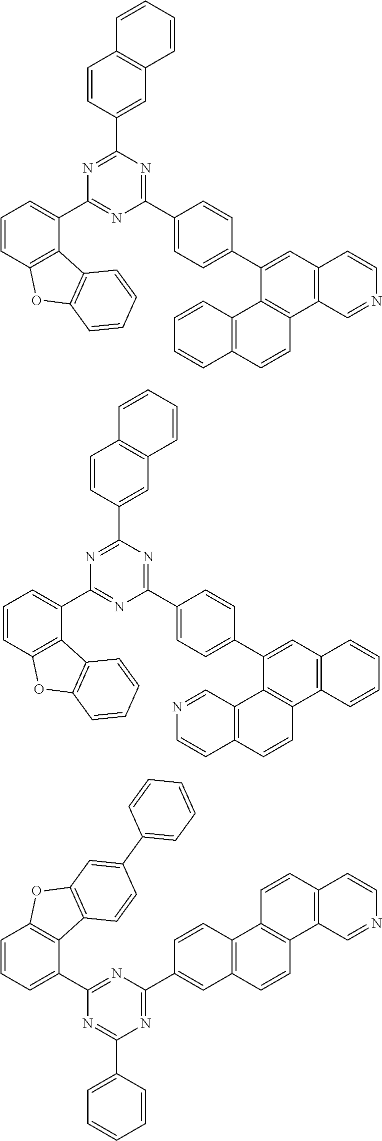

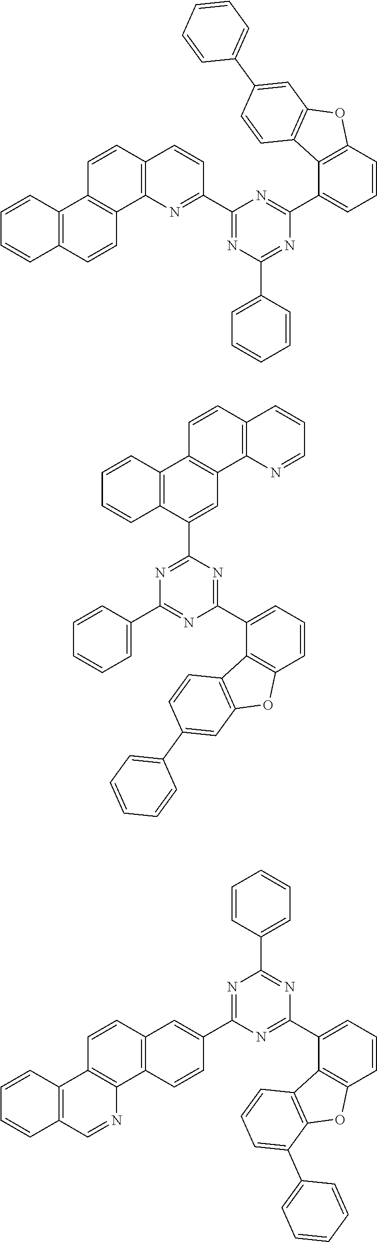

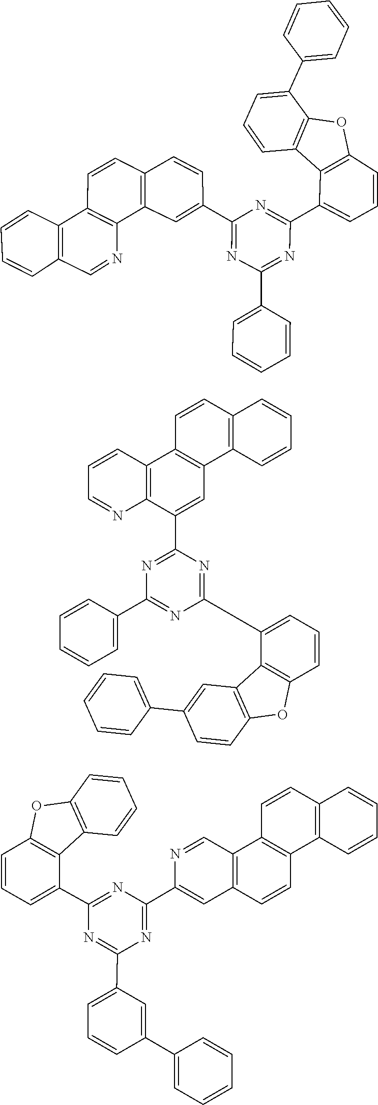

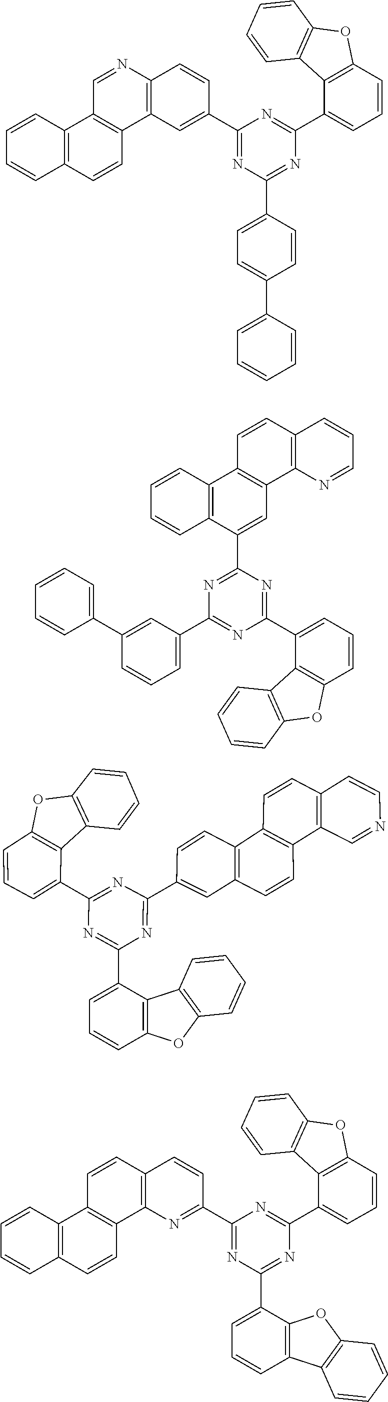

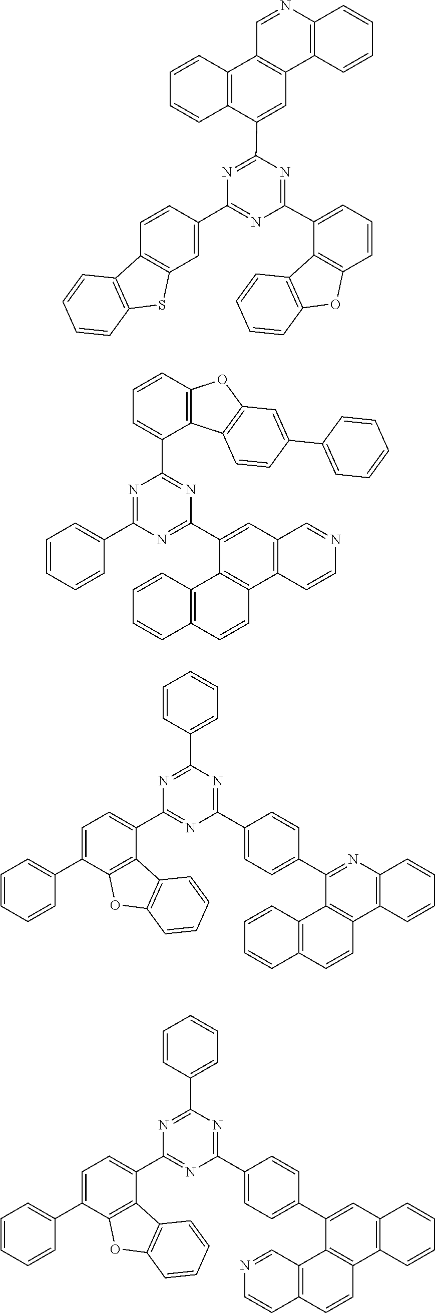

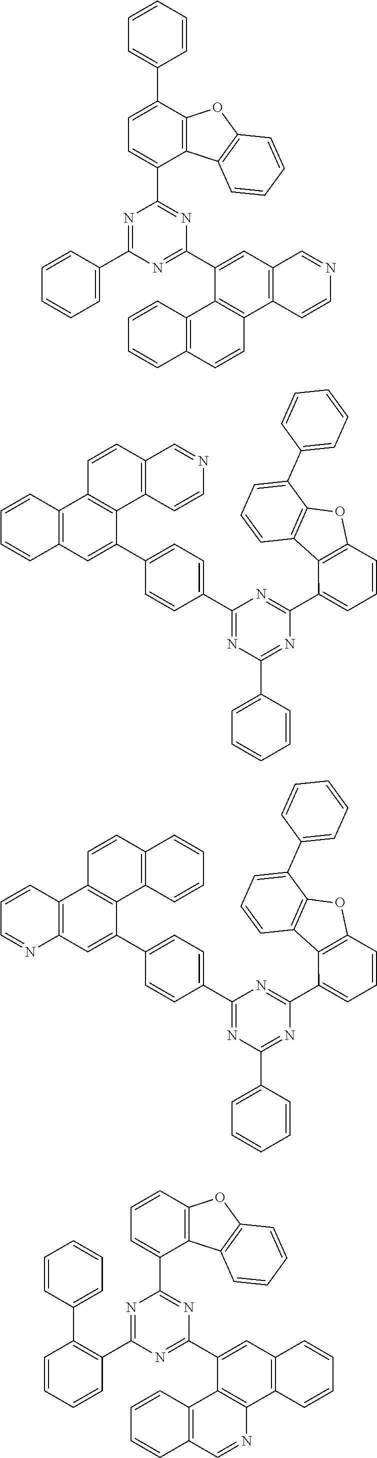

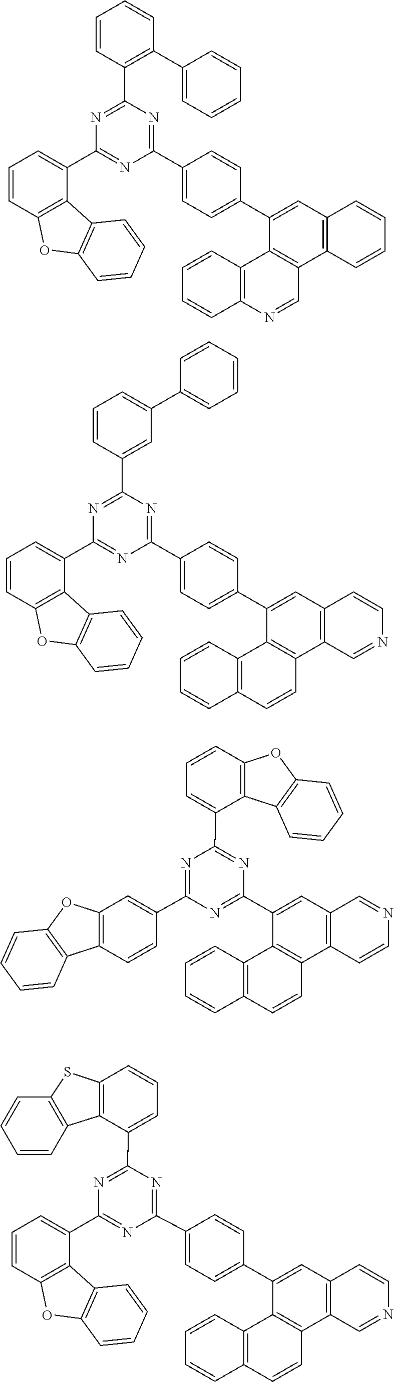

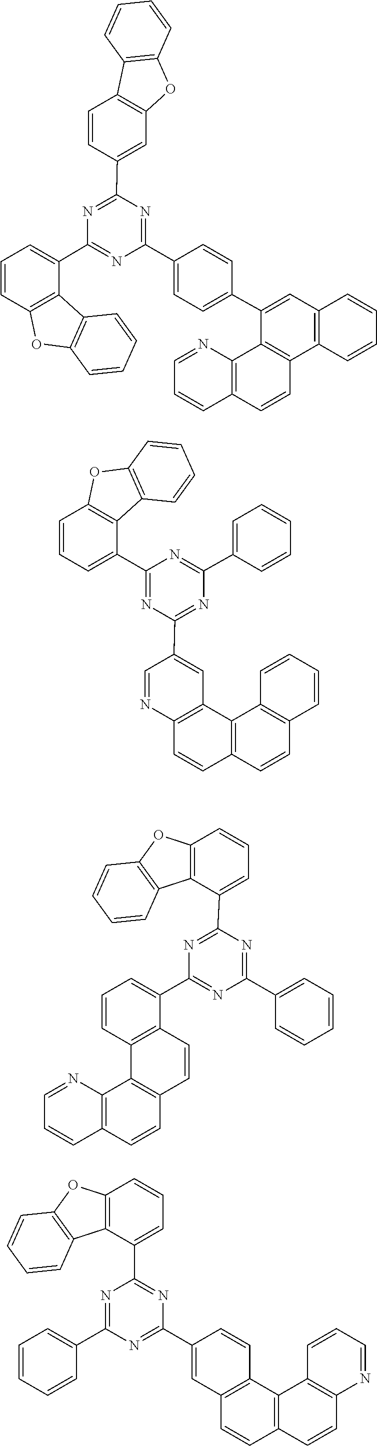

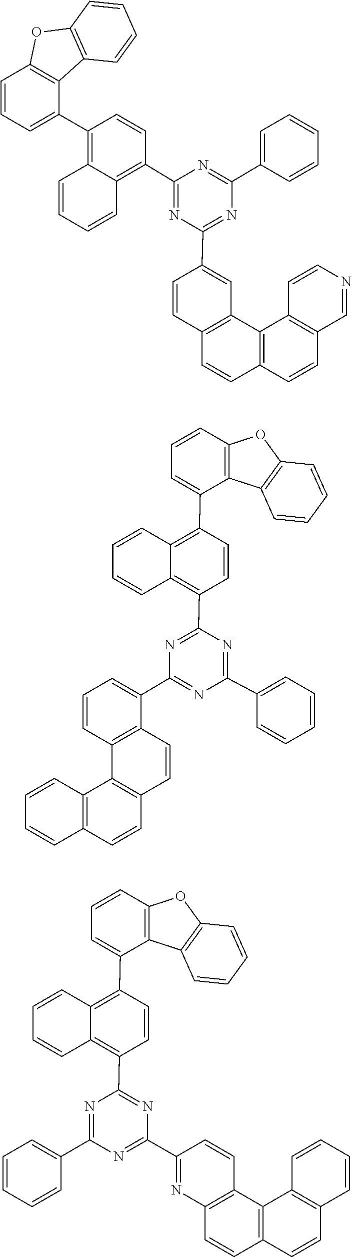

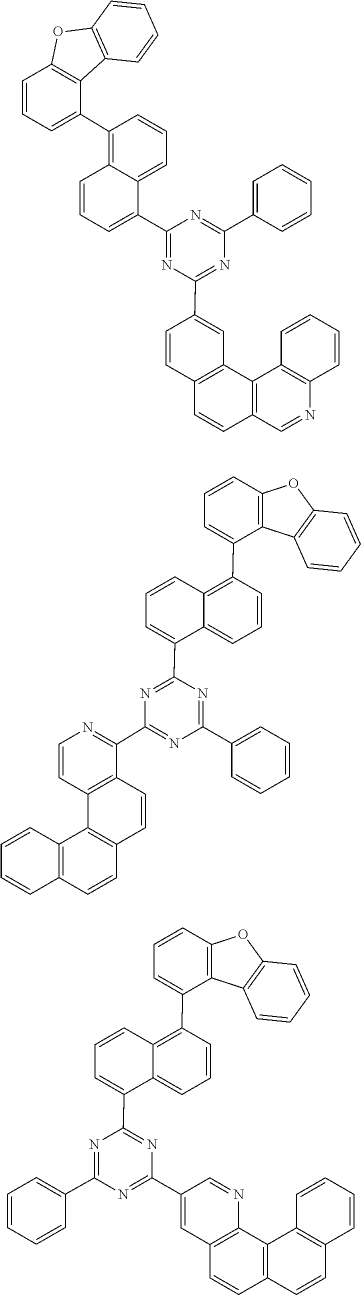

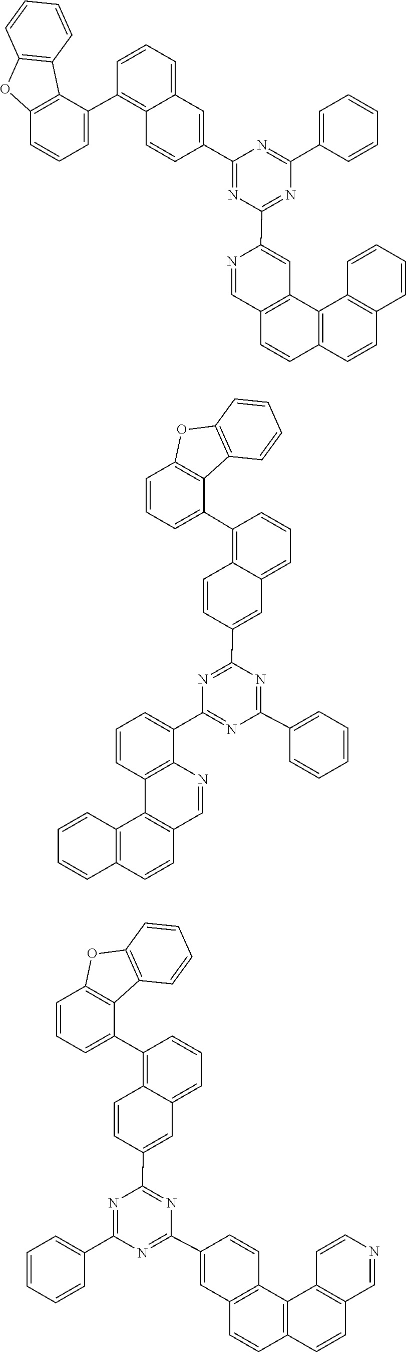

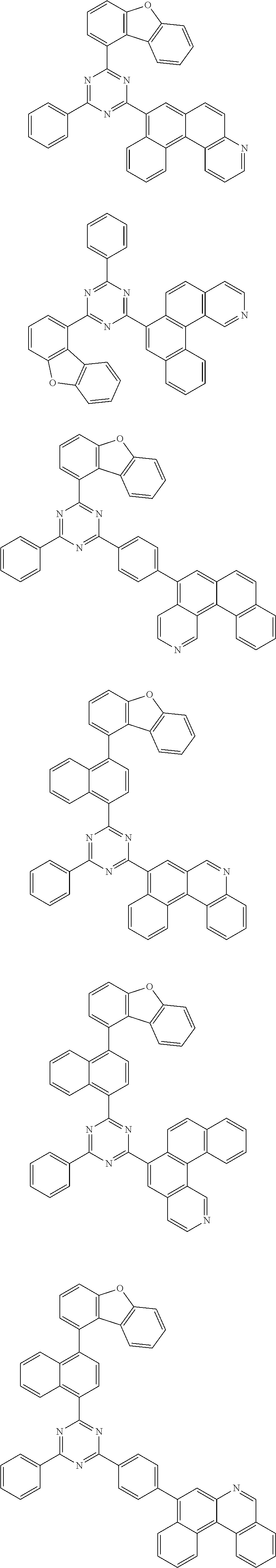

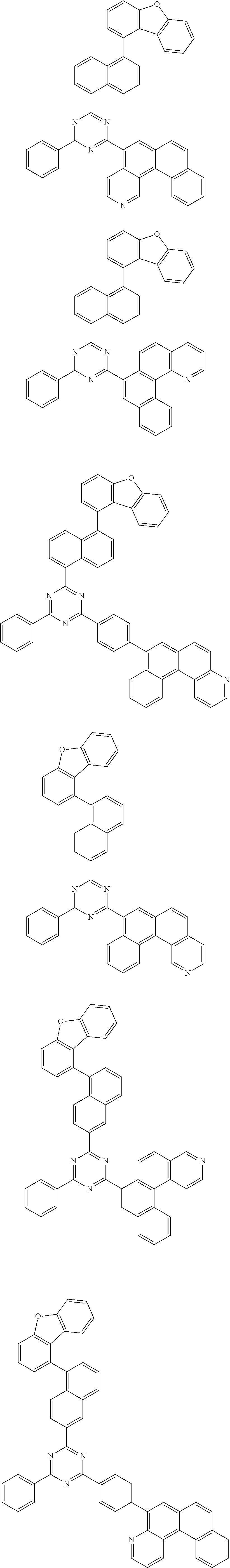

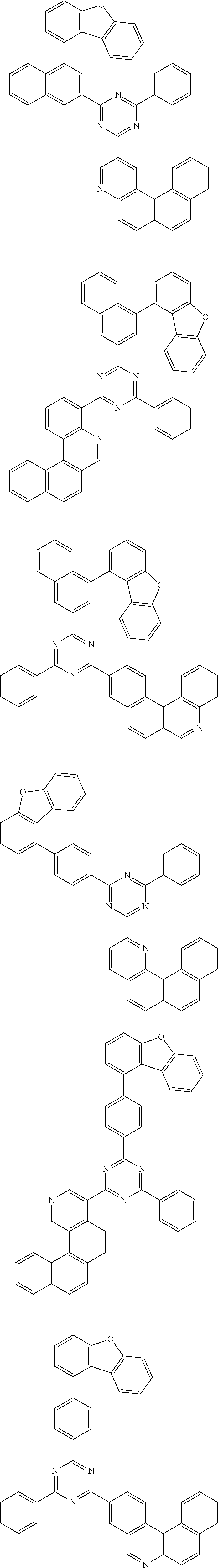

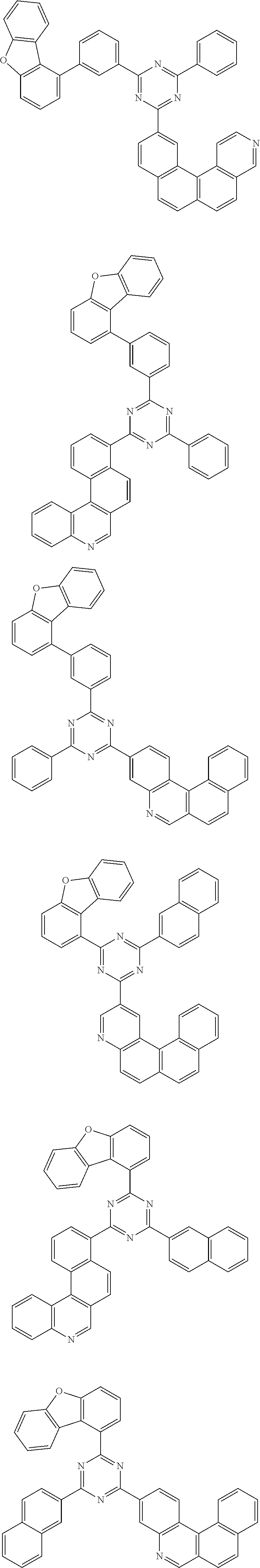

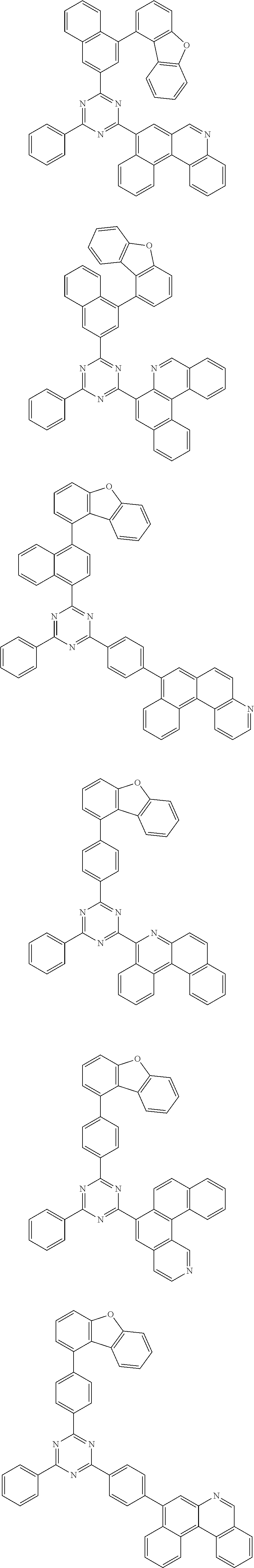

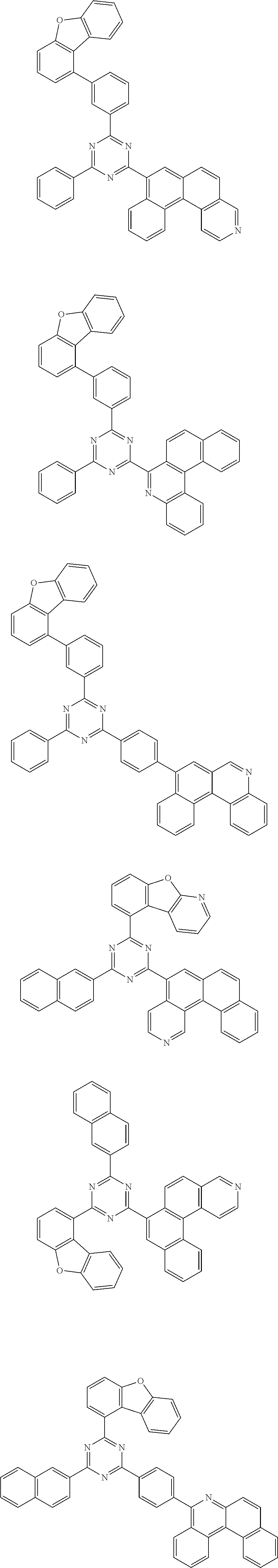

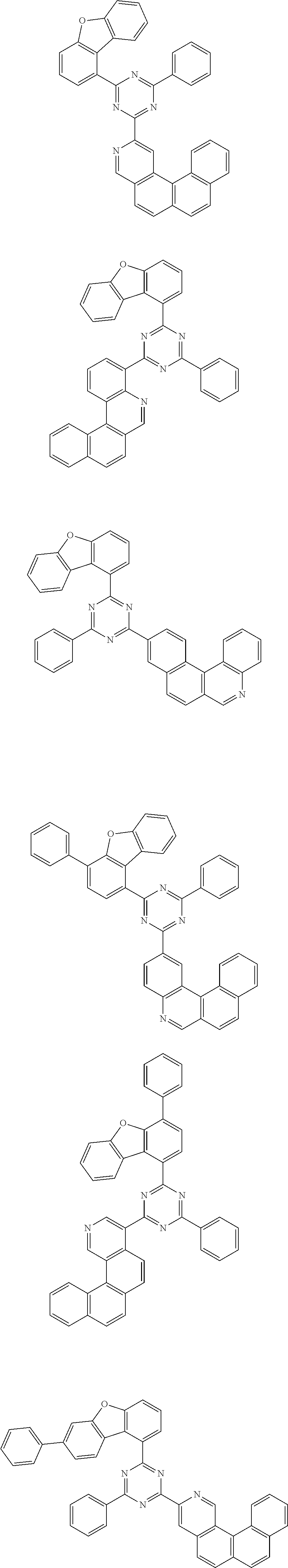

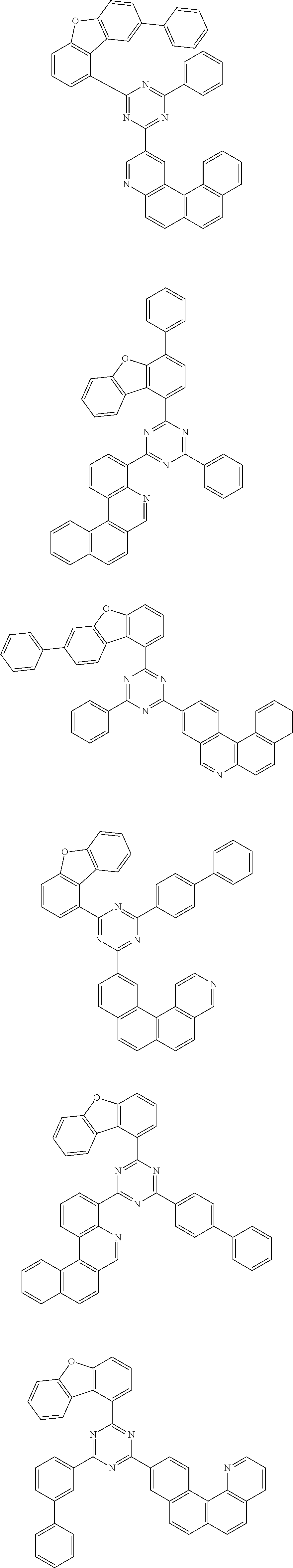

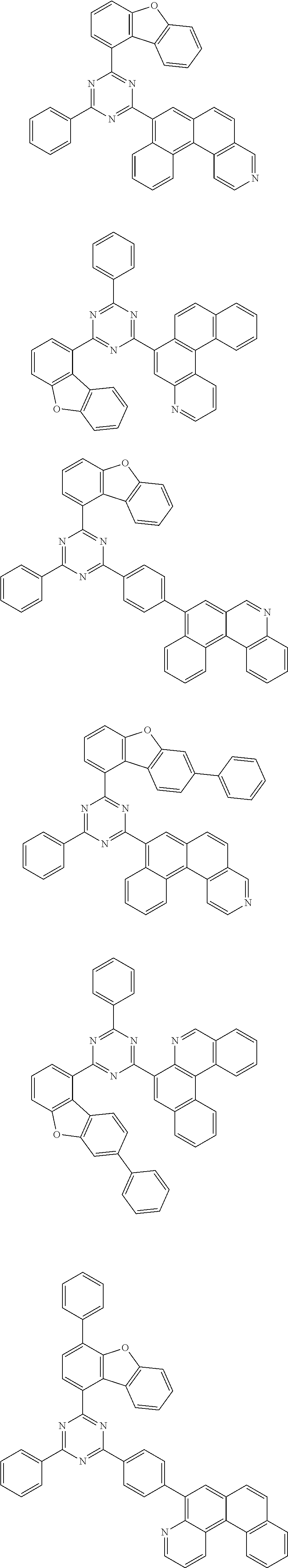

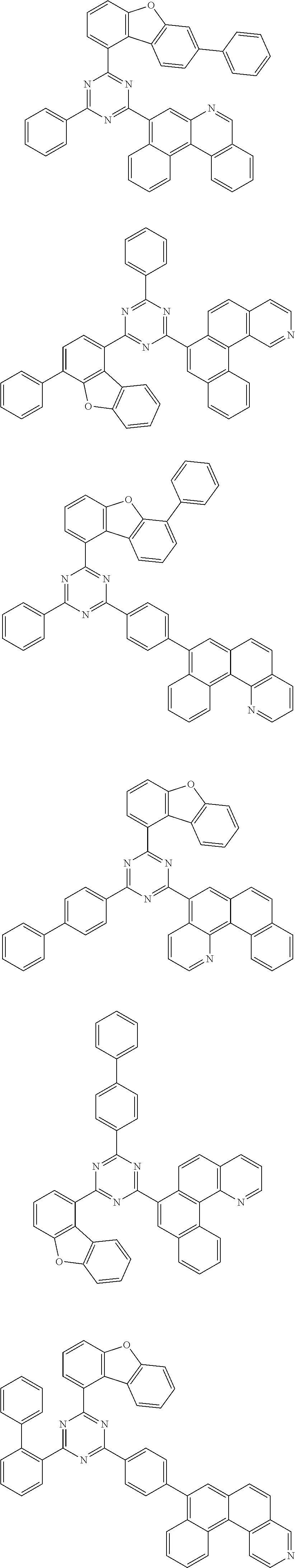

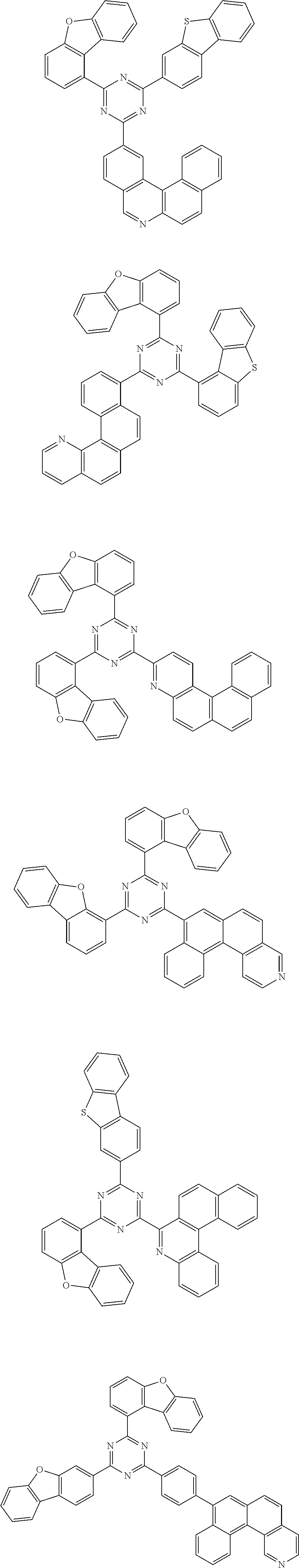

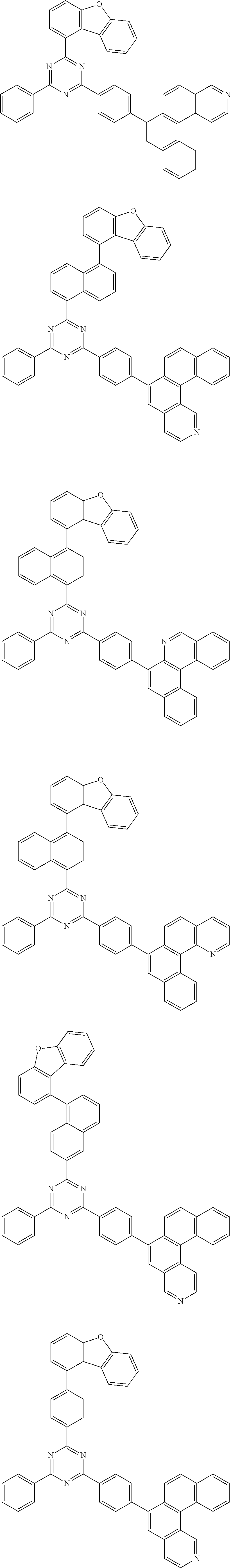

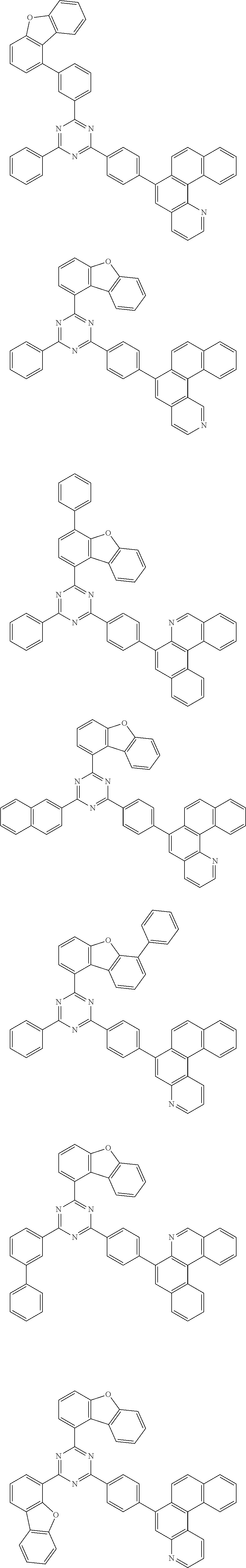

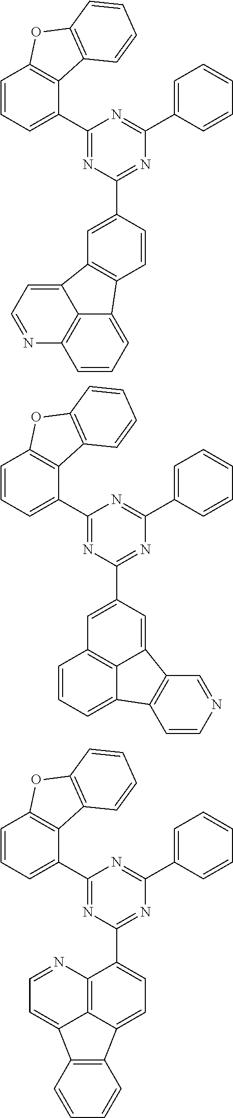

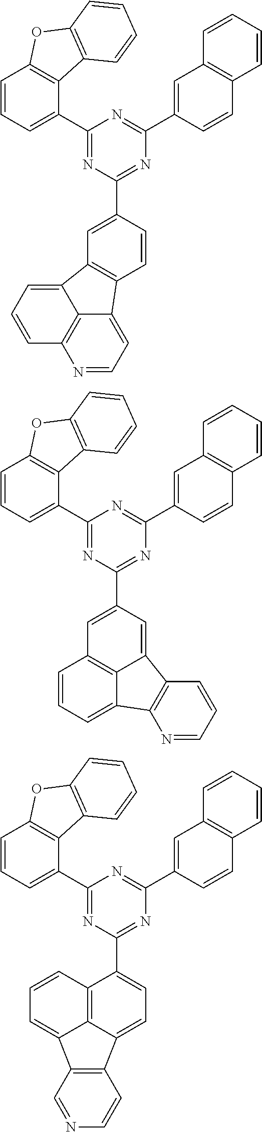

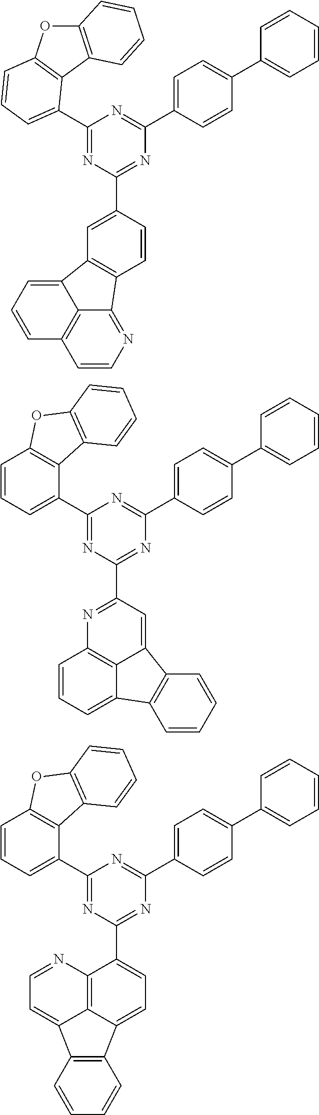

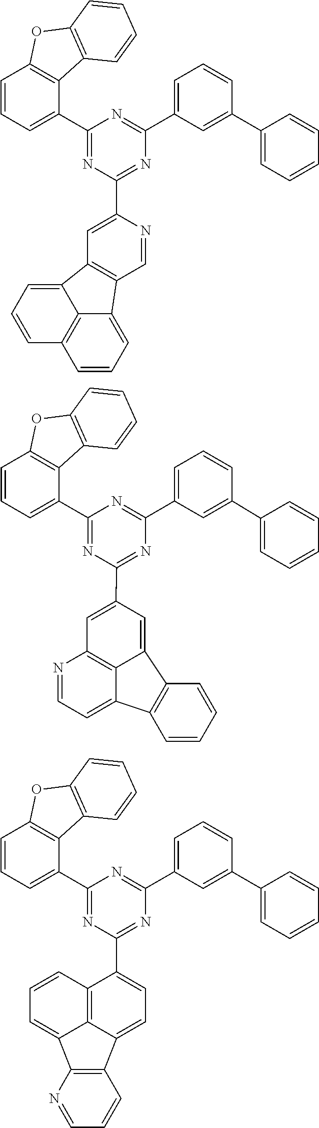

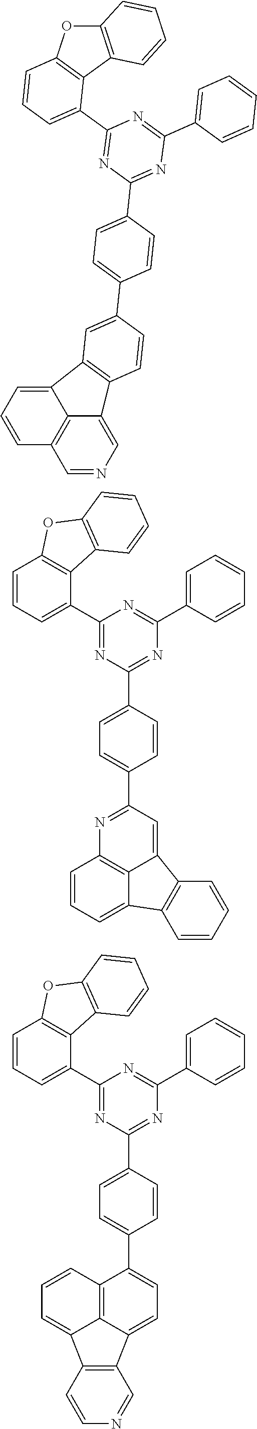

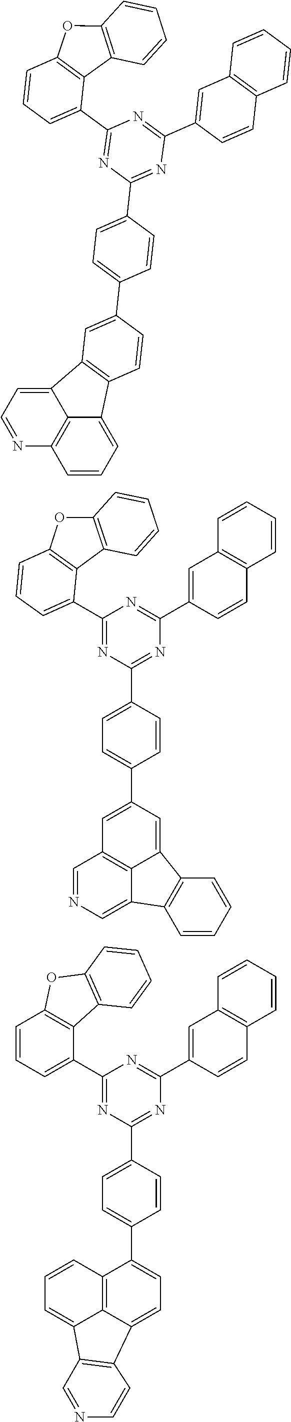

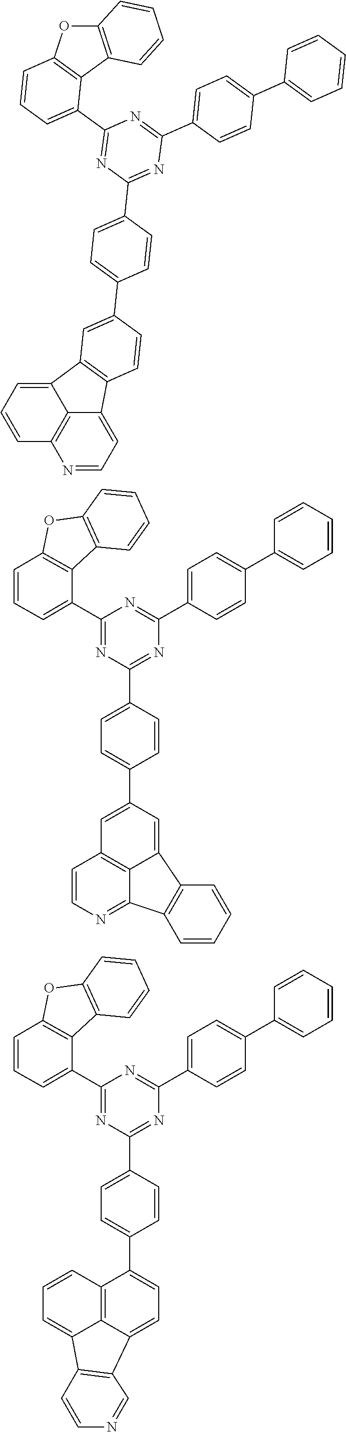

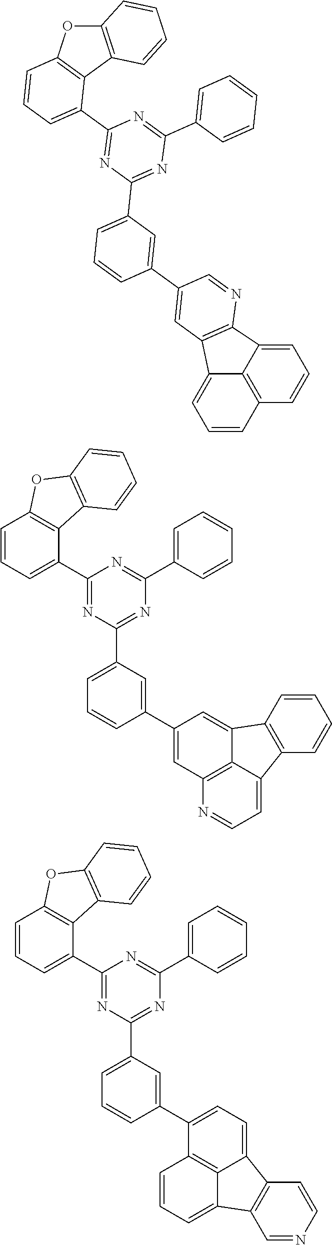 [SMILES: c1ccc(-c2nc(-c3cccc(-c4cnc5c(c4)-c4cccc6cccc-5c46)c3)nc(-c3cccc4oc5ccccc5c34)n2)cc1 ; c1ccc(-c2nc(-c3cccc(-c4cc5c6c(ccnc6c4)-c4ccccc4-5)c3)nc(-c3cccc4oc5ccccc5c34)n2)cc1 ; c1ccc(-c2nc(-c3cccc(-c4ccc5c6c(cccc46)-c4cnccc4-5)c3)nc(-c3cccc4oc5ccccc5c34)n2)cc1]

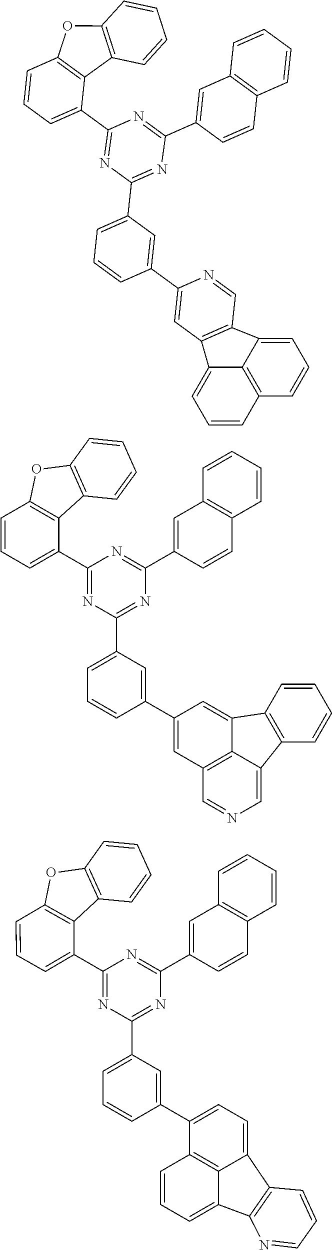

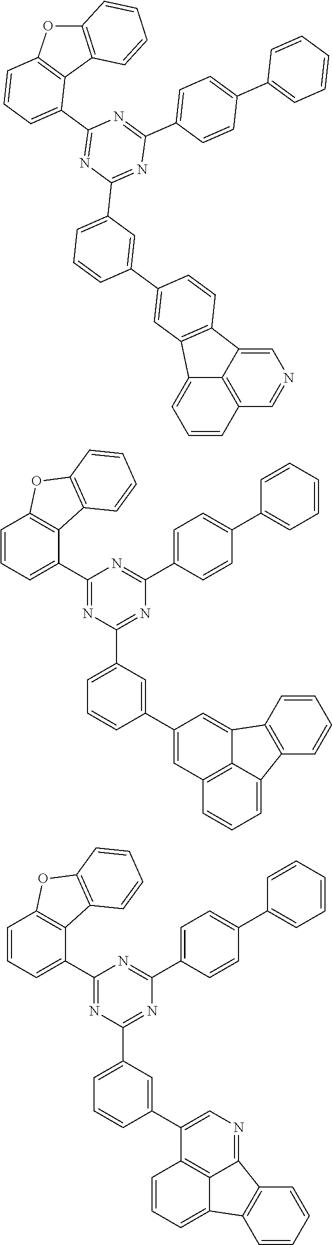 [SMILES: c1ccc(-c2ccc(-c3nc(-c4cccc(-c5ccc6c(c5)-c5cccc7cncc-6c57)c4)nc(-c4cccc5oc6ccccc6c45)n3)cc2)cc1 ; c1ccc(-c2ccc(-c3nc(-c4cccc(-c5cc6c7c(cccc7c5)-c5ccccc5-6)c4)nc(-c4cccc5oc6ccccc6c45)n3)cc2)cc1 ; c1ccc(-c2ccc(-c3nc(-c4cccc(-c5cnc6c7c(cccc57)-c5ccccc5-6)c4)nc(-c4cccc5oc6ccccc6c45)n3)cc2)cc1]

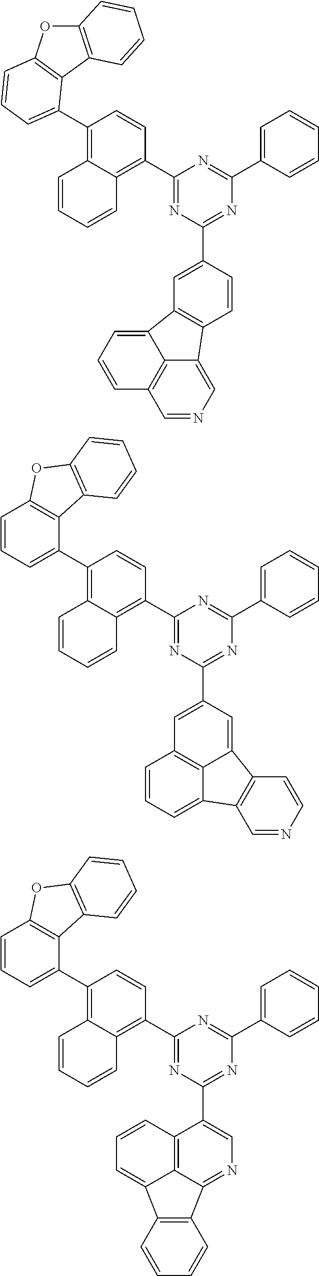 [SMILES: c1ccc(-c2nc(-c3ccc4c(c3)-c3cccc5cncc-4c35)nc(-c3ccc(-c4cccc5oc6ccccc6c45)c4ccccc34)n2)cc1 ; c1ccc(-c2nc(-c3cc4c5c(cccc5c3)-c3cnccc3-4)nc(-c3ccc(-c4cccc5oc6ccccc6c45)c4ccccc34)n2)cc1 ; c1ccc(-c2nc(-c3ccc(-c4cccc5oc6ccccc6c45)c4ccccc34)nc(-c3cnc4c5c(cccc35)-c3ccccc3-4)n2)cc1]

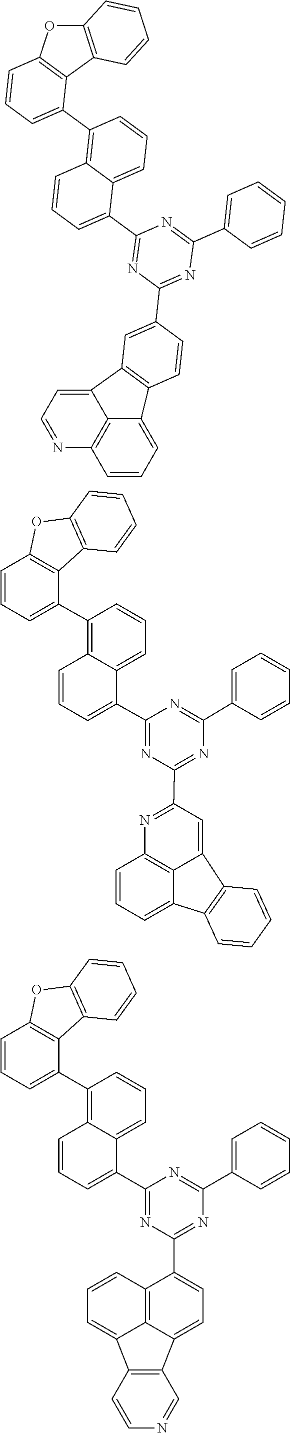

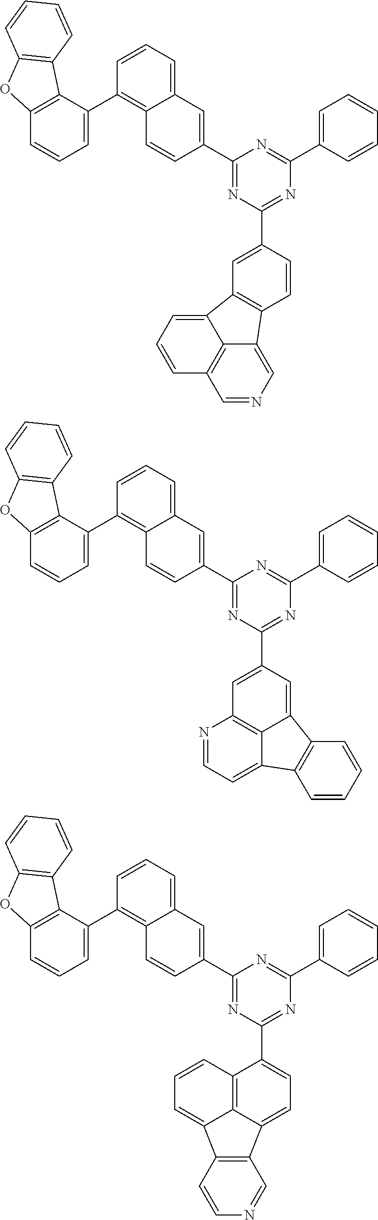 [SMILES: c1ccc(-c2nc(-c3ccc4c(c3)-c3cccc5cncc-4c35)nc(-c3ccc4c(-c5cccc6oc7ccccc7c56)cccc4c3)n2)cc1 ; c1ccc(-c2nc(-c3ccc4c(-c5cccc6oc7ccccc7c56)cccc4c3)nc(-c3cc4c5c(ccnc5c3)-c3ccccc3-4)n2)cc1 ; c1ccc(-c2nc(-c3ccc4c(-c5cccc6oc7ccccc7c56)cccc4c3)nc(-c3ccc4c5c(cccc35)-c3ccncc3-4)n2)cc1]

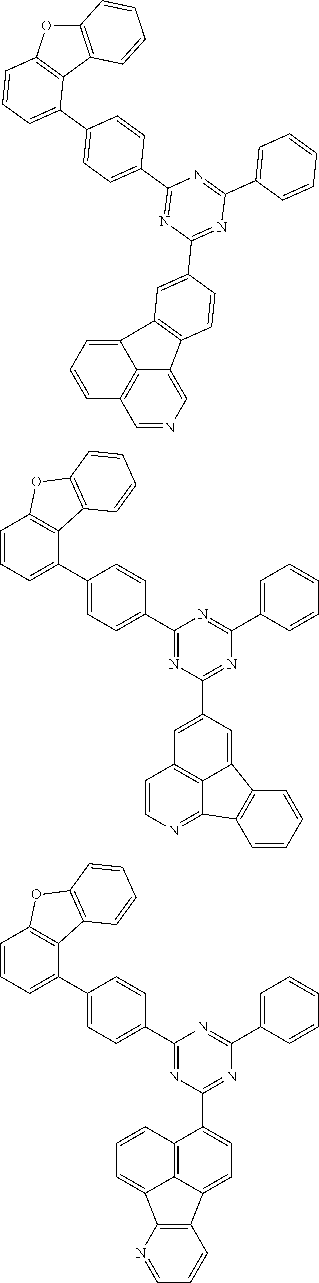

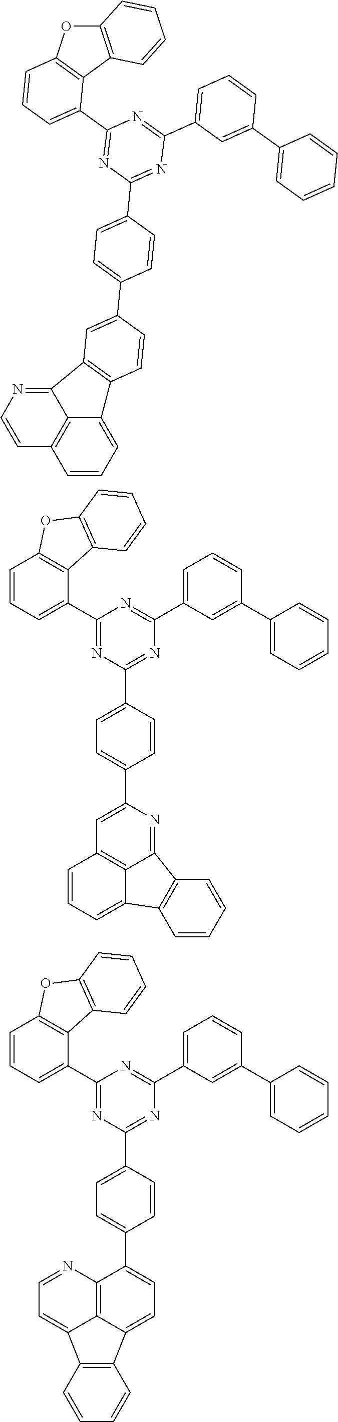

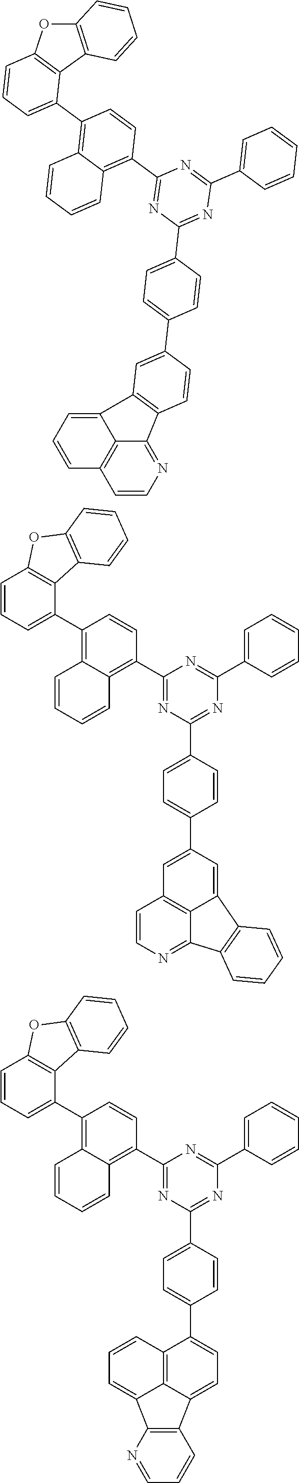

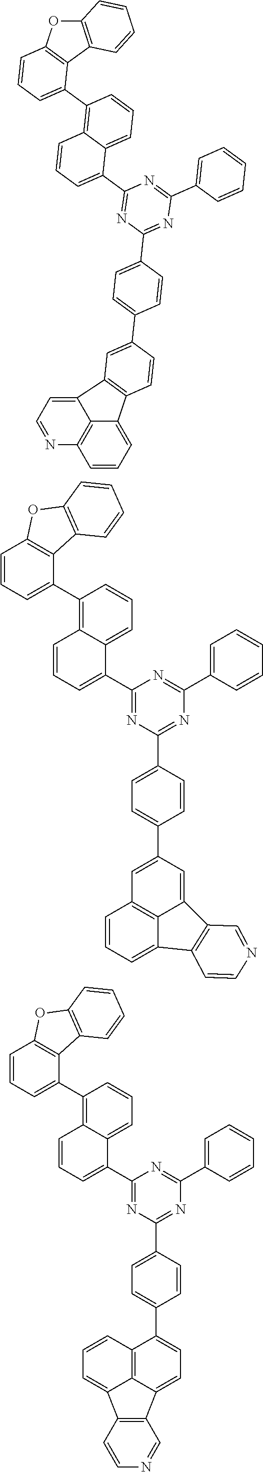

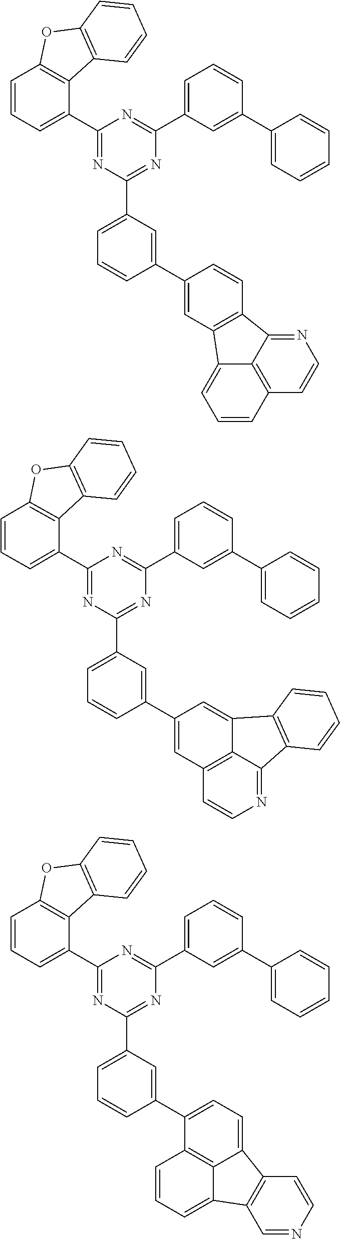 [SMILES: c1ccc(-c2cccc(-c3nc(-c4cccc(-c5ccc6c(c5)-c5cccc7ccnc-6c57)c4)nc(-c4cccc5oc6ccccc6c45)n3)c2)cc1 ; c1ccc(-c2cccc(-c3nc(-c4cccc(-c5cc6c7c(nccc7c5)-c5ccccc5-6)c4)nc(-c4cccc5oc6ccccc6c45)n3)c2)cc1 ; c1ccc(-c2cccc(-c3nc(-c4cccc(-c5ccc6c7c(cccc57)-c5cnccc5-6)c4)nc(-c4cccc5oc6ccccc6c45)n3)c2)cc1]

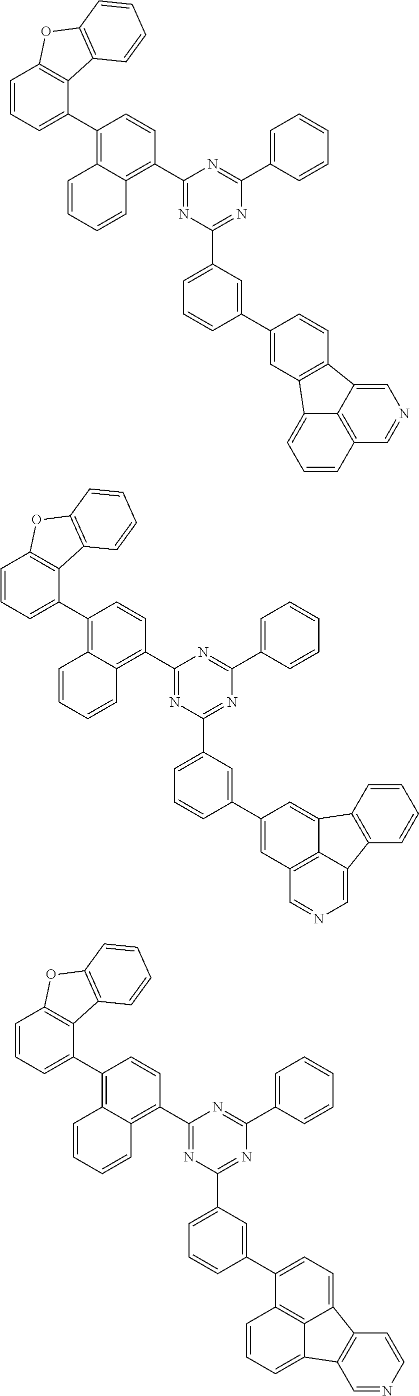 [SMILES: c1ccc(-c2nc(-c3cccc(-c4ccc5c(c4)-c4cccc6cncc-5c46)c3)nc(-c3ccc(-c4cccc5oc6ccccc6c45)c4ccccc34)n2)cc1 ; c1ccc(-c2nc(-c3cccc(-c4cc5c6c(cncc6c4)-c4ccccc4-5)c3)nc(-c3ccc(-c4cccc5oc6ccccc6c45)c4ccccc34)n2)cc1 ; c1ccc(-c2nc(-c3cccc(-c4ccc5c6c(cccc46)-c4cnccc4-5)c3)nc(-c3ccc(-c4cccc5oc6ccccc6c45)c4ccccc34)n2)cc1]

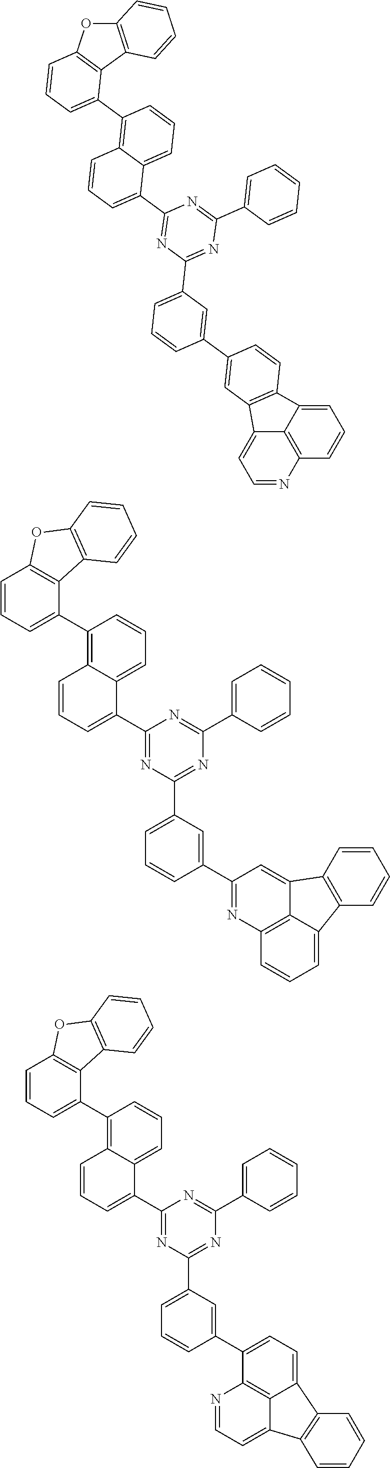 [SMILES: c1ccc(-c2nc(-c3cccc(-c4ccc5c(c4)-c4ccnc6cccc-5c46)c3)nc(-c3cccc4c(-c5cccc6oc7ccccc7c56)cccc34)n2)cc1 ; c1ccc(-c2nc(-c3cccc(-c4cc5c6c(cccc6n4)-c4ccccc4-5)c3)nc(-c3cccc4c(-c5cccc6oc7ccccc7c56)cccc34)n2)cc1 ; c1ccc(-c2nc(-c3cccc(-c4ccc5c6c(ccnc46)-c4ccccc4-5)c3)nc(-c3cccc4c(-c5cccc6oc7ccccc7c56)cccc34)n2)cc1]

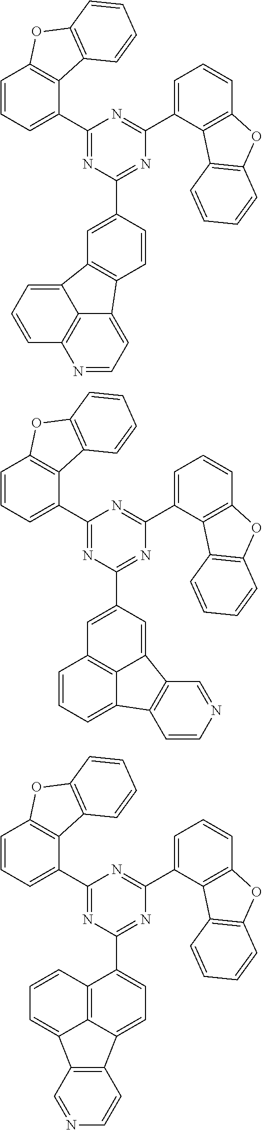

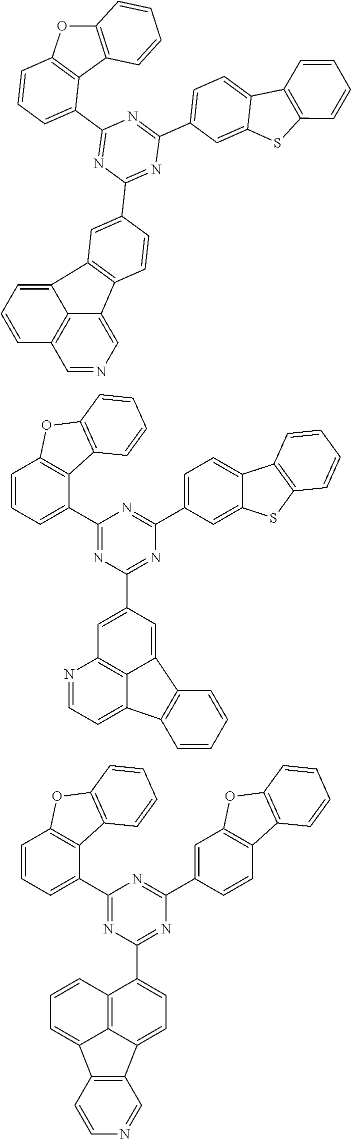

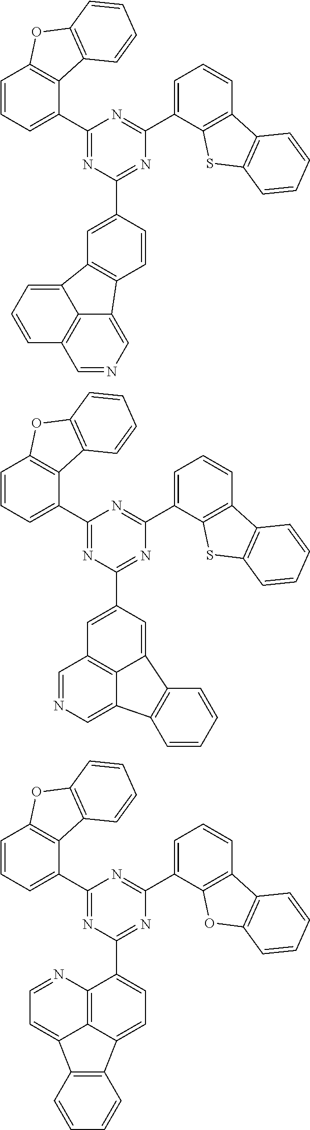

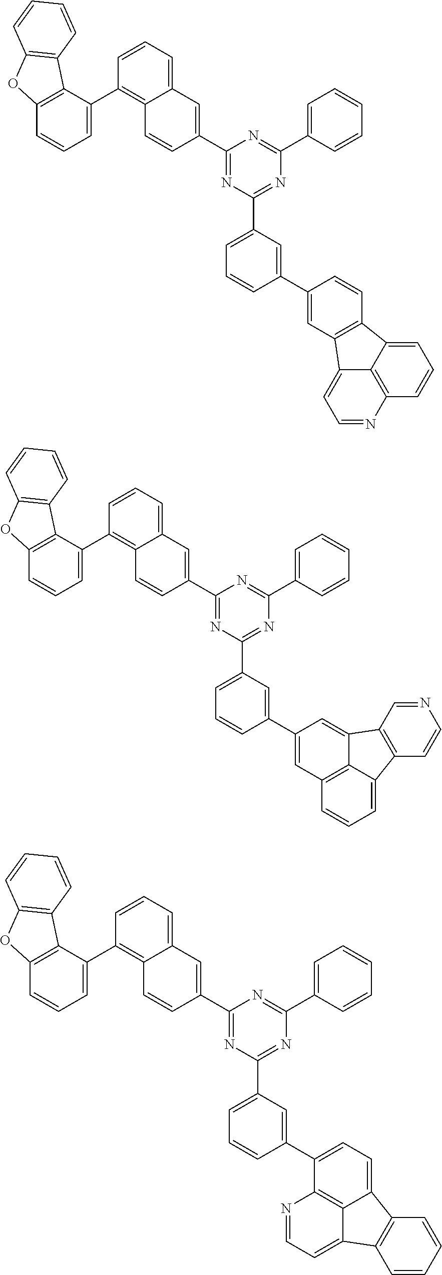 [SMILES: c1ccc(-c2nc(-c3cccc(-c4ccc5c(c4)-c4ccnc6cccc-5c46)c3)nc(-c3ccc4c(-c5cccc6oc7ccccc7c56)cccc4c3)n2)cc1 ; c1ccc(-c2nc(-c3cccc(-c4cc5c6c(cccc6c4)-c4ccncc4-5)c3)nc(-c3ccc4c(-c5cccc6oc7ccccc7c56)cccc4c3)n2)cc1 ; c1ccc(-c2nc(-c3cccc(-c4ccc5c6c(ccnc46)-c4ccccc4-5)c3)nc(-c3ccc4c(-c5cccc6oc7ccccc7c56)cccc4c3)n2)cc1]

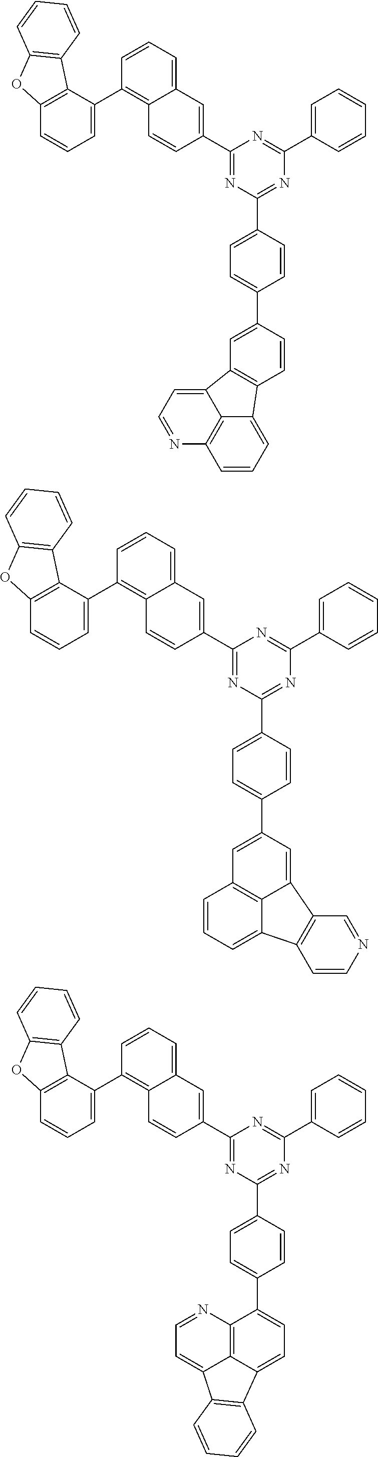 [SMILES: c1ccc(-c2nc(-c3ccc(-c4ccc5c(c4)-c4ccnc6cccc-5c46)cc3)nc(-c3ccc4c(-c5cccc6oc7ccccc7c56)cccc4c3)n2)cc1 ; c1ccc(-c2nc(-c3ccc(-c4cc5c6c(cccc6c4)-c4ccncc4-5)cc3)nc(-c3ccc4c(-c5cccc6oc7ccccc7c56)cccc4c3)n2)cc1 ; c1ccc(-c2nc(-c3ccc(-c4ccc5c6c(ccnc46)-c4ccccc4-5)cc3)nc(-c3ccc4c(-c5cccc6oc7ccccc7c56)cccc4c3)n2)cc1]

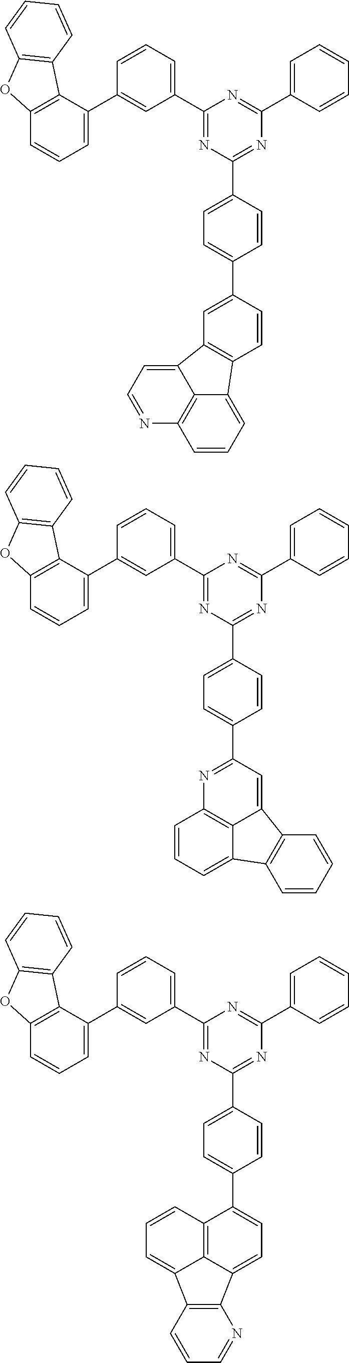 [SMILES: c1ccc(-c2nc(-c3ccc(-c4ccc5c(c4)-c4ccnc6cccc-5c46)cc3)nc(-c3cccc(-c4cccc5oc6ccccc6c45)c3)n2)cc1 ; c1ccc(-c2nc(-c3ccc(-c4cc5c6c(cccc6n4)-c4ccccc4-5)cc3)nc(-c3cccc(-c4cccc5oc6ccccc6c45)c3)n2)cc1 ; c1ccc(-c2nc(-c3ccc(-c4ccc5c6c(cccc46)-c4cccnc4-5)cc3)nc(-c3cccc(-c4cccc5oc6ccccc6c45)c3)n2)cc1]

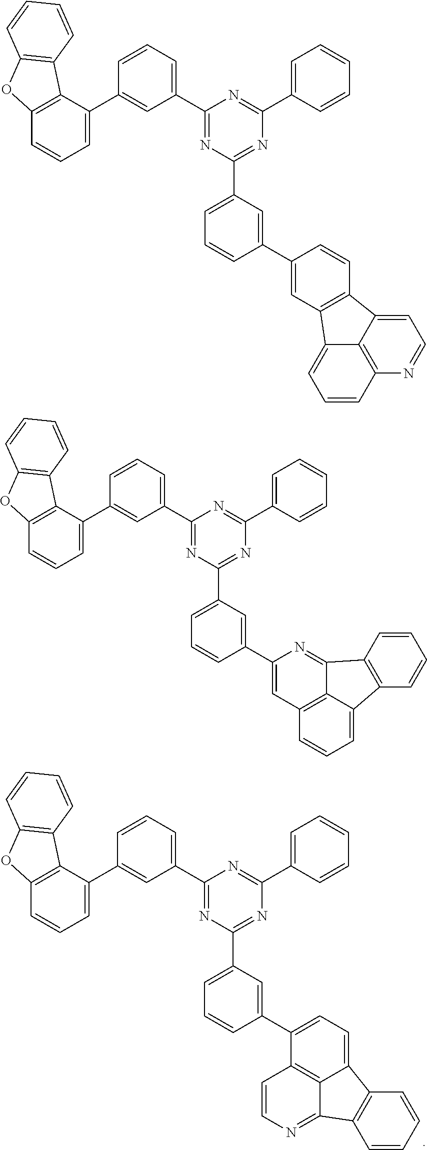 [SMILES: c1ccc(-c2nc(-c3cccc(-c4ccc5c(c4)-c4cccc6nccc-5c46)c3)nc(-c3cccc(-c4cccc5oc6ccccc6c45)c3)n2)cc1 ; c1ccc(-c2nc(-c3cccc(-c4cc5cccc6c5c(n4)-c4ccccc4-6)c3)nc(-c3cccc(-c4cccc5oc6ccccc6c45)c3)n2)cc1 ; c1ccc(-c2nc(-c3cccc(-c4ccc5c6c(nccc46)-c4ccccc4-5)c3)nc(-c3cccc(-c4cccc5oc6ccccc6c45)c3)n2)cc1]

According to the present disclosure, also provided is a method for preparing the compound of Chemical Formula 1 as shown in the following Reaction Scheme 1.

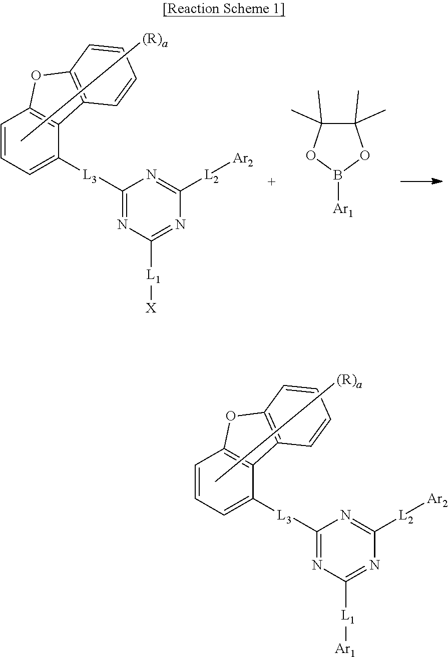

In Reaction Scheme 1, Ar₁, Ar₂, R, L₁, L₂, and L₃ are as defined in Chemical Formula 1. Also, in Reaction Scheme 1, X is halogen, more preferably chloro.

Reaction Scheme 1 is a Suzuki-coupling reaction, which is preferably carried out in the presence of a palladium catalyst and a base, and a reactive group for the Suzuki-coupling reaction can be changed to those known in the art. Such a preparation method can be further embodied in the Synthesis Examples described hereinafter.

### (Organic Light Emitting Device)

Meanwhile, the present disclosure provides an organic light emitting device comprising a compound of Chemical Formula 1. In one example, the present disclosure provides an organic light emitting device comprising: a first electrode; a second electrode that is opposite to the first electrode; and one or more organic material layers that are between the first electrode and the second electrode, wherein one or more layers of the one or more organic material layers includes the compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure comprising a hole injection layer, a hole transport layer, a light emitting layer, an electron transport layer, an electron injection layer and the like as the organic material layer. However, the structure of the organic light emitting device is not limited thereto, and it can include a smaller number of organic layers.

In one embodiment, the organic material layer can include a light emitting layer, wherein the organic material layer including the compound can be a light emitting layer.

In another embodiment, the organic material layer can include a hole injection layer, a hole transport layer, a light emitting layer, and an electron injection and transport layer, wherein the organic material layer including the compound can be a light emitting layer or an electron injection and transport layer.

In another embodiment, the organic material layer can include a hole injection layer, a hole transport layer, an electron blocking layer, a light emitting layer, and an electron injection and transport layer, wherein the organic material layer including the compound can be a light emitting layer or an electron injection and transport layer.

In yet another embodiment, the organic material layer can include a hole injection layer, a hole transport layer, an electron blocking layer, a light emitting layer, an electron inhibition layer, and an electron injection and transport layer, wherein the organic material layer including the compound can be a light emitting layer or an electron injection and transport layer.

The organic material layer of the organic light emitting device of the present disclosure can have a single-layer structure, or it can have a multilayered structure in which two or more organic material layers are stacked. For example, the organic light emitting device of the present disclosure can have a structure further comprising a hole injection layer and a hole transport layer between the first electrode and the light emitting layer, and an electron transport layer and an electron injection layer between the light emitting layer and the second electrode, in addition to the light emitting layer, as the organic material layer. However, the structure of the organic light emitting device is not limited thereto, and it can include a smaller number of organic layers or a larger number of organic layers.

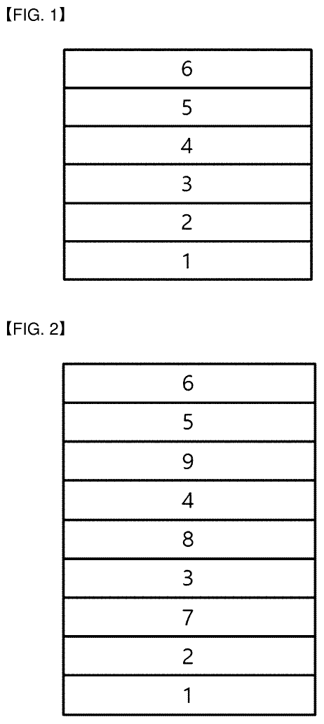

Further, the organic light emitting device according to the present disclosure can be a normal type organic light emitting device in which an anode, one or more organic material layers and a cathode are sequentially stacked on a substrate, wherein the first electrode is an anode, and the second electrode is a cathode. Further, the organic light emitting device according to the present disclosure can be an inverted type organic light emitting device in which a cathode, one or more organic material layers and an anode are sequentially stacked on a substrate, wherein the first electrode is a cathode and the second electrode is an anode. For example, the structure of the organic light emitting device according to one embodiment of the present disclosure is illustrated in FIGS. 1 and 2.

FIG. 1 shows an example of an organic light emitting device comprising a substrate 1, an anode 2, a hole transport layer 3, a light emitting layer 4, an electron injection and transport layer 5 and a cathode 6. In such a structure, the compound of Chemical Formula 1 can be included in the hole transport layer.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6. In such a structure, the compound of Chemical Formula 1 can be included in the hole injection layer, the hole transport layer, or the electron blocking layer.

The organic light emitting device according to the present disclosure can be manufactured by materials and methods known in the art, except that at least one of the organic material layers includes the compound of Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/AI, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. The hole transport material includes an arylamine-based organic material, a conductive polymer, a block copolymer in which a conjugate portion and a non-conjugate portion are present together, and the like, but are not limited thereto.

Further, the electron blocking layer refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include an arylamine-based organic material or the like, but is not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene)(PPV)-based polymer; a spiro compound; polyfluorene, lubrene, and the like, but are not limited thereto.

Further, the light emitting layer can include a host material and a dopant material. The compound of Chemical Formula 1 can be used as such a host material. Further, the host material can further include a fused aromatic ring derivative, a heterocycle-containing compound or the like in addition to the compound of Chemical Formula 1. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer refers to a layer which is formed on the light emitting layer, and preferably, is provided in contact with the light emitting layer, and thus severs to control electron mobility, to prevent excessive movement of holes, and to increase the probability of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq₃; an organic radical compound; a hydroxyflavone-metal complex, a triazine derivative, and the like, but are not limited thereto. Alternatively, it can be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer can also be formed as a separate layer such as an electron injection layer and an electron transport layer. In such a case, the electron transport layer is formed on the light emitting layer or the hole blocking layer, and the above-mentioned electron injection and transport material can be used as the electron transport material included in the electron transport layer. In addition, the electron injection layer is formed on the electron transport layer, and examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

The metal complex compound includes 8-hydroxyquinolinato lithium, bis(8-hydroxyquinolinato)zinc, bis(8-hydroxyquinolinato)copper, bis(8-hydroxy-quinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)-gallium, bis(2-methyl-8-quinolinato)(1-naphtholato)aluminum, bis(2-methyl-8-quinolinato)(2-naphtholato)gallium, and the like, but is not limited thereto.

The organic light emitting device according to the present disclosure can be a bottom emission type device, a top emission type device, or a double side emission type device, and in particular, it can be a bottom emission type light emitting device that requires relatively high luminous efficiency.

In addition, the compound of Chemical Formula 1 can be included in an organic solar cell or an organic transistor in addition to an organic light emitting device.

### Synthesis Example 1

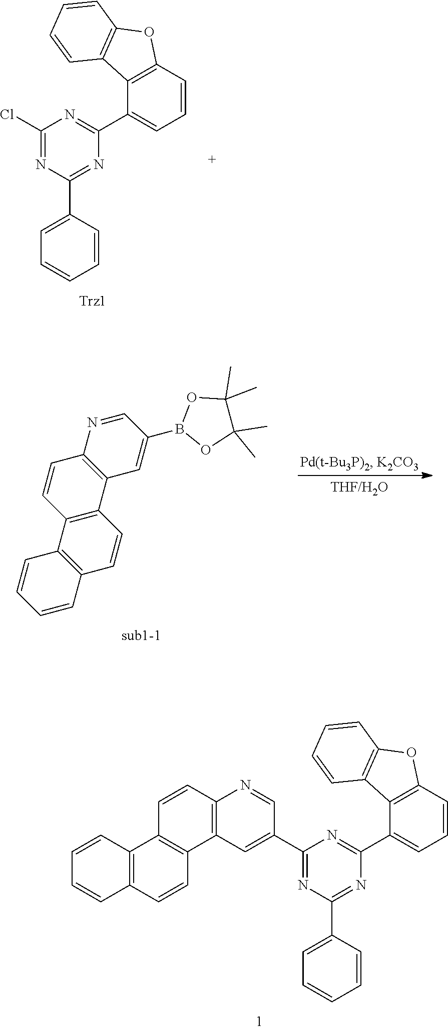 [SMILES: Clc1nc(-c2ccccc2)nc(-c2cccc3oc4ccccc4c23)n1 ; CC1(C)OB(c2cnc3ccc4c5ccccc5ccc4c3c2)OC1(C)C ; c1ccc(-c2nc(-c3cnc4ccc5c6ccccc6ccc5c4c3)nc(-c3cccc4oc5ccccc5c34)n2)cc1]

Trz1 (15 g, 41.9 mmol) and sub1-1 (15.6 g, 44 mmol) were added to 300 ml of THF, and the mixture was stirred and refluxed. Then, potassium carbonate (17.4 g, 125.8 mmol) was dissolved in 100 ml of water and added thereto, and the mixture was sufficiently stirred and then bis(tri-tert-butylphosphine)palladium(0) (0.2 g, 0.4 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 18.5 g of Compound 1. (Yield: 80%, MS: [M+H]⁺=551)

### Synthesis Example 2

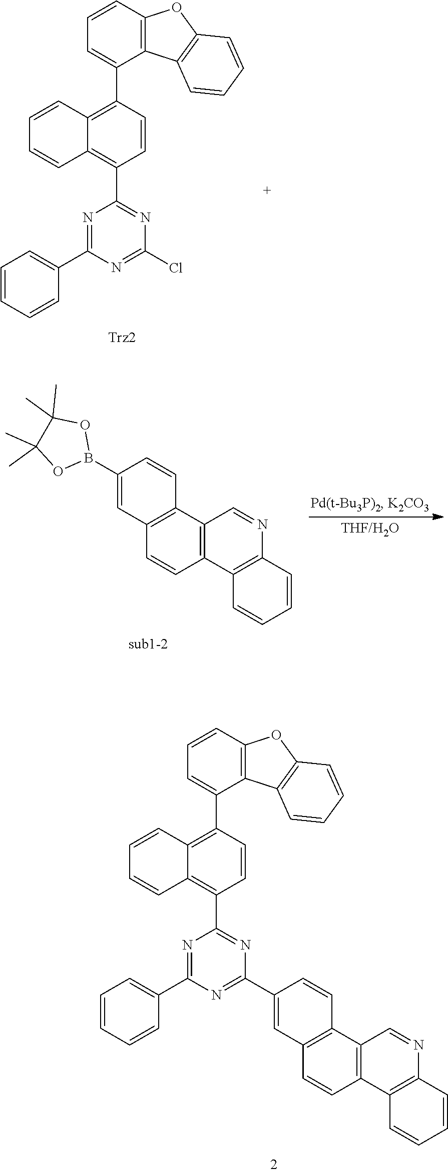 [SMILES: Clc1nc(-c2ccccc2)nc(-c2ccc(-c3cccc4oc5ccccc5c34)c3ccccc23)n1 ; CC1(C)OB(c2ccc3c(ccc4c5ccccc5ncc34)c2)OC1(C)C ; c1ccc(-c2nc(-c3ccc4c(ccc5c6ccccc6ncc45)c3)nc(-c3ccc(-c4cccc5oc6ccccc6c45)c4ccccc34)n2)cc1]

Trz2 (15 g, 31 mmol) and sub1-2 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.6 g of Compound 2. (Yield: 79%, MS:[M+H]⁺=677)

### Synthesis Example 3

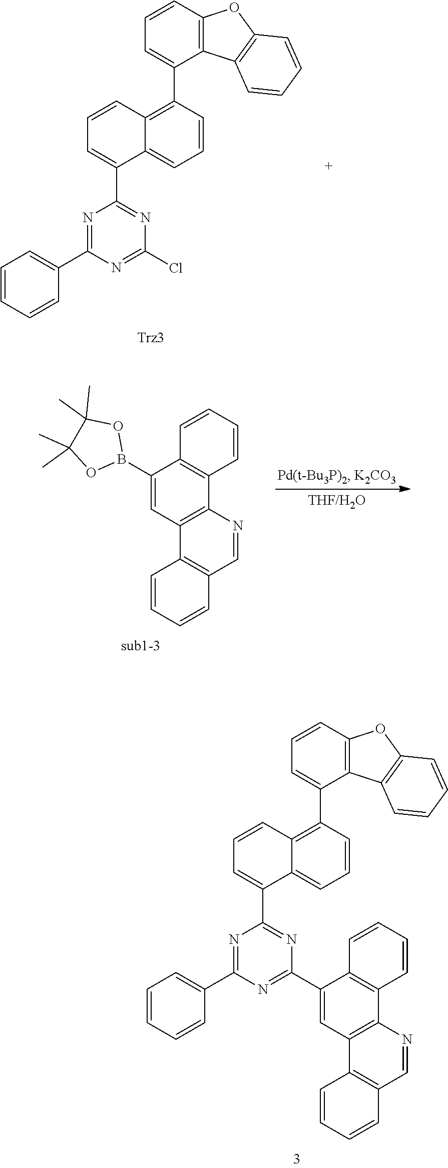 [SMILES: Clc1nc(-c2ccccc2)nc(-c2cccc3c(-c4cccc5oc6ccccc6c45)cccc23)n1 ; CC1(C)OB(c2cc3c4ccccc4cnc3c3ccccc23)OC1(C)C ; c1ccc(-c2nc(-c3cccc4c(-c5cccc6oc7ccccc7c56)cccc34)nc(-c3cc4c5ccccc5cnc4c4ccccc34)n2)cc1]

Trz3 (15 g, 31 mmol) and sub1-3 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.2 g of Compound 3. (Yield: 63%, MS:[M+H]⁺=677)

### Synthesis Example 4

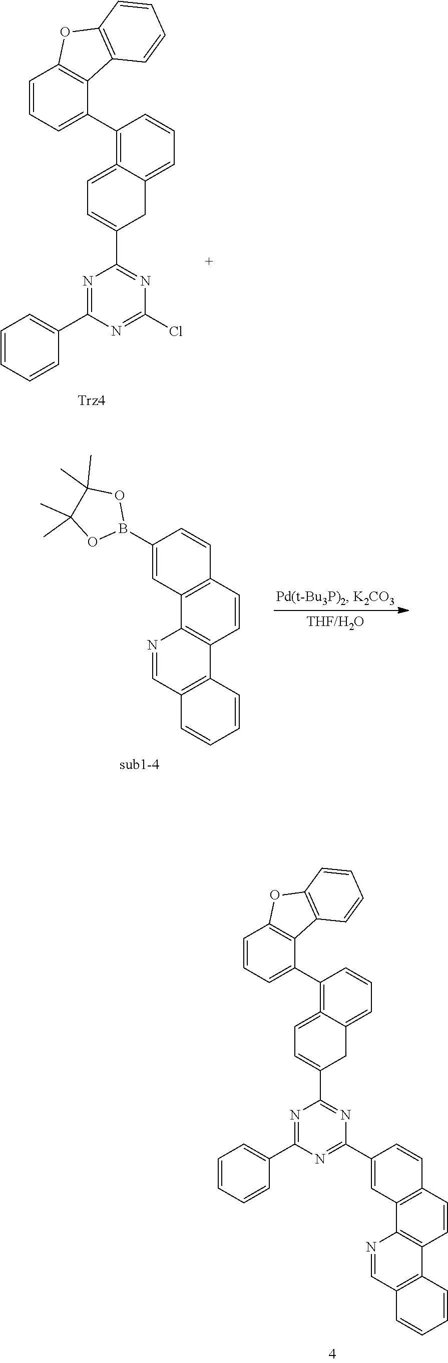

Trz4 (15 g, 31 mmol) and sub1-4 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.5 g of Compound 4. (Yield: 69%, MS:[M+H]⁺=677)

### Synthesis Example 5

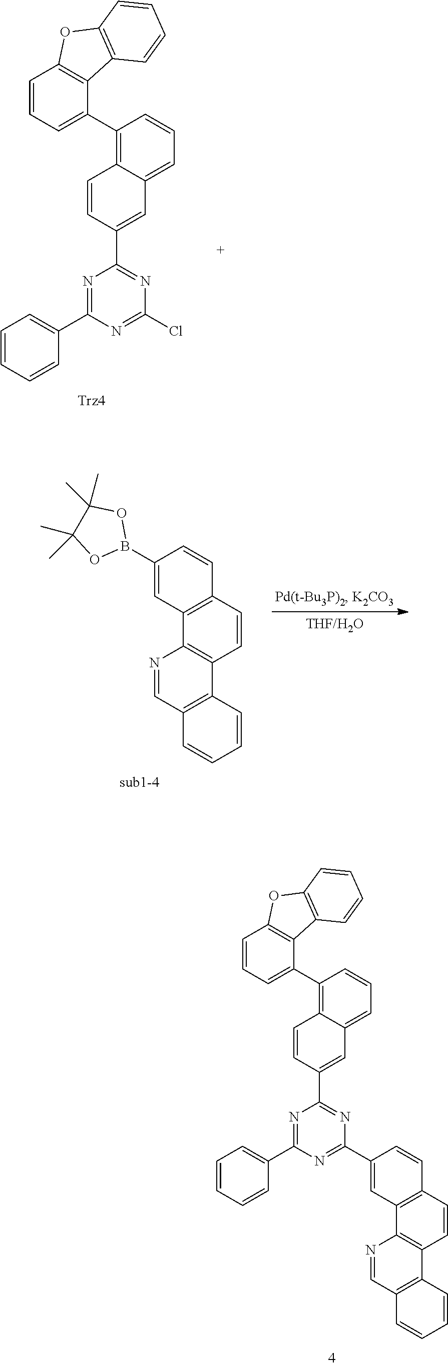 [SMILES: Clc1nc(-c2ccccc2)nc(-c2ccc3c(-c4cccc5oc6ccccc6c45)cccc3c2)n1 ; CC1(C)OB(c2ccc3ccc4c5ccccc5cnc4c3c2)OC1(C)C ; c1ccc(-c2nc(-c3ccc4c(-c5cccc6oc7ccccc7c56)cccc4c3)nc(-c3ccc4ccc5c6ccccc6cnc5c4c3)n2)cc1]

Trz5 (15 g, 26.8 mmol) and sub1-5 (10 g, 28.1 mmol) were added to 300 ml of THF, and the mixture was stirred and refluxed. Then, potassium carbonate (11.1 g, 80.3 mmol) was dissolved in 100 ml of water and added thereto, and the mixture was sufficiently stirred and then bis(tri-tert-butylphosphine)palladium(0) (0.1 g, 0.3 mmol) was added. After the reaction for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.5 g of Compound 5. (Yield: 62%, MS:[M+H]⁺=753)

### Synthesis Example 6

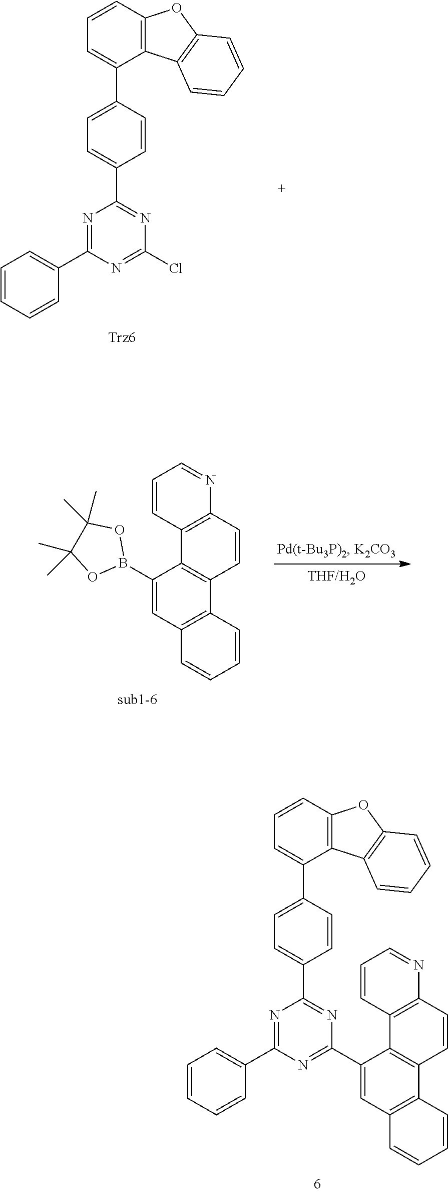 [SMILES: Clc1nc(-c2ccccc2)nc(-c2ccc(-c3cccc4oc5ccccc5c34)cc2)n1 ; CC1(C)OB(c2cc3ccccc3c3ccc4ncccc4c23)OC1(C)C ; c1ccc(-c2nc(-c3ccc(-c4cccc5oc6ccccc6c45)cc3)nc(-c3cc4ccccc4c4ccc5ncccc5c34)n2)cc1]

Trz6 (15 g, 34.6 mmol) and sub1-6 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.9 g of Compound 6. (Yield: 69%, MS:[M+H]⁺=627)

### Synthesis Example 7

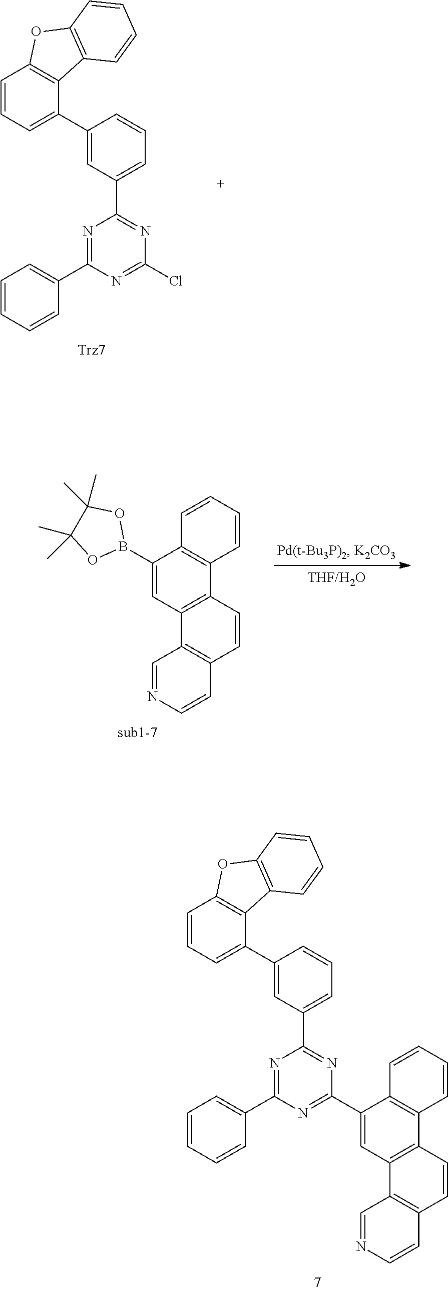 [SMILES: Clc1nc(-c2ccccc2)nc(-c2cccc(-c3cccc4oc5ccccc5c34)c2)n1 ; CC1(C)OB(c2cc3c4cnccc4ccc3c3ccccc23)OC1(C)C ; c1ccc(-c2nc(-c3cccc(-c4cccc5oc6ccccc6c45)c3)nc(-c3cc4c5cnccc5ccc4c4ccccc34)n2)cc1]

Trz7 (15 g, 34.6 mmol) and sub1-7 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.5 g of Compound 7. (Yield: 76%, MS:[M+H]⁺=627)

### Synthesis Example 8

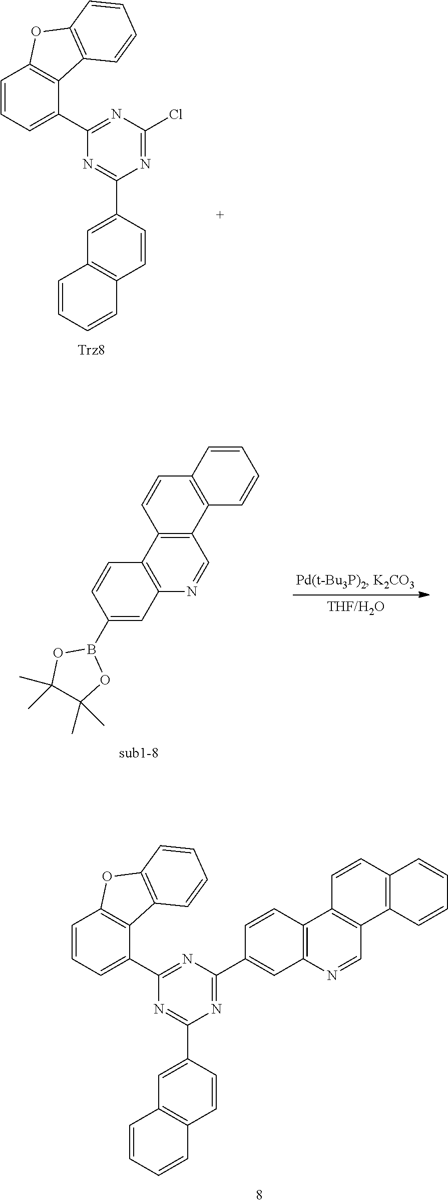 [SMILES: Clc1nc(-c2ccc3ccccc3c2)nc(-c2cccc3oc4ccccc4c23)n1 ; CC1(C)OB(c2ccc3c(c2)ncc2c4ccccc4ccc32)OC1(C)C ; c1ccc2cc(-c3nc(-c4ccc5c(c4)ncc4c6ccccc6ccc54)nc(-c4cccc5oc6ccccc6c45)n3)ccc2c1]

Trz8 (15 g, 36.8 mmol) and sub1-8 (13.7 g, 38.6 mmol) were added to 300 ml of THF, and the mixture was stirred and refluxed. Then, potassium carbonate (15.2 g, 110.3 mmol) was dissolved in 100 ml of water and added thereto, and the mixture was sufficiently stirred and then bis(tri-tert-butylphosphine)palladium(0) (0.2 g, 0.4 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.6 g of Compound 8. (Yield: 66%, MS:[M+H]⁺=601)

### Synthesis Example 9

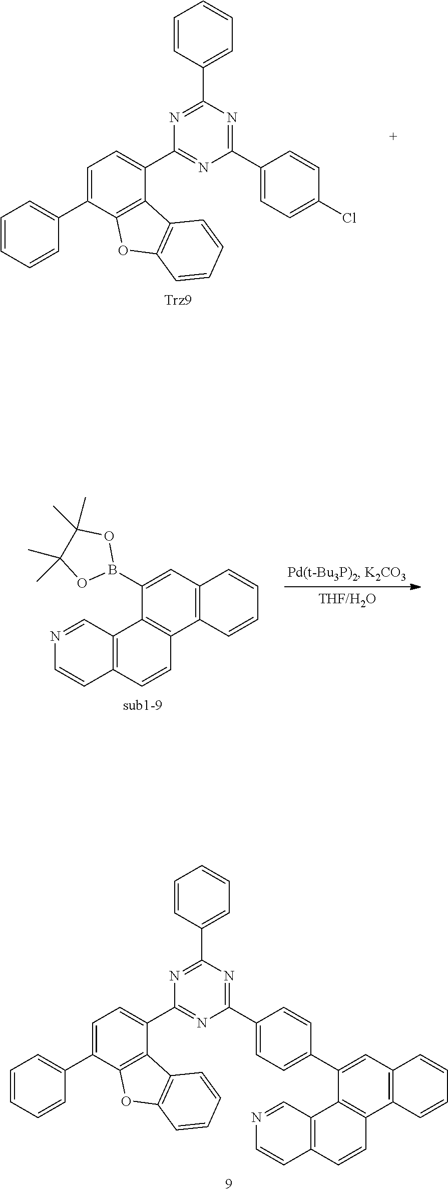 [SMILES: Clc1ccc(-c2nc(-c3ccccc3)nc(-c3ccc(-c4ccccc4)c4oc5ccccc5c34)n2)cc1 ; CC1(C)OB(c2cc3ccccc3c3ccc4ccncc4c23)OC1(C)C ; c1ccc(-c2nc(-c3ccc(-c4cc5ccccc5c5ccc6ccncc6c45)cc3)nc(-c3ccc(-c4ccccc4)c4oc5ccccc5c34)n2)cc1]

Trz9 (15 g, 29.4 mmol) and sub1-9 (11 g, 30.9 mmol) were added to 300 ml of THF, and the mixture was stirred and refluxed. Then, potassium carbonate (12.2 g, 88.2 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.5 g of Compound 9. (Yield: 75%, MS:[M+H]⁺=703)

### Synthesis Example 10

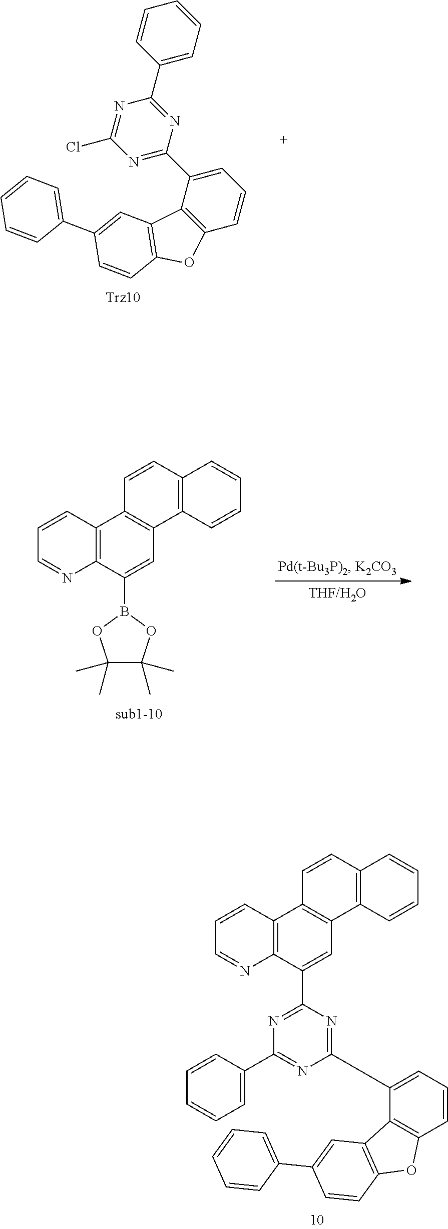 [SMILES: Clc1nc(-c2ccccc2)nc(-c2cccc3oc4ccc(-c5ccccc5)cc4c23)n1 ; CC1(C)OB(c2cc3c4ccccc4ccc3c3cccnc23)OC1(C)C ; c1ccc(-c2ccc3oc4cccc(-c5nc(-c6ccccc6)nc(-c6cc7c8ccccc8ccc7c7cccnc67)n5)c4c3c2)cc1]

Trz10 (15 g, 34.6 mmol) and sub1-10 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.2 g of Compound 10. (Yield: 70%, MS:[M+H]⁺=627)

### Synthesis Example 11

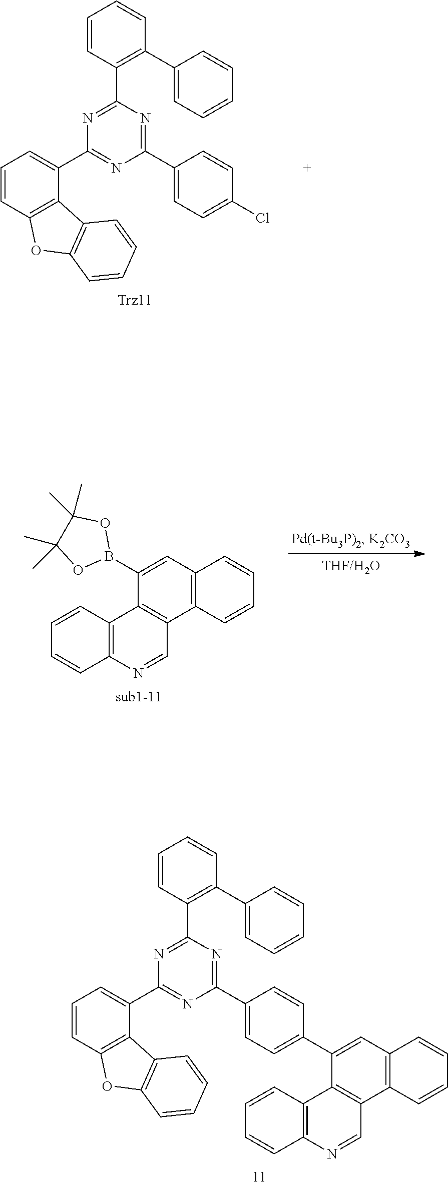 [SMILES: Clc1ccc(-c2nc(-c3ccccc3-c3ccccc3)nc(-c3cccc4oc5ccccc5c34)n2)cc1 ; CC1(C)OB(c2cc3ccccc3c3cnc4ccccc4c23)OC1(C)C ; c1ccc(-c2ccccc2-c2nc(-c3ccc(-c4cc5ccccc5c5cnc6ccccc6c45)cc3)nc(-c3cccc4oc5ccccc5c34)n2)cc1]

Trz11 (15 g, 29.4 mmol) and sub1-11 (11 g, 30.9 mmol) were added to 300 ml of THF, and the mixture was stirred and refluxed. Then, potassium carbonate (12.2 g, 88.2 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.4 g of Compound 11. (Yield: 60%, MS:[M+H]⁺=703)

### Synthesis Example 12

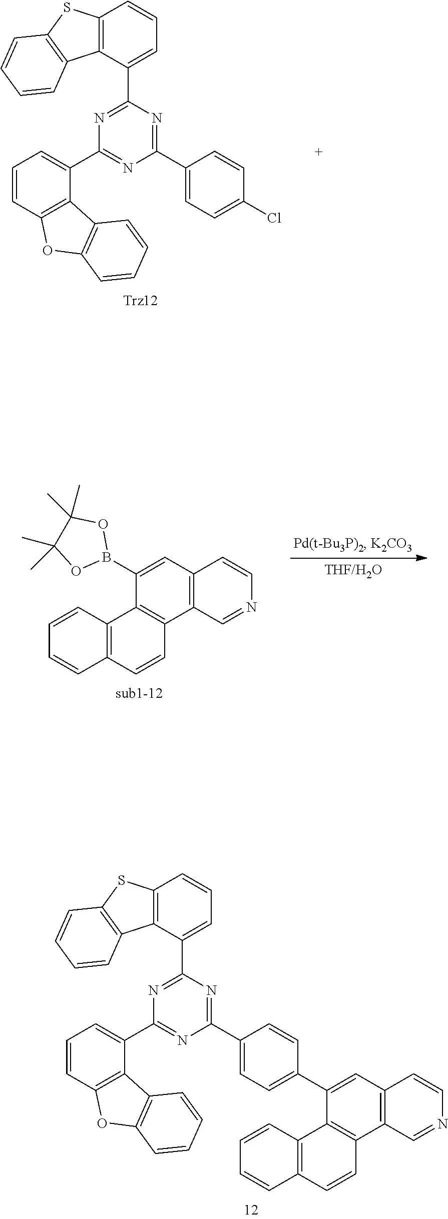 [SMILES: Clc1ccc(-c2nc(-c3cccc4oc5ccccc5c34)nc(-c3cccc4sc5ccccc5c34)n2)cc1 ; CC1(C)OB(c2cc3ccncc3c3ccc4ccccc4c23)OC1(C)C ; c1ccc2c(c1)ccc1c3cnccc3cc(-c3ccc(-c4nc(-c5cccc6oc7ccccc7c56)nc(-c5cccc6sc7ccccc7c56)n4)cc3)c21]

Trz12 (15 g, 27.8 mmol) and sub1-12 (10.4 g, 29.2 mmol) were added to 300 ml of THF, and the mixture was stirred and refluxed. Then, potassium carbonate (11.5 g, 83.3 mmol) was dissolved in 100 ml of water and added thereto, and the mixture was sufficiently stirred and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13 g of Compound 12. (Yield: 64%, MS:[M+H]⁺=733)

### Synthesis Example 13

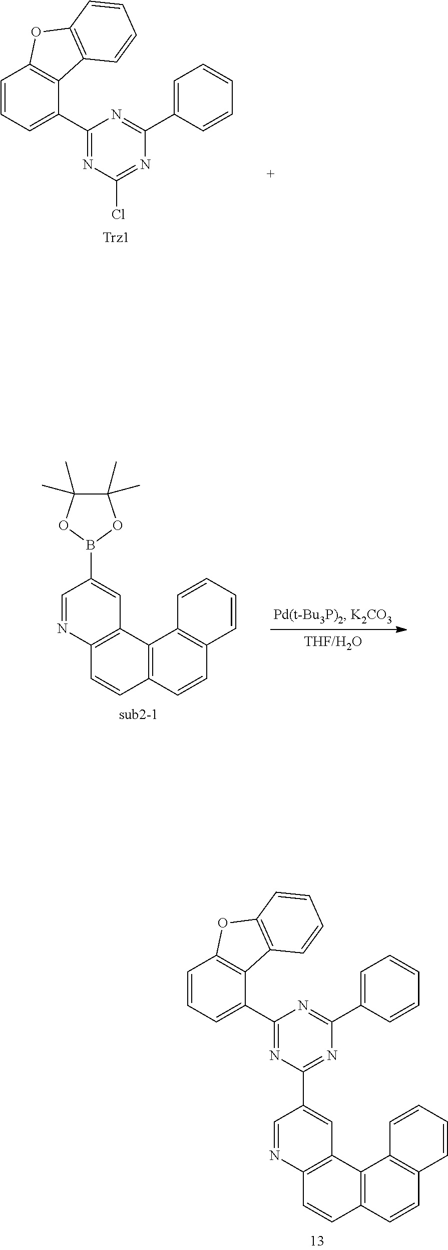 [SMILES: Clc1nc(-c2ccccc2)nc(-c2cccc3oc4ccccc4c23)n1 ; CC1(C)OB(c2cnc3ccc4ccc5ccccc5c4c3c2)OC1(C)C ; c1ccc(-c2nc(-c3cnc4ccc5ccc6ccccc6c5c4c3)nc(-c3cccc4oc5ccccc5c34)n2)cc1]

Trz1 (15 g, 41.9 mmol) and sub2-1 (15.6 g, 44 mmol) were added to 300 ml of THF, and the mixture was stirred and refluxed. Then, potassium carbonate (17.4 g, 125.8 mmol) was dissolved in 100 ml of water and added thereto, and the mixture was sufficiently stirred and then bis(tri-tert-butylphosphine)palladium(0) (0.2 g, 0.4 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.8 g of Compound 13. (Yield: 73%, MS:[M+H]⁺=551)

### Synthesis Example 14

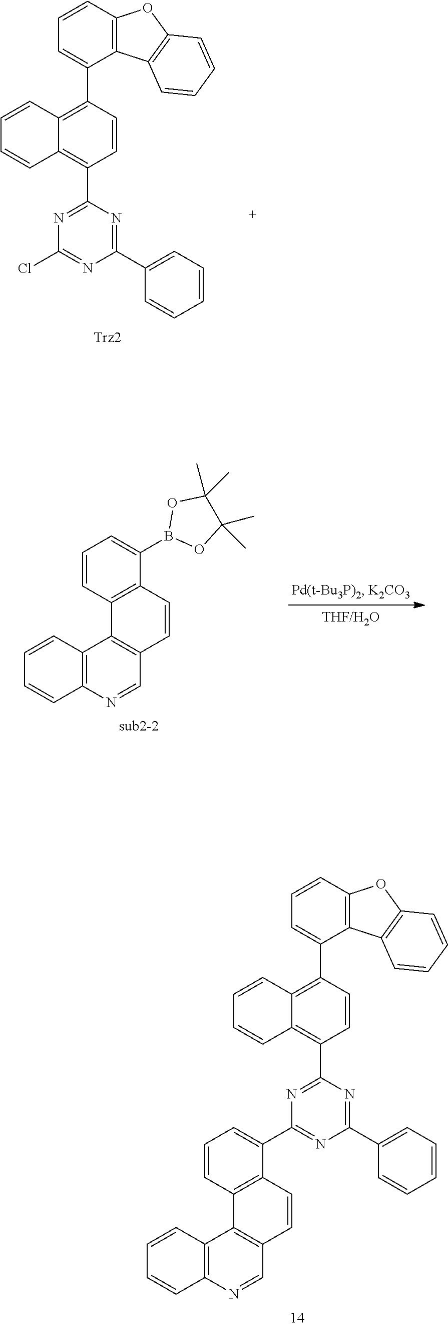 [SMILES: Clc1nc(-c2ccccc2)nc(-c2ccc(-c3cccc4oc5ccccc5c34)c3ccccc23)n1 ; CC1(C)OB(c2cccc3c2ccc2cnc4ccccc4c23)OC1(C)C ; c1ccc(-c2nc(-c3ccc(-c4cccc5oc6ccccc6c45)c4ccccc34)nc(-c3cccc4c3ccc3cnc5ccccc5c34)n2)cc1]

Trz2 (15 g, 31 mmol) and sub2-2 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound 14. (Yield: 66%, MS:[M+H]⁺=677)

### Synthesis Example 15

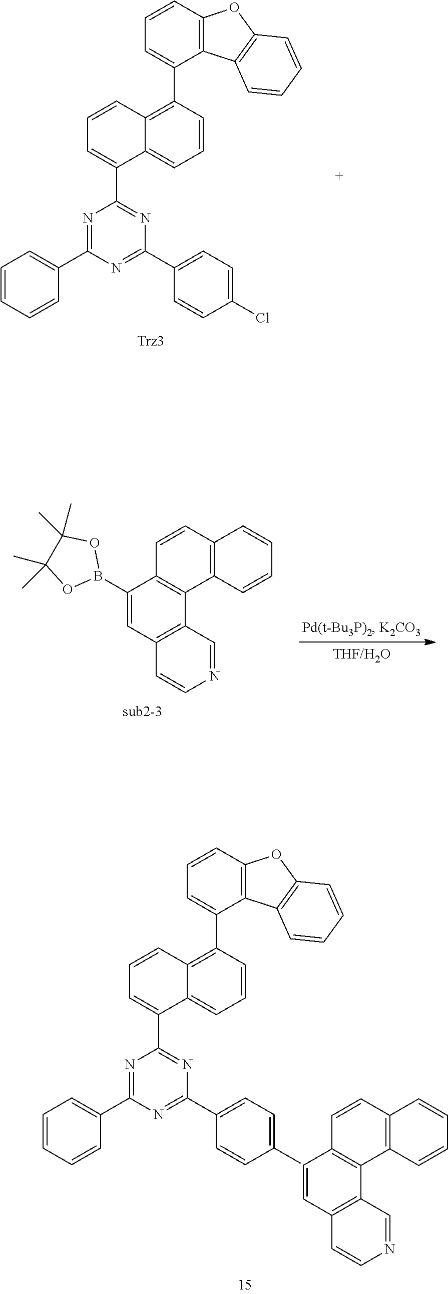

Trz3 (15 g, 31 mmol) and sub2-3 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.8 g of Compound 15. (Yield: 80%, MS:[M+H]⁺=677)

### Synthesis Example 16

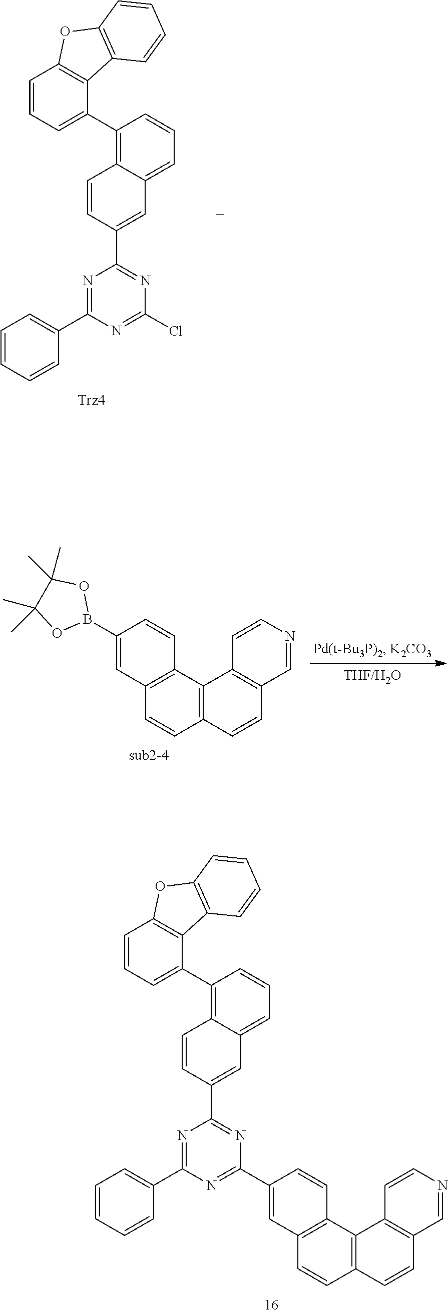 [SMILES: Clc1nc(-c2ccccc2)nc(-c2ccc3c(-c4cccc5oc6ccccc6c45)cccc3c2)n1 ; CC1(C)OB(c2ccc3c(ccc4ccc5cnccc5c43)c2)OC1(C)C ; c1ccc(-c2nc(-c3ccc4c(-c5cccc6oc7ccccc7c56)cccc4c3)nc(-c3ccc4c(ccc5ccc6cnccc6c54)c3)n2)cc1]

Trz4 (15 g, 31 mmol) and sub2-4 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14 g of Compound 16. (Yield: 67%, MS:[M+H]⁺=677)

### Synthesis Example 17

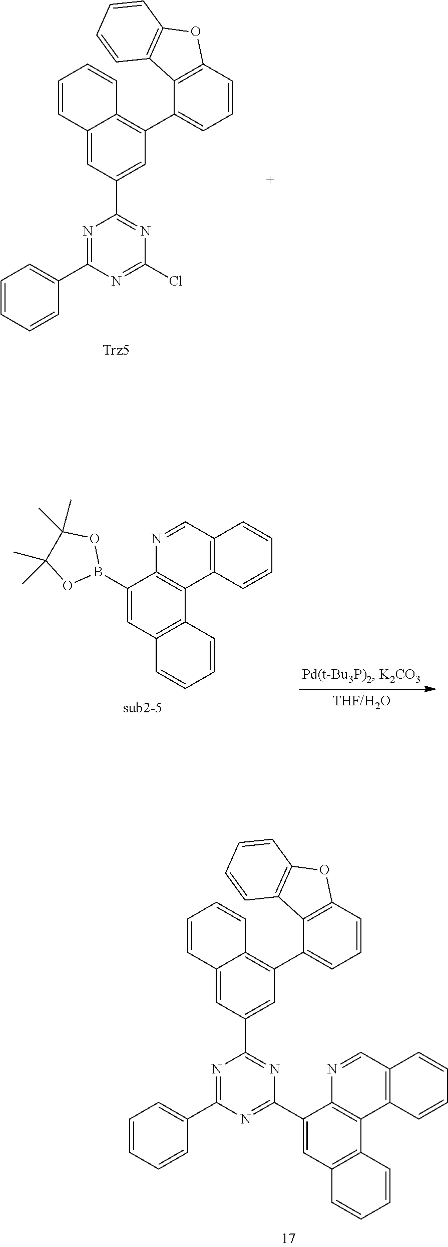 [SMILES: Clc1nc(-c2ccccc2)nc(-c2cc(-c3cccc4oc5ccccc5c34)c3ccccc3c2)n1 ; CC1(C)OB(c2cc3ccccc3c3c2ncc2ccccc23)OC1(C)C ; c1ccc(-c2nc(-c3cc(-c4cccc5oc6ccccc6c45)c4ccccc4c3)nc(-c3cc4ccccc4c4c3ncc3ccccc34)n2)cc1]

Trz5 (15 g, 26.8 mmol) and sub2-5 (10 g, 28.1 mmol) were added to 300 ml of THF, and the mixture was stirred and refluxed. Then, potassium carbonate (11.1 g, 80.3 mmol) was dissolved in 100 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.9 g of Compound 17. (Yield: 79%, MS:[M+H]⁺=753)

### Synthesis Example 18

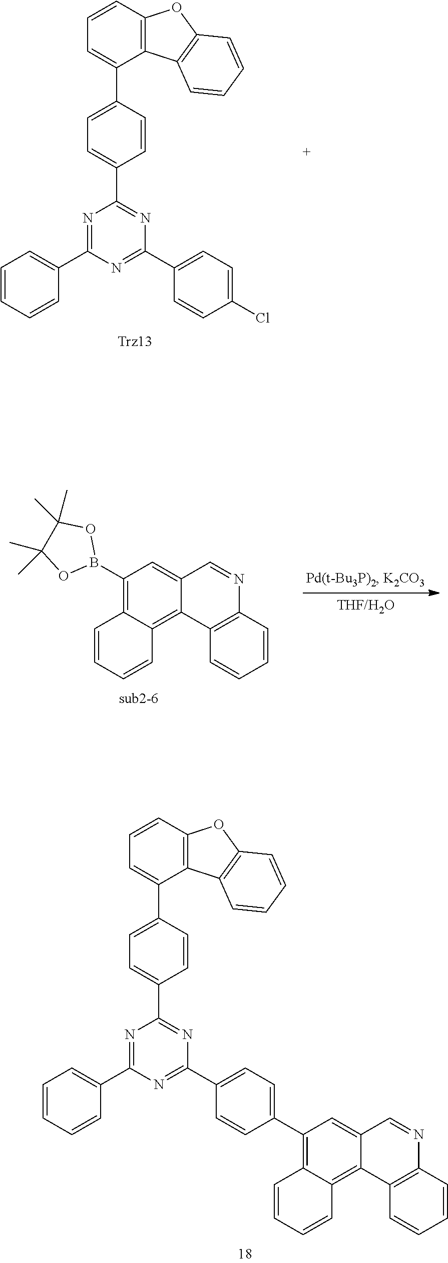 [SMILES: Clc1ccc(-c2nc(-c3ccccc3)nc(-c3ccc(-c4cccc5oc6ccccc6c45)cc3)n2)cc1 ; CC1(C)OB(c2cc3cnc4ccccc4c3c3ccccc23)OC1(C)C ; c1ccc(-c2nc(-c3ccc(-c4cc5cnc6ccccc6c5c5ccccc45)cc3)nc(-c3ccc(-c4cccc5oc6ccccc6c45)cc3)n2)cc1]

Trz13 (15 g, 29.4 mmol) and sub2-6 (11 g, 30.9 mmol) were added to 300 ml of THF, and the mixture was stirred and refluxed. Then, potassium carbonate (12.2 g, 88.2 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.9 g of Compound 18. (Yield: 77%, MS:[M+H]⁺=703)

### Synthesis Example 19

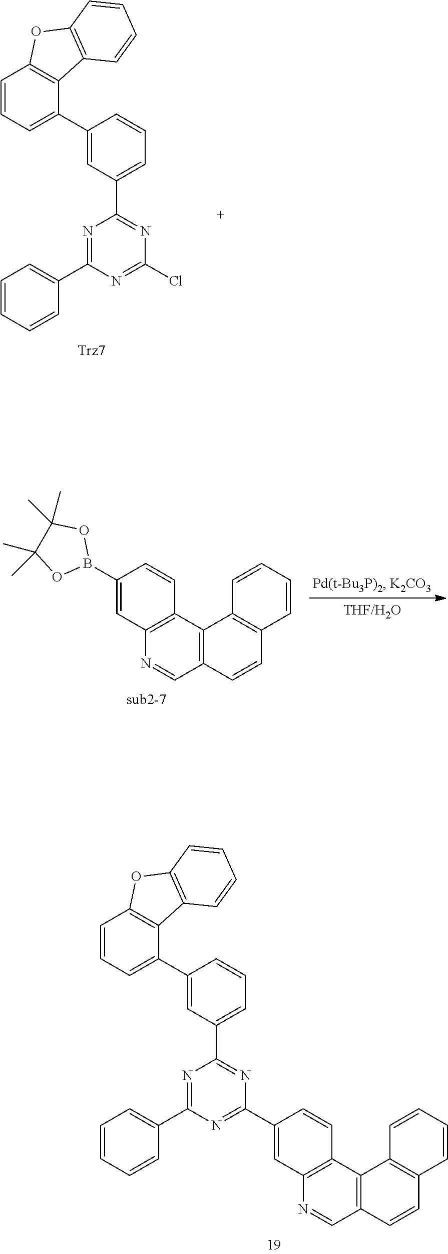 [SMILES: Clc1nc(-c2ccccc2)nc(-c2cccc(-c3cccc4oc5ccccc5c34)c2)n1 ; CC1(C)OB(c2ccc3c(c2)ncc2ccc4ccccc4c23)OC1(C)C ; c1ccc(-c2nc(-c3cccc(-c4cccc5oc6ccccc6c45)c3)nc(-c3ccc4c(c3)ncc3ccc5ccccc5c34)n2)cc1]

Trz7 (15 g, 34.6 mmol) and sub2-7 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.3 g of Compound 19. (Yield: 66%, MS:[M+H]⁺=627)

### Synthesis Example 20

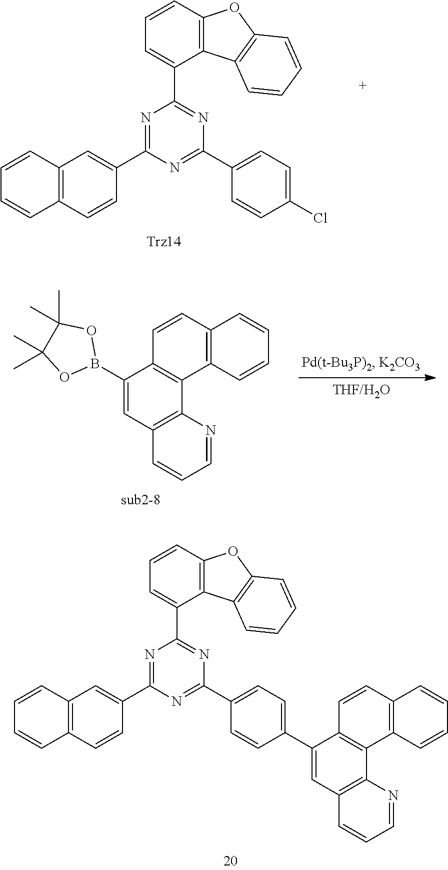 [SMILES: Clc1ccc(-c2nc(-c3ccc4ccccc4c3)nc(-c3cccc4oc5ccccc5c34)n2)cc1 ; CC1(C)OB(c2cc3cccnc3c3c2ccc2ccccc23)OC1(C)C ; c1ccc2cc(-c3nc(-c4ccc(-c5cc6cccnc6c6c5ccc5ccccc56)cc4)nc(-c4cccc5oc6ccccc6c45)n3)ccc2c1]

Trz14 (15 g, 31 mmol) and sub2-8 (11.6 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound 20. (Yield: 66%, MS:[M+H]⁺=677)

### Synthesis Example 21

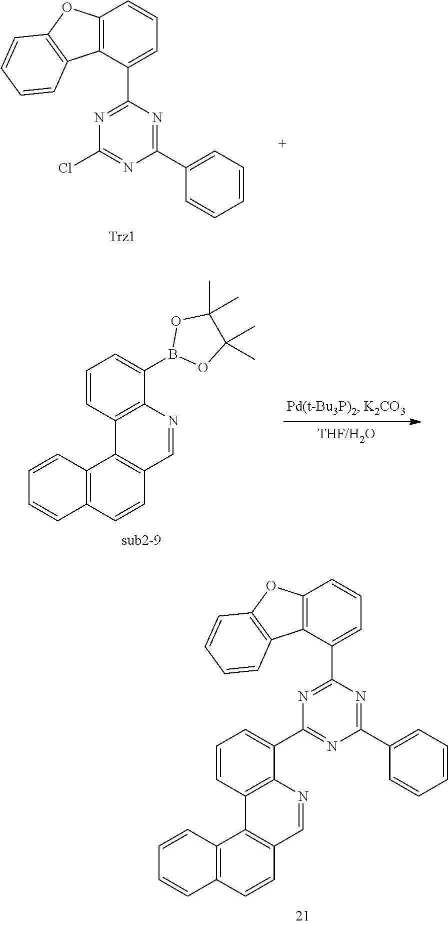 [SMILES: Clc1nc(-c2ccccc2)nc(-c2cccc3oc4ccccc4c23)n1 ; CC1(C)OB(c2cccc3c2ncc2ccc4ccccc4c23)OC1(C)C ; c1ccc(-c2nc(-c3cccc4c3ncc3ccc5ccccc5c34)nc(-c3cccc4oc5ccccc5c34)n2)cc1]

Trz1 (15 g, 41.9 mmol) and sub2-9 (15.6 g, 44 mmol) were added to 300 ml of THF, and the mixture was stirred and refluxed. Then, potassium carbonate (17.4 g, 125.8 mmol) was dissolved in 100 ml of water and added thereto, and the mixture was sufficiently stirred and then bis(tri-tert-butylphosphine)palladium(0) (0.2 g, 0.4 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15 g of Compound 21. (Yield: 65%, MS:[M+H]⁺=551)

### Synthesis Example 22

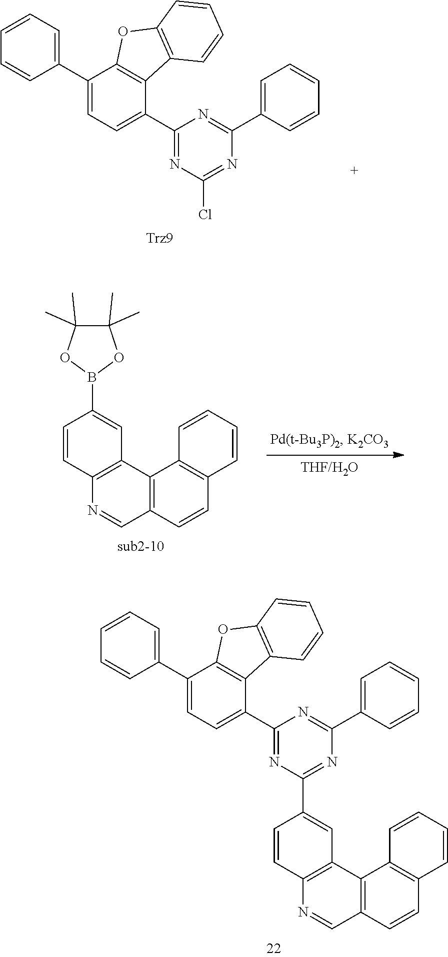 [SMILES: Clc1nc(-c2ccccc2)nc(-c2ccc(-c3ccccc3)c3oc4ccccc4c23)n1 ; CC1(C)OB(c2ccc3ncc4ccc5ccccc5c4c3c2)OC1(C)C ; c1ccc(-c2nc(-c3ccc4ncc5ccc6ccccc6c5c4c3)nc(-c3ccc(-c4ccccc4)c4oc5ccccc5c34)n2)cc1]

Trz9 (15 g, 34.6 mmol) and sub2-10 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.8 g of Compound 22. (Yield: 73%, MS:[M+H]⁺=627)

### Synthesis Example 23

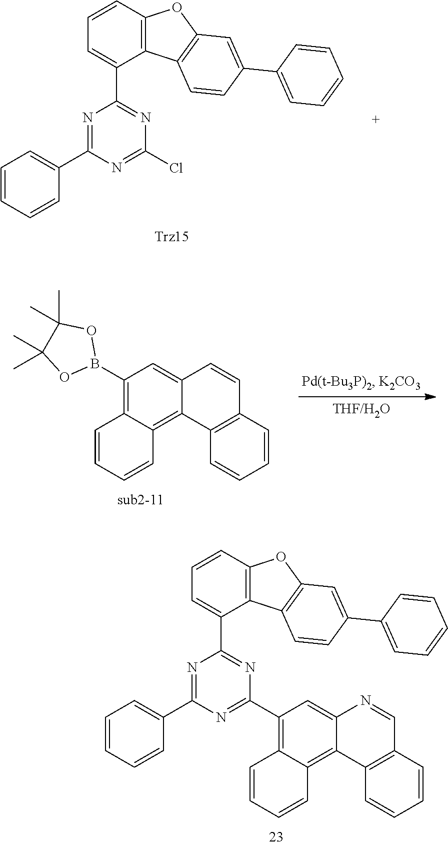 [SMILES: Clc1nc(-c2ccccc2)nc(-c2cccc3oc4cc(-c5ccccc5)ccc4c23)n1 ; CC1(C)OB(c2cc3ccc4ccccc4c3c3ccccc23)OC1(C)C ; c1ccc(-c2ccc3c(c2)oc2cccc(-c4nc(-c5ccccc5)nc(-c5cc6ncc7ccccc7c6c6ccccc56)n4)c23)cc1]

Trz15 (15 g, 34.6 mmol) and sub2-11 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.1 g of Compound 23. (Yield: 65%, MS:[M+H]⁺=627)

### Synthesis Example 24

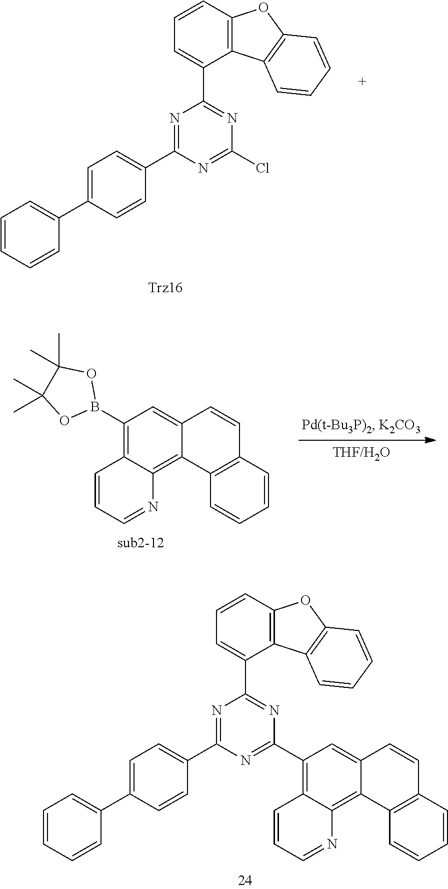 [SMILES: Clc1nc(-c2ccc(-c3ccccc3)cc2)nc(-c2cccc3oc4ccccc4c23)n1 ; CC1(C)OB(c2cc3ccc4ccccc4c3c3ncccc23)OC1(C)C ; c1ccc(-c2ccc(-c3nc(-c4cc5ccc6ccccc6c5c5ncccc45)nc(-c4cccc5oc6ccccc6c45)n3)cc2)cc1]

Trz16 (15 g, 34.6 mmol) and sub2-12 (12.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16 g of Compound 24. (Yield: 74%, MS:[M+H]⁺=627)

### Synthesis Example 25

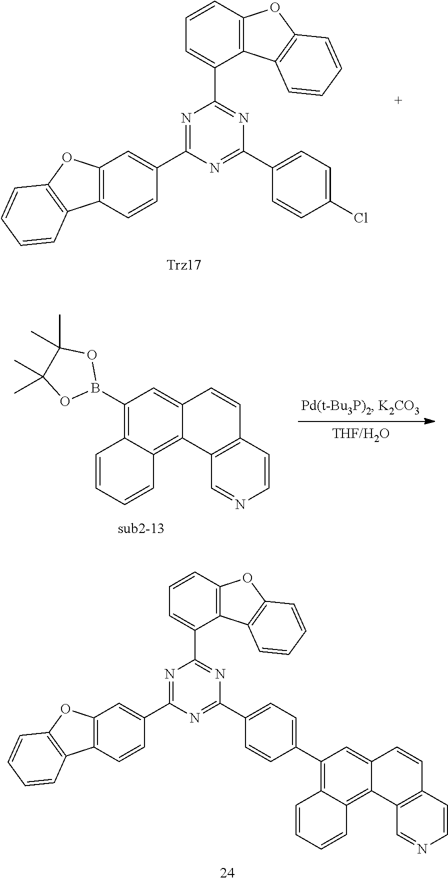 [SMILES: Clc1ccc(-c2nc(-c3ccc4c(c3)oc3ccccc34)nc(-c3cccc4oc5ccccc5c34)n2)cc1 ; CC1(C)OB(c2cc3ccc4ccncc4c3c3ccccc23)OC1(C)C ; c1ccc2c(c1)oc1cc(-c3nc(-c4ccc(-c5cc6ccc7ccncc7c6c6ccccc56)cc4)nc(-c4cccc5oc6ccccc6c45)n3)ccc12]

Trz17 (15 g, 28.6 mmol) and sub2-13 (10.7 g, 30.1 mmol) were added to 300 ml of THF, and the mixture was stirred and refluxed. Then, potassium carbonate (11.9 g, 85.9 mmol) was dissolved in 100 ml of water and added thereto, and the mixture was sufficiently stirred and then bis(tri-tert-butylphosphine)palladium(0) (0.1 g, 0.3 mmol) was added. After reacting for 5 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 16.4 g of Compound 25. (Yield: 80%, MS:[M+H]⁺=717)

### Synthesis Example 26

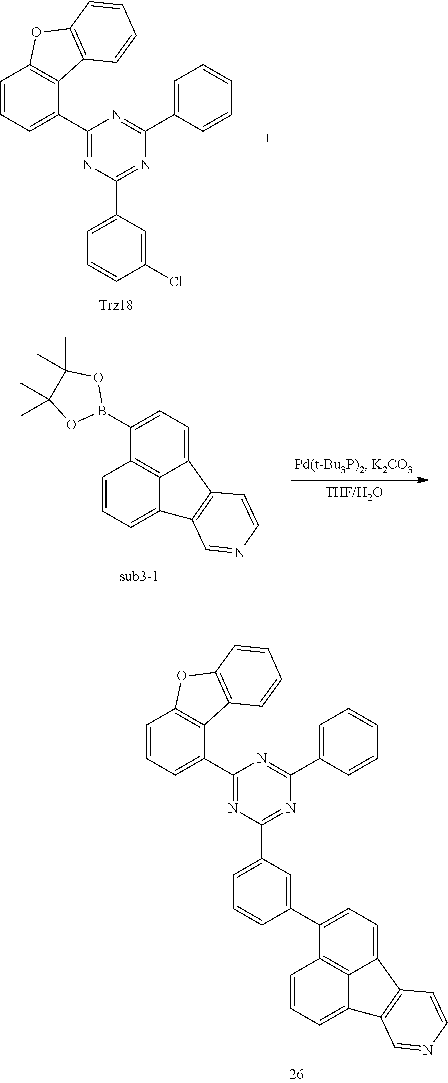 [SMILES: Clc1cccc(-c2nc(-c3ccccc3)nc(-c3cccc4oc5ccccc5c34)n2)c1 ; CC1(C)OB(c2ccc3c4c(cccc24)-c2cnccc2-3)OC1(C)C ; c1ccc(-c2nc(-c3cccc(-c4ccc5c6c(cccc46)-c4cnccc4-5)c3)nc(-c3cccc4oc5ccccc5c34)n2)cc1]

Trz18 (15 g, 34.6 mmol) and sub3-1 (11.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.7 g of Compound 26. (Yield: 61%, MS:[M+H]⁺=601)

### Synthesis Example 27

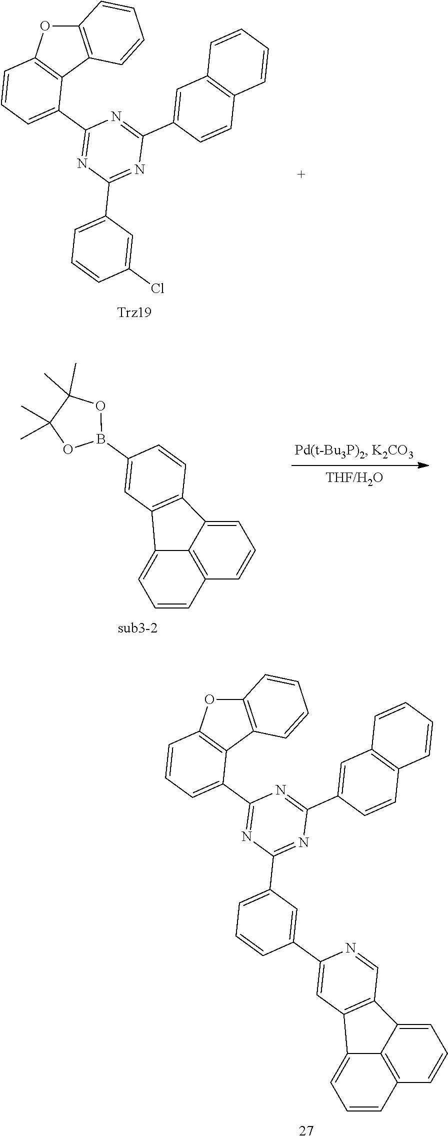 [SMILES: Clc1cccc(-c2nc(-c3ccc4ccccc4c3)nc(-c3cccc4oc5ccccc5c34)n2)c1 ; CC1(C)OB(c2ccc3c(c2)-c2cccc4cccc-3c24)OC1(C)C ; c1cc(-c2cc3c(cn2)-c2cccc4cccc-3c24)cc(-c2nc(-c3ccc4ccccc4c3)nc(-c3cccc4oc5ccccc5c34)n2)c1]

Trz19 (15 g, 31 mmol) and sub3-2 (10.7 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.3 g of Compound 27. (Yield: 71%, MS:[M+H]⁺=651)

### Synthesis Example 28

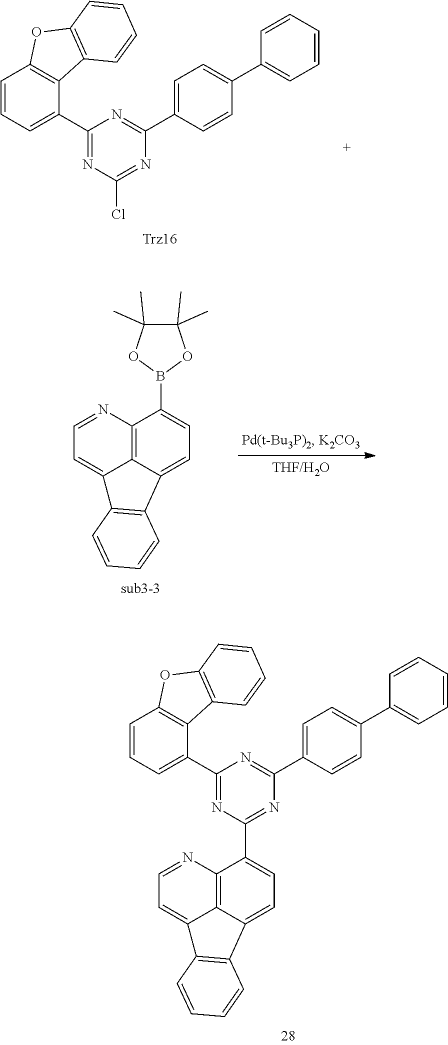 [SMILES: Clc1nc(-c2ccc(-c3ccccc3)cc2)nc(-c2cccc3oc4ccccc4c23)n1 ; CC1(C)OB(c2ccc3c4c(ccnc24)-c2ccccc2-3)OC1(C)C ; c1ccc(-c2ccc(-c3nc(-c4ccc5c6c(ccnc46)-c4ccccc4-5)nc(-c4cccc5oc6ccccc6c45)n3)cc2)cc1]

Trz16 (15 g, 34.6 mmol) and sub3-3 (11.9 g, 36.3 mmol) were added to 300 ml of THF, and the mixture was stirred and refluxed. Then, potassium carbonate (14.3 g, 103.7 mmol) was dissolved in 100 ml of water and added thereto, and the mixture was sufficiently stirred and then bis(tri-tert-butylphosphine)palladium(0) (0.2 g, 0.3 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.1 g of Compound 28. (Yield: 73%, MS:[M+H]⁺=601)

### Synthesis Example 29

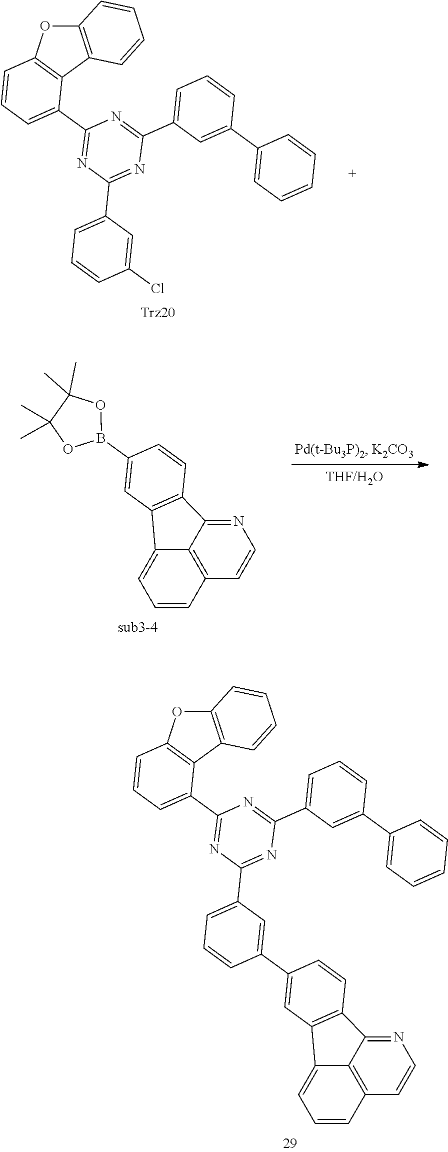 [SMILES: Clc1cccc(-c2nc(-c3cccc(-c4ccccc4)c3)nc(-c3cccc4oc5ccccc5c34)n2)c1 ; CC1(C)OB(c2ccc3c(c2)-c2cccc4ccnc-3c24)OC1(C)C ; c1ccc(-c2cccc(-c3nc(-c4cccc(-c5ccc6c(c5)-c5cccc7ccnc-6c57)c4)nc(-c4cccc5oc6ccccc6c45)n3)c2)cc1]

Trz20 (15 g, 29.4 mmol) and sub3-4 (10.2 g, 30.9 mmol) were added to 300 ml of THF, and the mixture was stirred and refluxed. Then, potassium carbonate (12.2 g, 88.2 mmol) was dissolved in 100 ml of water and added thereto, and the mixture was sufficiently stirred and then bis(tri-tert-butylphosphine)palladium(0) (0.2 g, 0.3 mmol) was added. After reacting for 2 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.5 g of Compound 29. (Yield: 73%, MS:[M+H]⁺=677)

### Synthesis Example 30

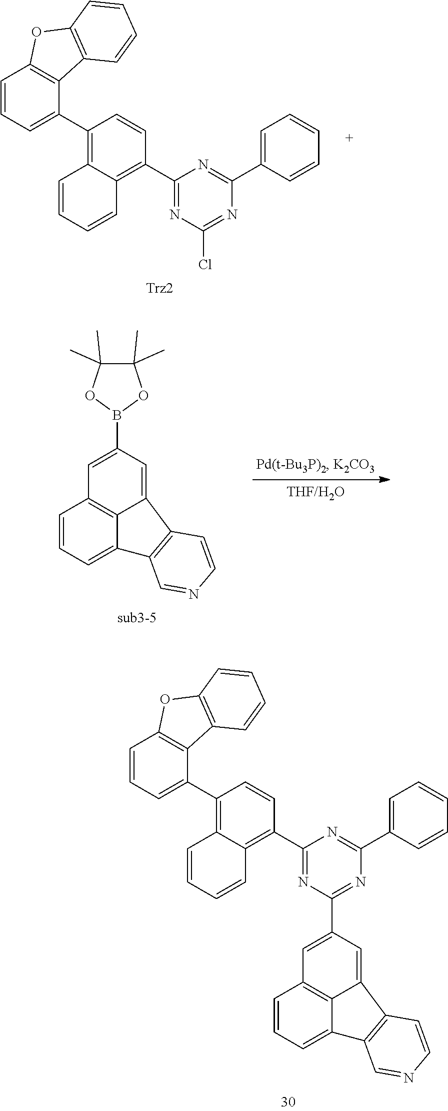 [SMILES: Clc1nc(-c2ccccc2)nc(-c2ccc(-c3cccc4oc5ccccc5c34)c3ccccc23)n1 ; CC1(C)OB(c2cc3c4c(cccc4c2)-c2cnccc2-3)OC1(C)C ; c1ccc(-c2nc(-c3cc4c5c(cccc5c3)-c3cnccc3-4)nc(-c3ccc(-c4cccc5oc6ccccc6c45)c4ccccc34)n2)cc1]

Trz2 (15 g, 31 mmol) and sub3-5 (10.7 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 4 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 14.1 g of Compound 30. (Yield: 70%, MS:[M+H]⁺=651)

### Synthesis Example 31

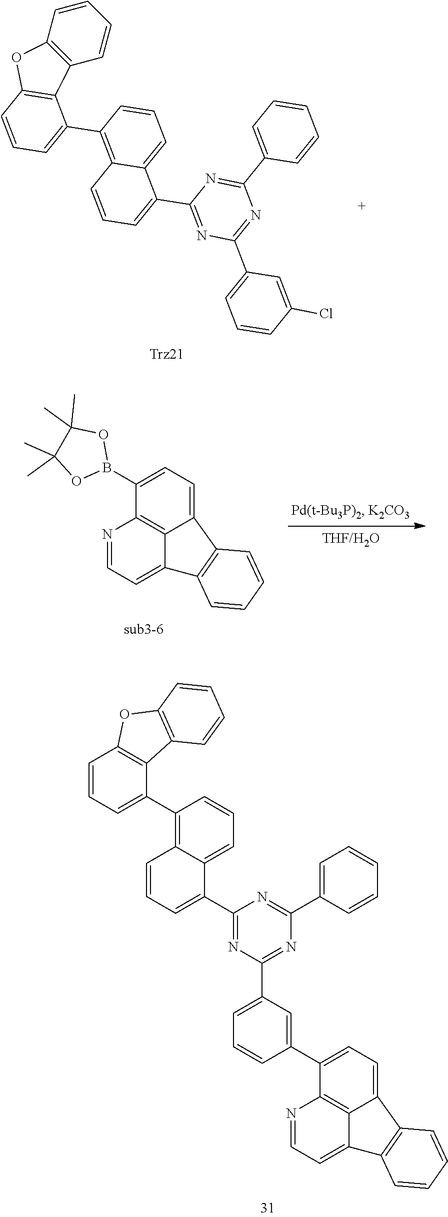 [SMILES: Clc1cccc(-c2nc(-c3ccccc3)nc(-c3cccc4c(-c5cccc6oc7ccccc7c56)cccc34)n2)c1 ; CC1(C)OB(c2ccc3c4c(ccnc24)-c2ccccc2-3)OC1(C)C ; c1ccc(-c2nc(-c3cccc(-c4ccc5c6c(ccnc46)-c4ccccc4-5)c3)nc(-c3cccc4c(-c5cccc6oc7ccccc7c56)cccc34)n2)cc1]

Trz21 (15 g, 26.8 mmol) and sub3-6 (9.3 g, 28.1 mmol) were added to 300 ml of THF, and the mixture was stirred and refluxed. Then, potassium carbonate (11.1 g, 80.3 mmol) was dissolved in 100 ml of water and added thereto, and the mixture was sufficiently stirred and then bis(tri-tert-butylphosphine)palladium(0) (0.1 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 13.8 g of Compound 31. (Yield: 71%, MS:[M+H]⁺=727)

### Synthesis Example 32

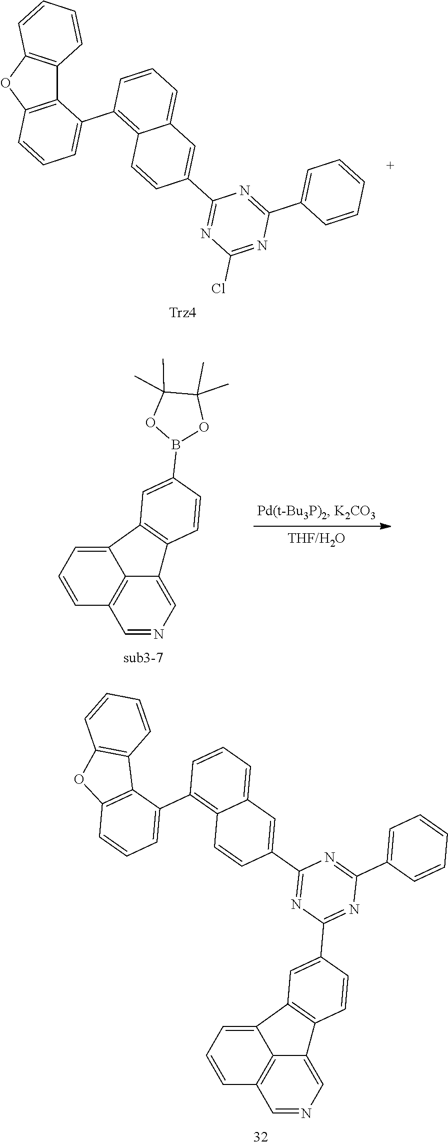 [SMILES: Clc1nc(-c2ccccc2)nc(-c2ccc3c(-c4cccc5oc6ccccc6c45)cccc3c2)n1 ; CC1(C)OB(c2ccc3c(c2)-c2cccc4cncc-3c24)OC1(C)C ; c1ccc(-c2nc(-c3ccc4c(c3)-c3cccc5cncc-4c35)nc(-c3ccc4c(-c5cccc6oc7ccccc7c56)cccc4c3)n2)cc1]

Trz4 (15 g, 31 mmol) and sub3-7 (10.7 g, 32.5 mmol) were added to 300 ml of THF, and the mixture was stirred and refluxed. Then, potassium carbonate (12.9 g, 93 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.3 g of Compound 32. (Yield: 61%, MS:[M+H]⁺=651)

### Synthesis Example 33

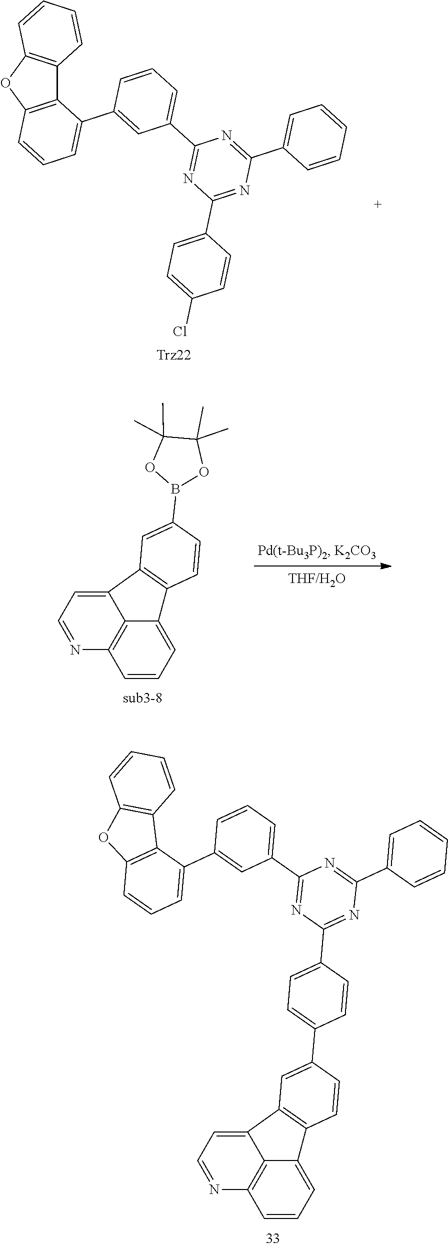 [SMILES: Clc1ccc(-c2nc(-c3ccccc3)nc(-c3cccc(-c4cccc5oc6ccccc6c45)c3)n2)cc1 ; CC1(C)OB(c2ccc3c(c2)-c2ccnc4cccc-3c24)OC1(C)C ; c1ccc(-c2nc(-c3ccc(-c4ccc5c(c4)-c4ccnc6cccc-5c46)cc3)nc(-c3cccc(-c4cccc5oc6ccccc6c45)c3)n2)cc1]

Trz22 (15 g, 29.4 mmol) and sub3-8 (10.2 g, 30.9 mmol) were added to 300 ml of THF, and the mixture was stirred and refluxed. Then, potassium carbonate (12.2 g, 88.2 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 15.7 g of Compound 33. (Yield: 79%, MS:[M+H]⁺=677)

### Synthesis Example 34

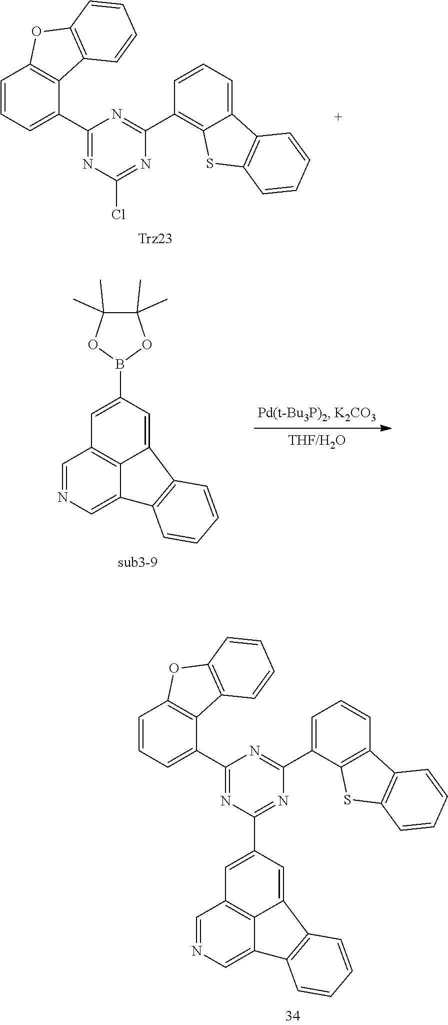 [SMILES: Clc1nc(-c2cccc3c2sc2ccccc23)nc(-c2cccc3oc4ccccc4c23)n1 ; CC1(C)OB(c2cc3c4c(cncc4c2)-c2ccccc2-3)OC1(C)C ; c1ccc2c(c1)-c1cncc3cc(-c4nc(-c5cccc6c5sc5ccccc56)nc(-c5cccc6oc7ccccc7c56)n4)cc-2c13]

Trz23 (15 g, 32.3 mmol) and sub3-9 (11.2 g, 33.9 mmol) were added to 300 ml of THF, and the mixture was stirred and refluxed. Then, potassium carbonate (13.4 g, 97 mmol) was dissolved in 100 ml of water and added thereto, and the mixture was sufficiently stirred and then bis(tri-tert-butylphosphine)palladium(0) (0.2 g, 0.3 mmol) was added. After reacting for 3 hours, the reaction mixture was cooled to room temperature, and then the organic layer and the aqueous layer were separated, and the organic layer was distilled. This was again dissolved in chloroform, washed twice with water, and then the organic layer was separated, anhydrous magnesium sulfate was added, stirred, then filtered, and the filtrate was distilled under reduced pressure. The concentrated compound was purified by a silica gel column chromatography to prepare 12.8 g of Compound 34. (Yield: 63%, MS:[M+H]⁺=631)

### Comparative Example 1

A glass substrate on which a thin film of ITO (indium tin oxide) was coated in a thickness of 1,000 Å was put into distilled water containing a detergent dissolved therein and ultrasonically cleaned. In this case, the detergent used was a product commercially available from Fischer Co. and the distilled water was one which had been twice filtered by using a filter commercially available from Millipore Co. The ITO was cleaned for 30 minutes, and ultrasonic cleaning was then repeated twice for 10 minutes by using distilled water. After the cleaning with distilled water was completed, the substrate was ultrasonically washed with the solvents of isopropyl alcohol, acetone, and methanol, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, wherein the following compound A-1 was p-doped at a concentration of 1.5 wt. %. The following compound HT-1 was vacuum deposited on the hole injection layer to form a hole transport layer with a layer thickness of 800 Å. Then, the following compound EB-1 was vacuum deposited on the hole transport layer to a layer thickness of 150 Å to form an electron blocking layer. Then, the following compound RH-1 and the following compound Dp-39 were vacuum deposited at a weight ratio of 98:2 on the EB-1 deposited layer to form a red light emitting layer with a layer thickness of 400 Å. The following compound HB-1 was vacuum deposited on the light emitting layer to a layer thickness of 30 Å to form a hole blocking layer. Then, the following compound ET-1 and the following compound LiQ were vacuum deposited in a weight ratio of 2:1 on the hole blocking layer to form an electron injection and transport layer with a layer thickness of 300 Å. Lithium fluoride (LiF) and aluminum were sequentially deposited to have a thickness of 12 Å and 1,000 Å, respectively, on the electron injection and transport layer, thereby forming a cathode.

In the above-mentioned processes, the deposition rates of the organic materials were maintained at 0.4˜0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

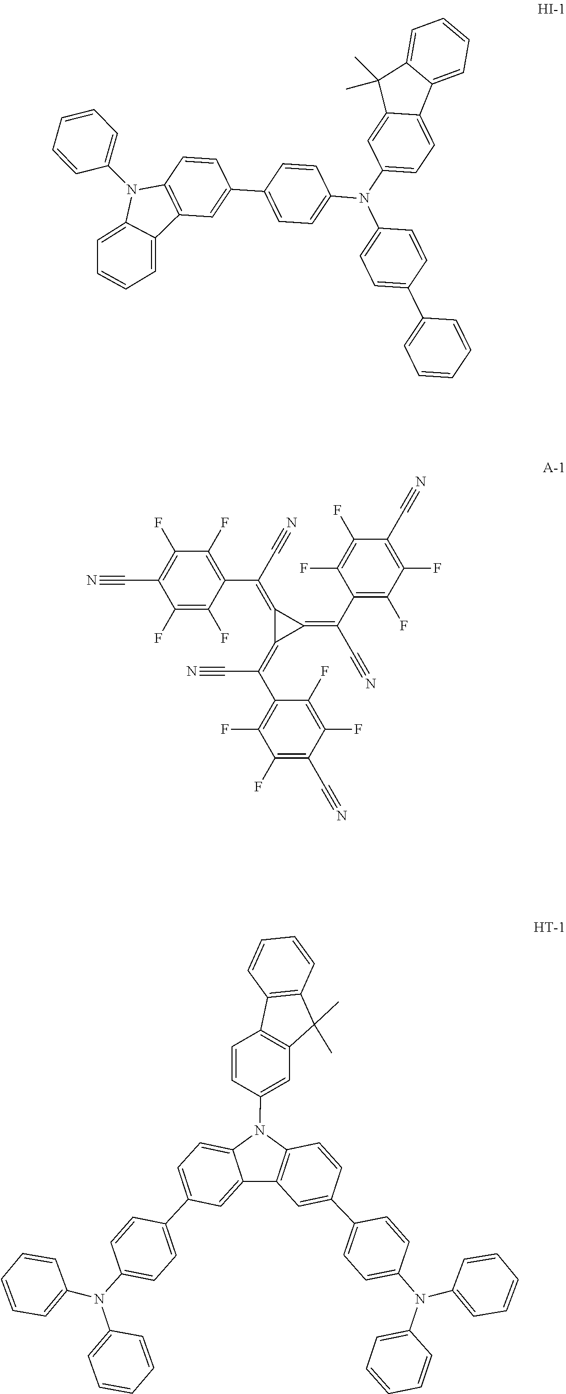 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(-n3c4ccc(-c5ccc(N(c6ccccc6)c6ccccc6)cc5)cc4c4cc(-c5ccc(N(c6ccccc6)c6ccccc6)cc5)ccc43)cc21]

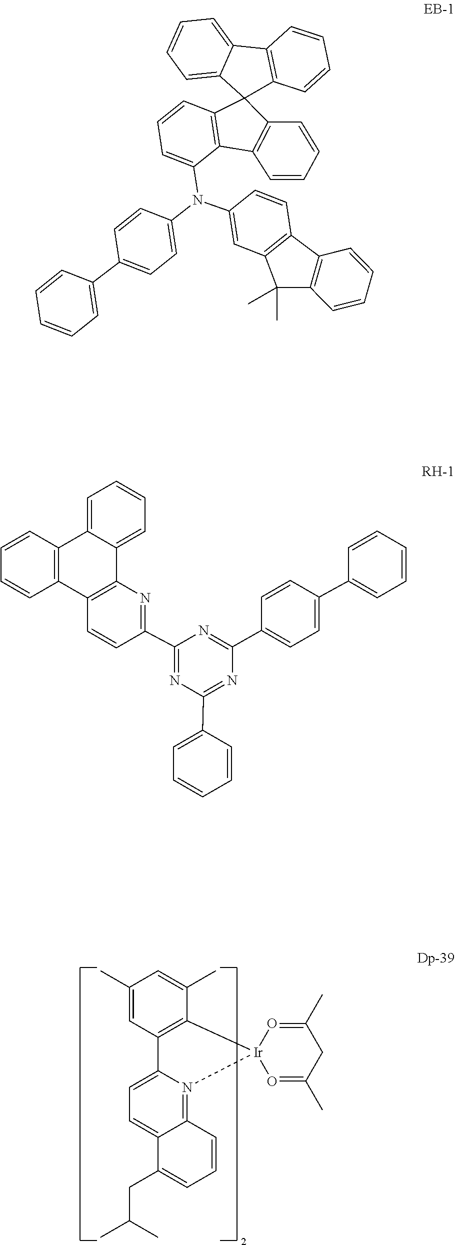

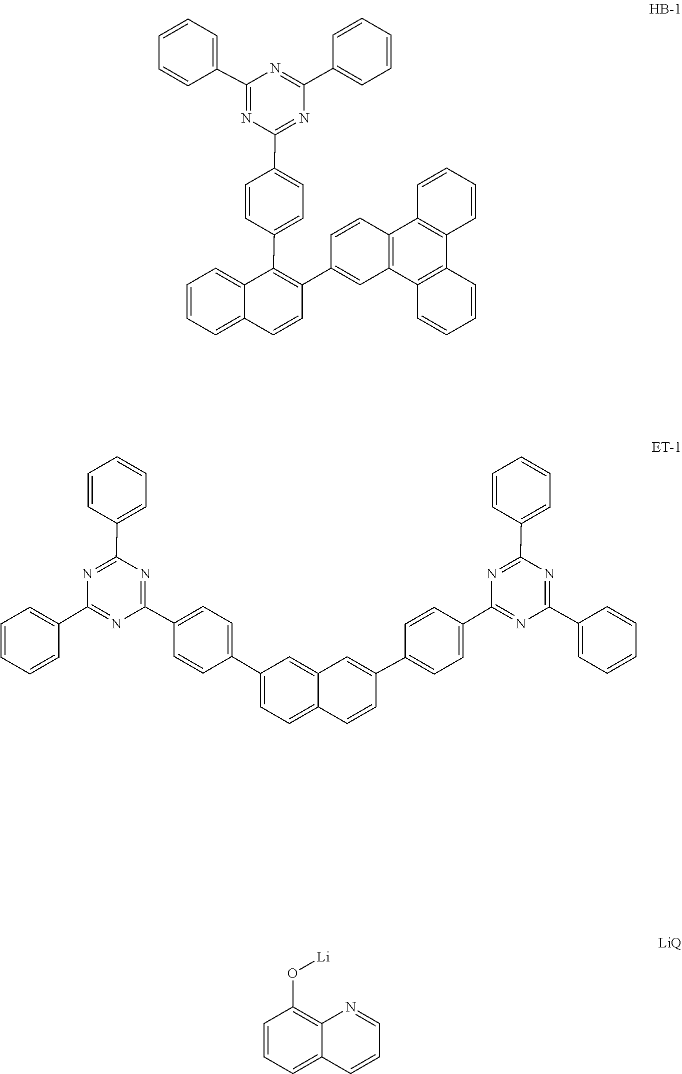

### Comparative Examples 2 to 6

The procedure was performed in the same manner as in Comparative Example 1, except that the following compounds RH-2 to RH-6 were respectively used instead of RH-1 as the host material for the red light emitting layer, and the device performance was measured.

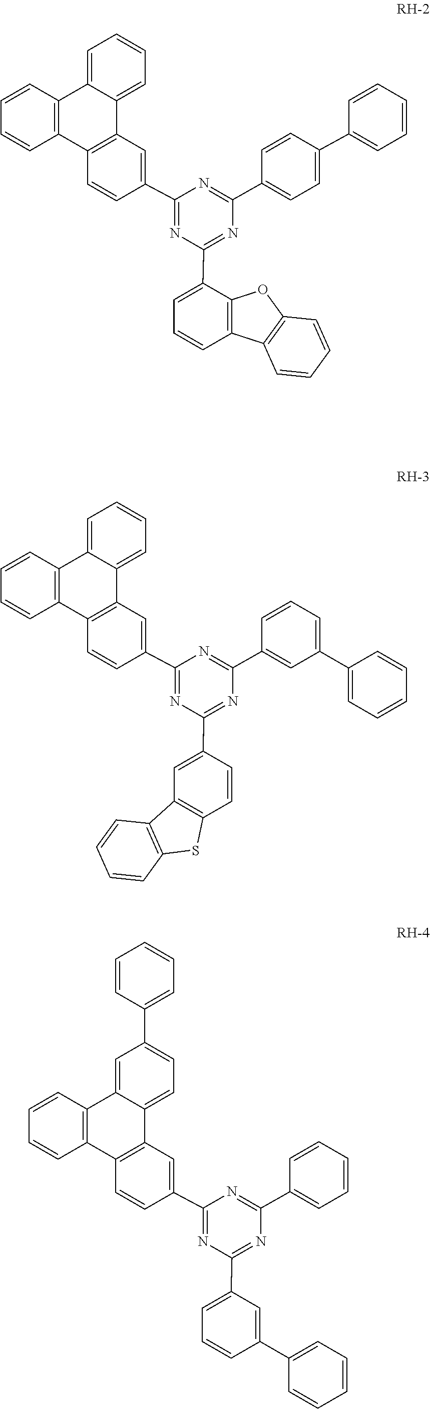 [SMILES: c1ccc(-c2ccc(-c3nc(-c4ccc5c6ccccc6c6ccccc6c5c4)nc(-c4cccc5c4oc4ccccc45)n3)cc2)cc1 ; c1ccc(-c2cccc(-c3nc(-c4ccc5sc6ccccc6c5c4)nc(-c4ccc5c6ccccc6c6ccccc6c5c4)n3)c2)cc1 ; c1ccc(-c2cccc(-c3nc(-c4ccccc4)nc(-c4ccc5c6ccccc6c6cc(-c7ccccc7)ccc6c5c4)n3)c2)cc1]

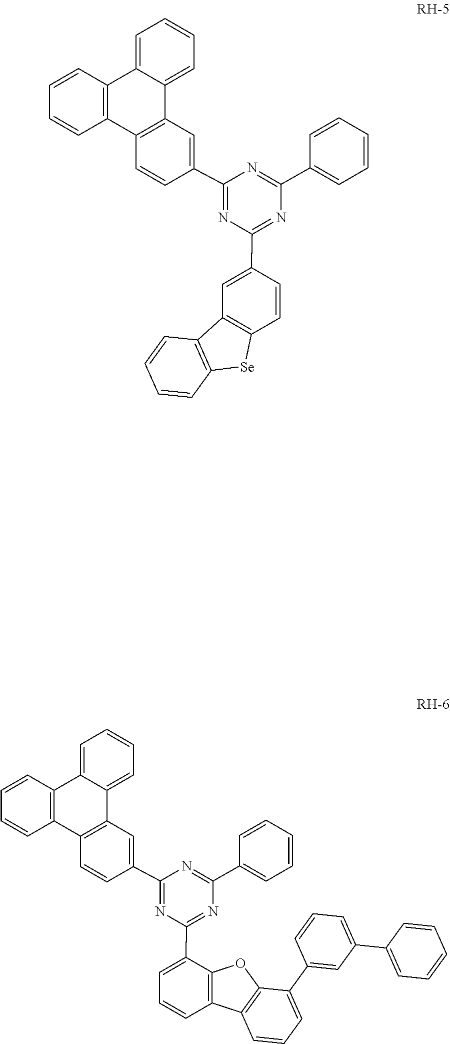 [SMILES: c1ccc(-c2nc(-c3ccc4[se]c5ccccc5c4c3)nc(-c3ccc4c5ccccc5c5ccccc5c4c3)n2)cc1 ; c1ccc(-c2cccc(-c3cccc4c3oc3c(-c5nc(-c6ccccc6)nc(-c6ccc7c8ccccc8c8ccccc8c7c6)n5)cccc34)c2)cc1]

### Examples 1 to 34

The procedure was performed in the same manner as in Comparative Example 1, except that Compounds 1 to 4 and 6 to 34 prepared by Synthesis Examples were respectively used instead of RH-1 as the host material for the red light emitting layer, and the device performance was measured.

The driving voltage, current efficiency and lifetime were measured for the organic light emitting devices manufactured by using the compounds as the red host materials as in Comparative Examples 1 to 6 and Examples 1 to 4 and 6 to 34, and the results are shown in Tables 1 to Table 3 below.

TABLE 1

| | | Driving | Current | Lifetime |
|---|---|---|---|---|
| Experimental | Host | voltage | efficiency | (T95% |
| Example | material | (V) | (cd/A) | @10 mA) |
| Comparative | RH-1 | 5.68 | 14.33 | 59 |
| Example 1 | | | | |
| Comparative | RH-2 | 5.69 | 13.95 | 58 |
| Example 2 | | | | |
| Comparative | RH-3 | 5.71 | 13.86 | 60 |
| Example 3 | | | | |
| Comparative | RH-4 | 5.89 | 13.71 | 57 |
| Example 4 | | | | |
| Comparative | RH-5 | 5.75 | 12.73 | 69 |
| Example 5 | | | | |
| Comparative | RH-6 | 5.80 | 12.97 | 61 |
| Example 6 | | | | |

TABLE 2

| | | Driving | Current | Lifetime |
|---|---|---|---|---|
| Experimental | Host | voltage | efficiency | (T95% |
| Example | material | (V) | (cd/A) | @10 mA) |
| Example 1 | Compound 1 | 4.09 | 23.55 | 179 |
| Example 2 | Compound 2 | 4.21 | 24.51 | 185 |
| Example 3 | Compound 3 | 4.10 | 24.39 | 169 |
| Example 4 | Compound 4 | 4.25 | 23.99 | 177 |
| Example 5 | Compound 5 | 4.26 | 23.79 | 171 |
| Example 6 | Compound 6 | 4.19 | 23.98 | 188 |
| Example 7 | Compound 7 | 4.20 | 24.12 | 186 |
| Example 8 | Compound 8 | 4.23 | 25.09 | 190 |
| Example 9 | Compound 9 | 4.30 | 25.01 | 178 |
| Example 10 | Compound 10 | 4.19 | 24.29 | 181 |
| Example 11 | Compound 11 | 4.41 | 24.99 | 191 |
| Example 12 | Compound 12 | 4.36 | 25.01 | 200 |
| Example 13 | Compound 13 | 4.31 | 25.19 | 191 |
| Example 14 | Compound 14 | 4.26 | 26.00 | 185 |
| Example 15 | Compound 15 | 4.29 | 24.89 | 181 |
| Example 16 | Compound 16 | 4.20 | 24.33 | 177 |
| Example 17 | Compound 17 | 4.31 | 24.25 | 193 |

TABLE 3

| | | Driving | Current | Lifetime |
|---|---|---|---|---|
| Experimental | Host | voltage | efficiency | (T95% |
| Example | material | (V) | (cd/A) | @10 mA) |
| Example 18 | Compound 18 | 4.39 | 24.20 | 181 |
| Example 19 | Compound 19 | 4.16 | 24.01 | 188 |
| Example 20 | Compound 20 | 4.24 | 24.09 | 176 |
| Example 21 | Compound 21 | 4.22 | 23.69 | 180 |
| Example 22 | Compound 22 | 4.36 | 24.37 | 183 |
| Example 23 | Compound 23 | 4.33 | 24.33 | 175 |
| Example 24 | Compound 24 | 4.29 | 23.98 | 183 |
| Example 25 | Compound 25 | 4.22 | 24.84 | 180 |
| Example 26 | Compound 26 | 4.31 | 24.67 | 178 |
| Example 27 | Compound 27 | 4.26 | 25.36 | 183 |
| Example 28 | Compound 28 | 4.18 | 24.33 | 194 |
| Example 29 | Compound 29 | 4.35 | 25.31 | 190 |
| Example 30 | Compound 30 | 4.27 | 24.87 | 189 |
| Example 31 | Compound 31 | 4.34 | 23.99 | 176 |
| Example 32 | Compound 32 | 4.39 | 23.69 | 196 |
| Example 33 | Compound 33 | 4.27 | 24.57 | 194 |
| Example 34 | Compound 34 | 4.35 | 25.06 | 200 |

[Description of Symbols]

| | |
|---|---|
| 1: substrate | 2: anode |
| 3: hole transport layer | 4: light emitting layer |
| 5: electron injection and transport layer | 6: cathode |
| 7: hole injection layer | 8: electron blocking layer |
| 9: hole blocking layer | |

1. A compound of Chemical Formula 1: 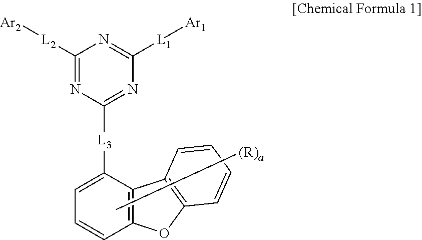
wherein, in Chemical Formula 1:
L₁ to L₃ are each independently a single bond, a substituted or unsubstituted C₆₋₆₀ arylene, or a substituted or unsubstituted C₂₋₆₀ heteroarylene containing at least one heteroatom of N, O and S;
Ar₁ and Ar₂ are each independently a substituted or unsubstituted C₆₋₆₀ aryl or a substituted or unsubstituted C₂₋₆₀ heteroaryl containing at least one heteroatom of N, O and S,
provided that Ar₁ and Ar₂ are not benzocarbazolyl, and at least one of Ar₁ and Ar₂ is a substituted or unsubstituted azachrysenyl, a substituted or unsubstituted azabenzo[c]phenanthrenyl, a substituted or unsubstituted azafluoranthenyl, a substituted or unsubstituted azatetracenyl, a substituted or unsubstituted azatetraphenyl, a substituted or unsubstituted azapyrenyl, a substituted or unsubstituted azatriphenylenyl, a substituted or unsubstituted azaaceanthrylene, a substituted or unsubstituted azaacephenanthrylene, or a substituted or unsubstituted azapleiadenyl;
R is deuterium or a substituted or unsubstituted C₆₋₆₀ aryl; and
a is an integer of 0 to 7.

2. The compound according to claim 1, wherein:
L₁ and L₂ are each independently a single bond or phenylene, and
when L₁ and L₂ are each independently phenylene, they are unsubstituted or substituted with at least one deuterium.

3. The compound according to claim 1, wherein:
L₃ is a single bond, phenylene, or naphthylene, and
when L₃ is phenylene or naphthylene, the L₃ is unsubstituted or substituted with at least one deuterium.

4. The compound according to claim 1, wherein:
Ar₁ is monoazachrysenyl, monoazabenzo[c]phenanthrenyl, monoazafluoranthenyl, monoazatetracenyl, monoazatetraphenyl, monoazapyrenyl, monoazatriphenylenyl, monoazaaceanthrylene, monoazaacephenanthrylenyl, or monoazapleiadenyl;
Ar₂ is a C₆₋₂₀ aryl or a C₂₋₂₀ heteroaryl containing at least one heteroatom of O and S; and
Ar₁ and Ar₂ are unsubstituted or substituted with at least one deuterium.

5. The compound according to claim 4, wherein:
Ar₁ is any one selected from the group consisting of the following, and
when Ar₁ is unsubstituted or substituted with at least one deuterium;

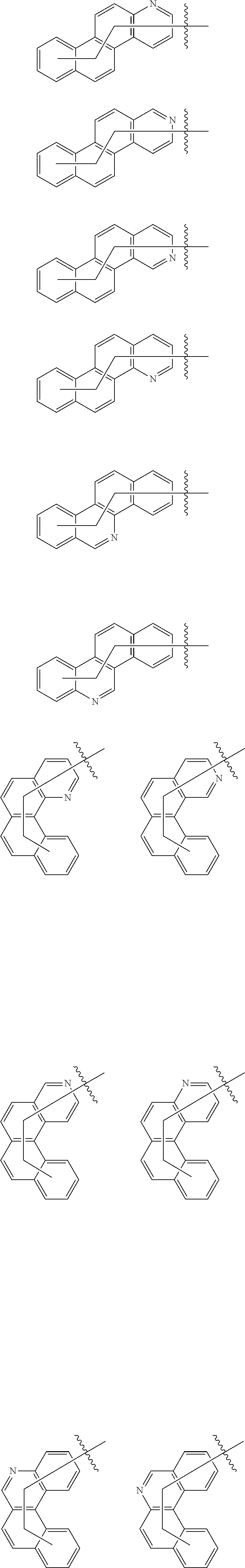

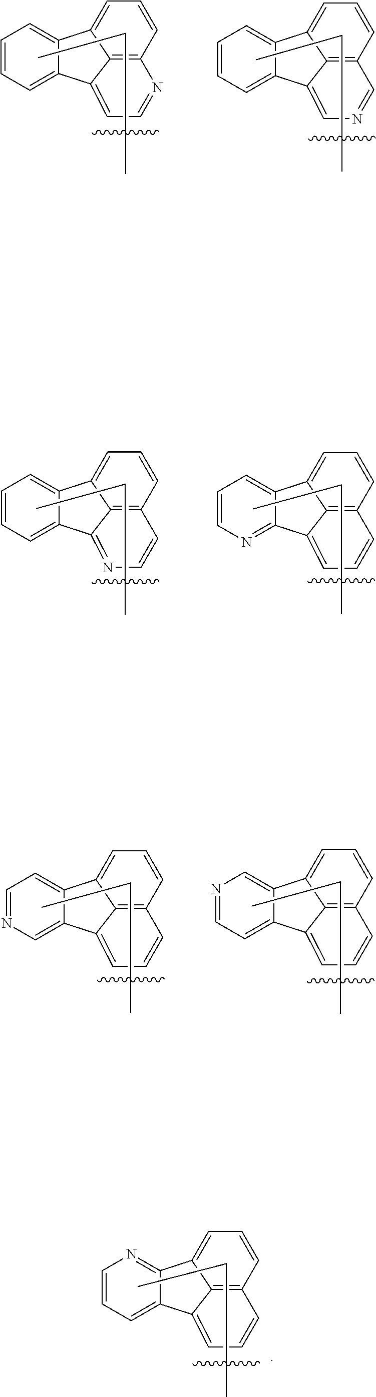

6. The compound according to claim 4, wherein:
Ar₂ is phenyl, biphenylyl, naphthyl, dibenzofuranyl, or dibenzothiophenyl; and
Ar₂ is unsubstituted or substituted with at least one deuterium.

7. The compound according to claim 1, wherein:
R is phenyl that is unsubstituted or substituted with at least one deuterium; and a is 0 or 1.

8. The compound according to claim 1, wherein:
the compound is any one of the following Chemical Formulas 1-1 to 1-3: 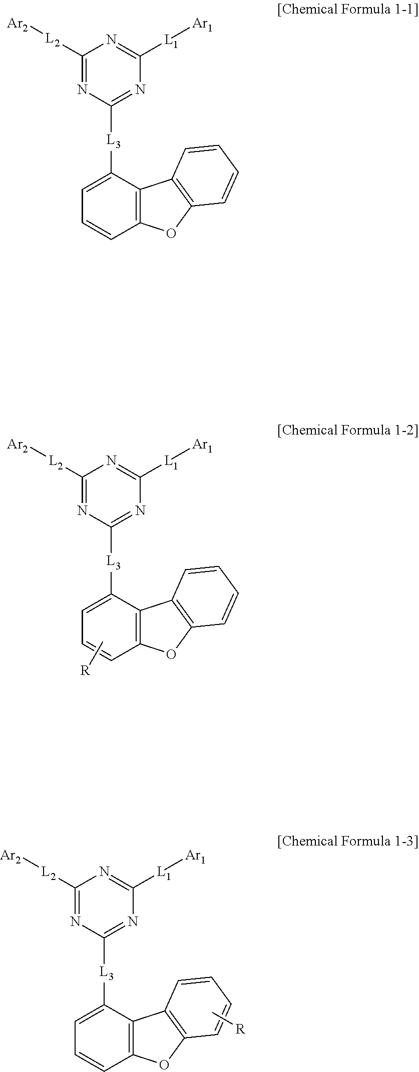
wherein, in Chemical Formulas 1-1 to 1-3:
R is a C₆₋₂₀ aryl that is unsubstituted or substituted with at least one deuterium; and
L₁ to L₃, Ar₁ and Ar₂ are as defined in claim 1.

9. The compound according to claim 1, wherein:
the compound is any one compound selected from the group consisting of the following compounds:

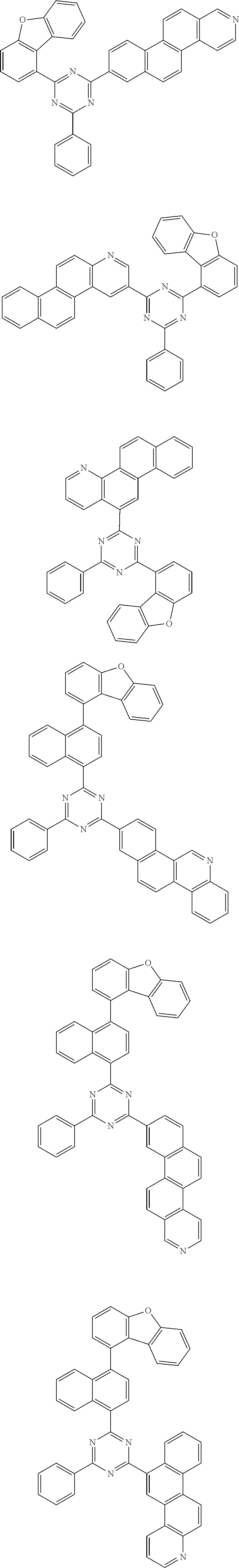

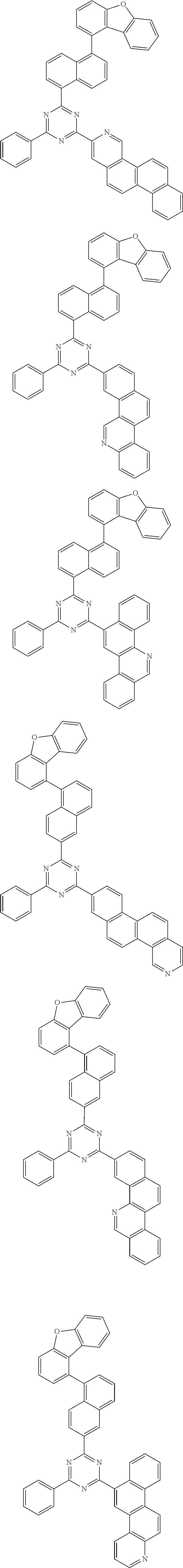

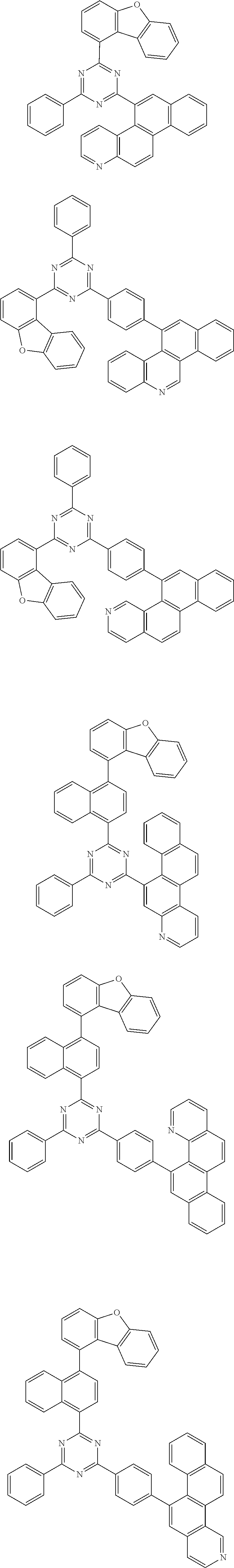

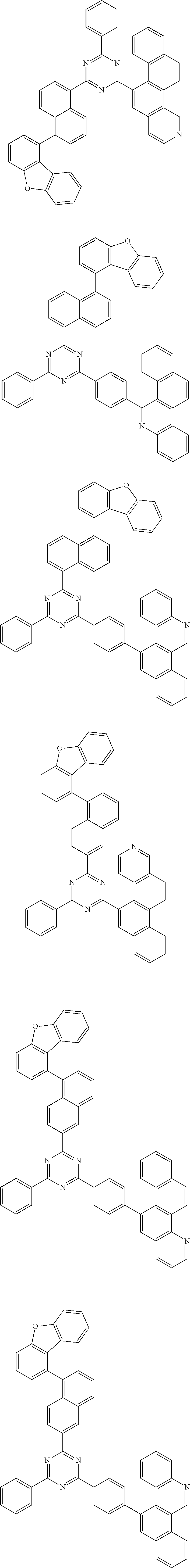

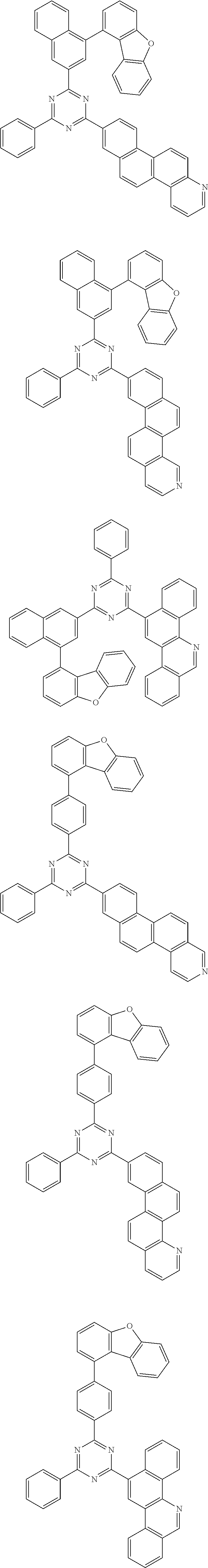

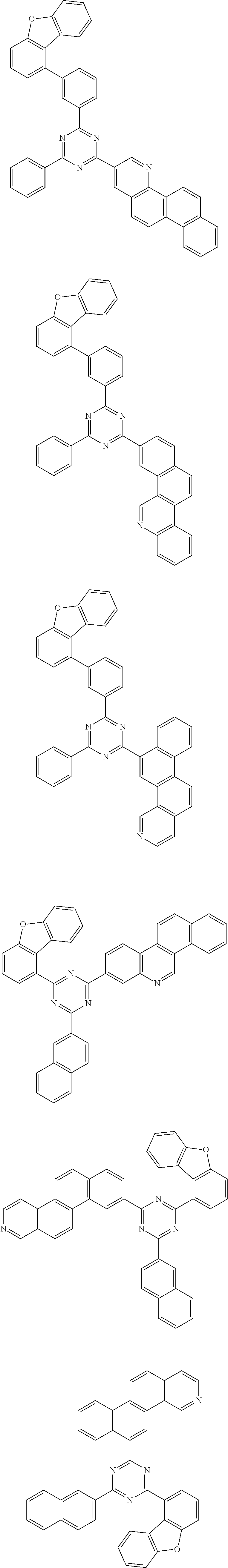 [SMILES: c1ccc(-c2nc(-c3cccc(-c4cccc5oc6ccccc6c45)c3)nc(-c3cnc4c(ccc5c6ccccc6ccc54)c3)n2)cc1 ; c1ccc(-c2nc(-c3cccc(-c4cccc5oc6ccccc6c45)c3)nc(-c3ccc4ccc5c6ccccc6ncc5c4c3)n2)cc1 ; c1ccc(-c2nc(-c3cccc(-c4cccc5oc6ccccc6c45)c3)nc(-c3cc4c5cnccc5ccc4c4ccccc34)n2)cc1 ; c1ccc2cc(-c3nc(-c4ccc5c(c4)ncc4c6ccccc6ccc54)nc(-c4cccc5oc6ccccc6c45)n3)ccc2c1 ; c1ccc2cc(-c3nc(-c4ccc5ccc6c7ccncc7ccc6c5c4)nc(-c4cccc5oc6ccccc6c45)n3)ccc2c1 ; c1ccc2cc(-c3nc(-c4cc5c6cnccc6ccc5c5ccccc45)nc(-c4cccc5oc6ccccc6c45)n3)ccc2c1]

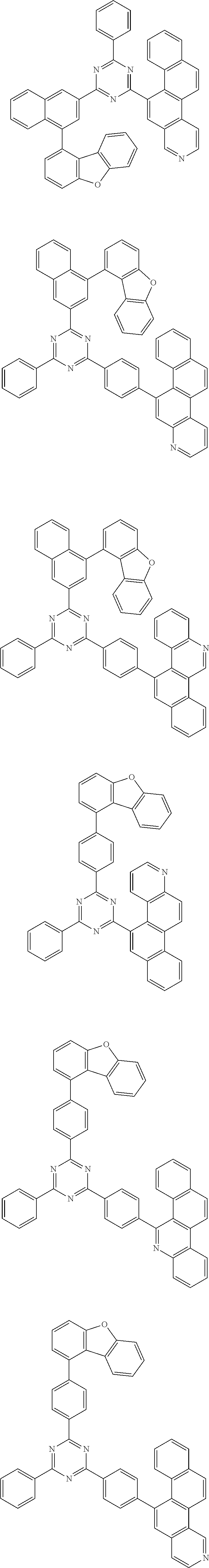

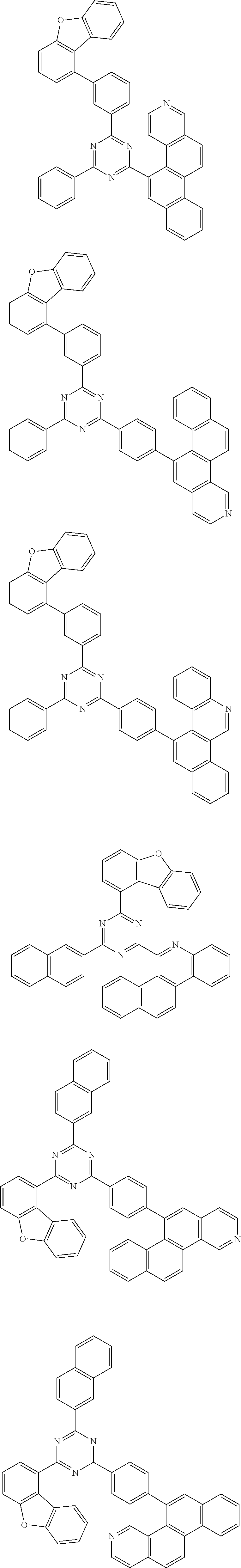

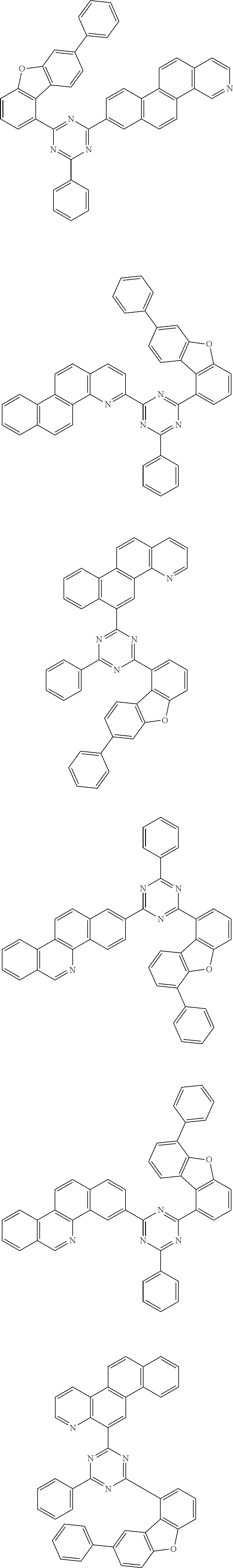

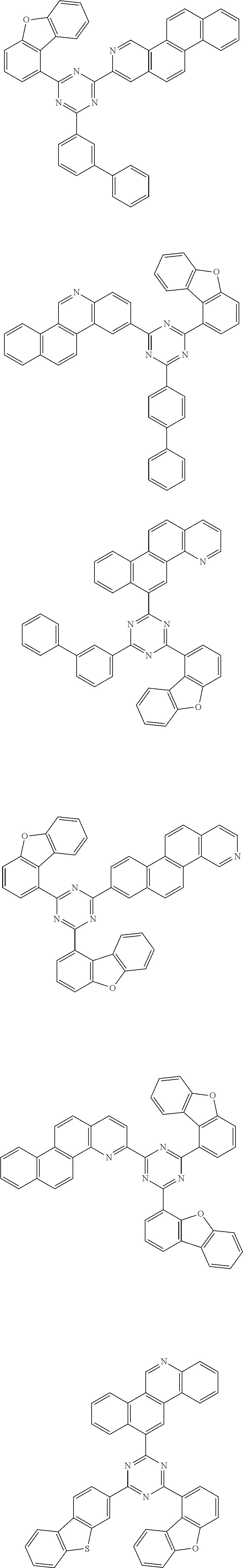

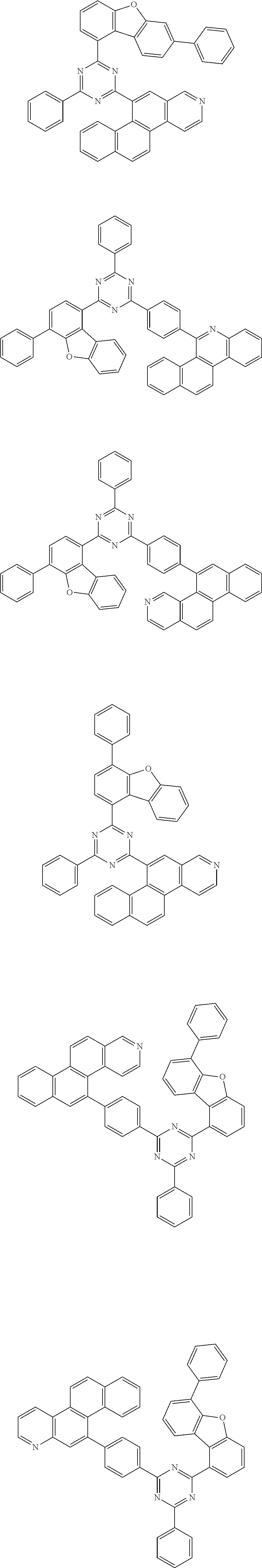

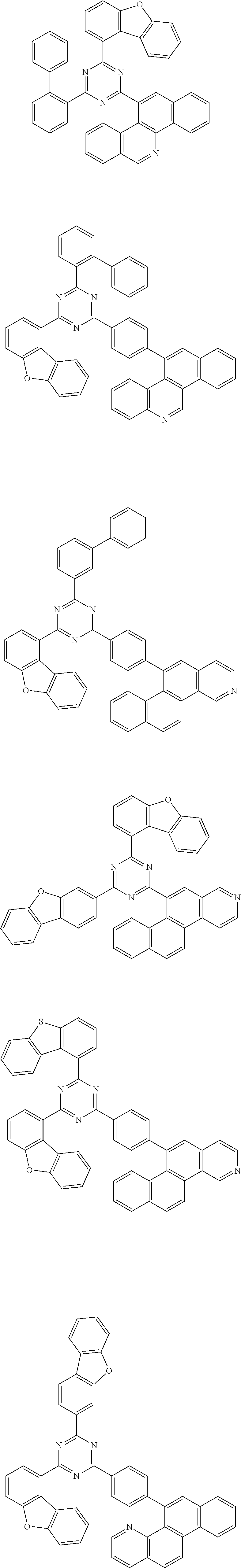

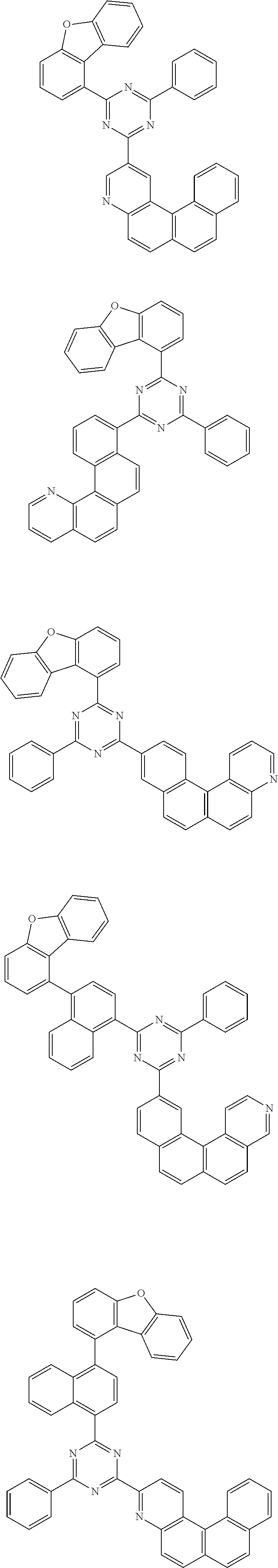 [SMILES: c1ccc(-c2nc(-c3cnc4ccc5ccc6ccccc6c5c4c3)nc(-c3cccc4oc5ccccc5c34)n2)cc1 ; c1ccc(-c2nc(-c3cccc4c3ccc3ccc5cccnc5c34)nc(-c3cccc4oc5ccccc5c34)n2)cc1 ; c1ccc(-c2nc(-c3ccc4c(ccc5ccc6ncccc6c54)c3)nc(-c3cccc4oc5ccccc5c34)n2)cc1 ; c1ccc(-c2nc(-c3ccc4ccc5ccc6cnccc6c5c4c3)nc(-c3ccc(-c4cccc5oc6ccccc6c45)c4ccccc34)n2)cc1 ; c1ccc(-c2nc(-c3ccc4c(ccc5ccc6ccccc6c54)n3)nc(-c3ccc(-c4cccc5oc6ccccc6c45)c4ccccc34)n2)cc1]

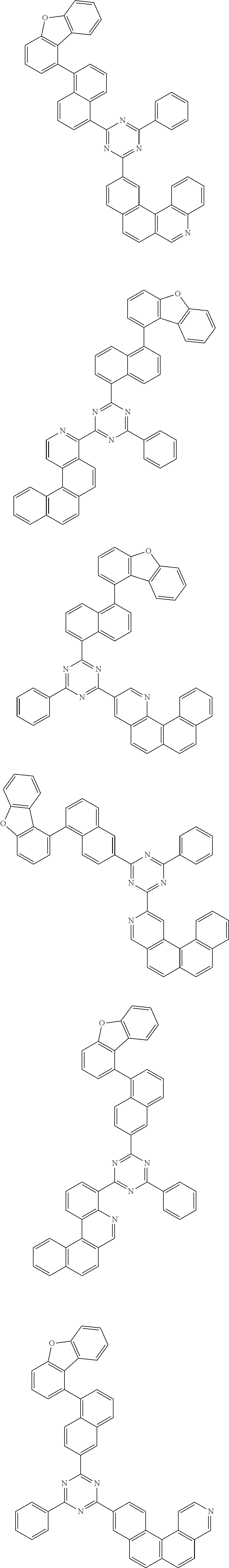

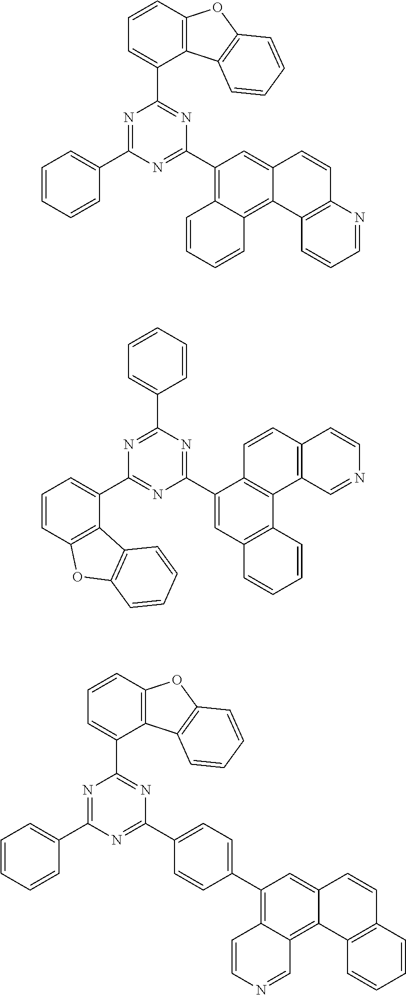 [SMILES: c1ccc(-c2nc(-c3cc4ccc5ncccc5c4c4ccccc34)nc(-c3cccc4oc5ccccc5c34)n2)cc1 ; c1ccc(-c2nc(-c3cc4ccccc4c4c3ccc3ccncc34)nc(-c3cccc4oc5ccccc5c34)n2)cc1 ; c1ccc(-c2nc(-c3ccc(-c4cc5ccc6ccccc6c5c5cnccc45)cc3)nc(-c3cccc4oc5ccccc5c34)n2)cc1]

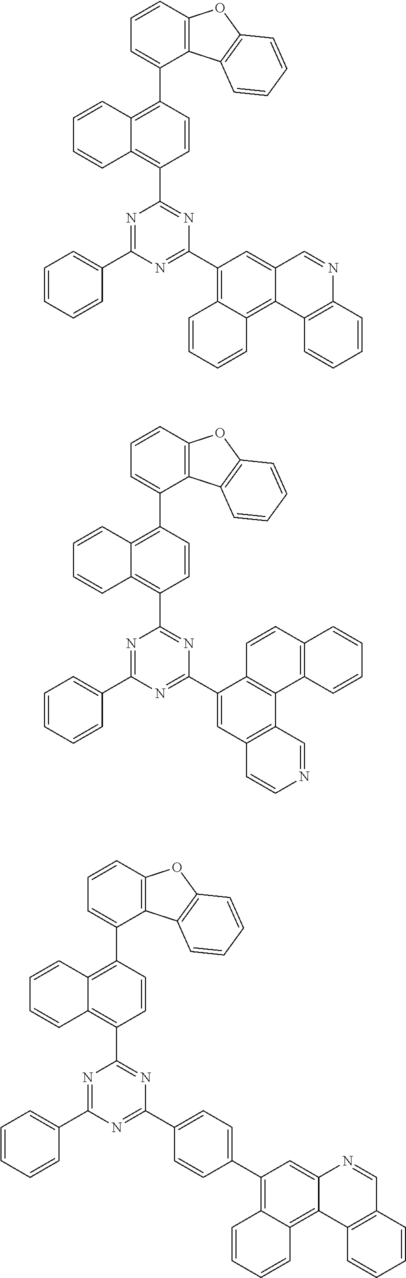 [SMILES: c1ccc(-c2nc(-c3ccc(-c4cccc5oc6ccccc6c45)c4ccccc34)nc(-c3cc4cnc5ccccc5c4c4ccccc34)n2)cc1 ; c1ccc(-c2nc(-c3ccc(-c4cccc5oc6ccccc6c45)c4ccccc34)nc(-c3cc4ccncc4c4c3ccc3ccccc34)n2)cc1 ; c1ccc(-c2nc(-c3ccc(-c4cc5ncc6ccccc6c5c5ccccc45)cc3)nc(-c3ccc(-c4cccc5oc6ccccc6c45)c4ccccc34)n2)cc1]

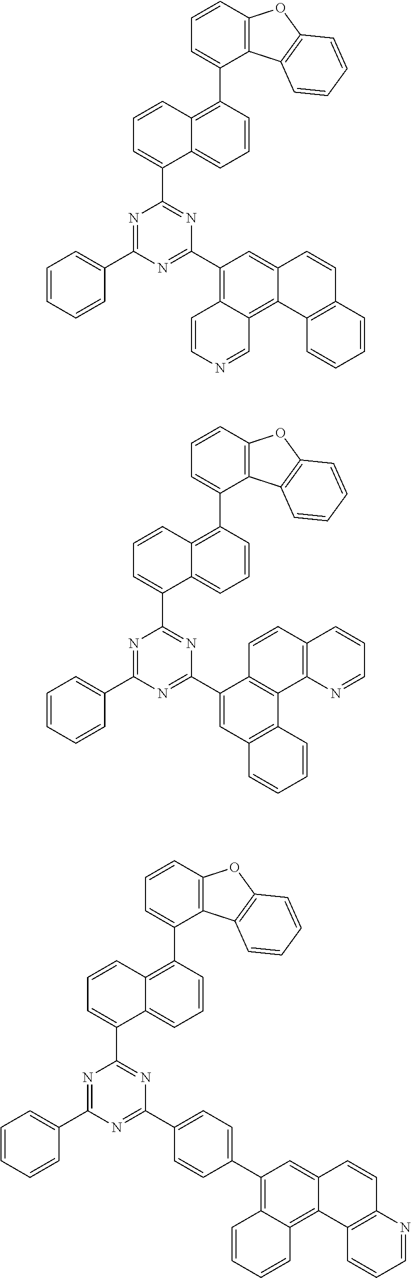 [SMILES: c1ccc(-c2nc(-c3cccc4c(-c5cccc6oc7ccccc7c56)cccc34)nc(-c3cc4ccc5ccccc5c4c4cnccc34)n2)cc1 ; c1ccc(-c2nc(-c3cccc4c(-c5cccc6oc7ccccc7c56)cccc34)nc(-c3cc4ccccc4c4c3ccc3cccnc34)n2)cc1 ; c1ccc(-c2nc(-c3ccc(-c4cc5ccc6ncccc6c5c5ccccc45)cc3)nc(-c3cccc4c(-c5cccc6oc7ccccc7c56)cccc34)n2)cc1]

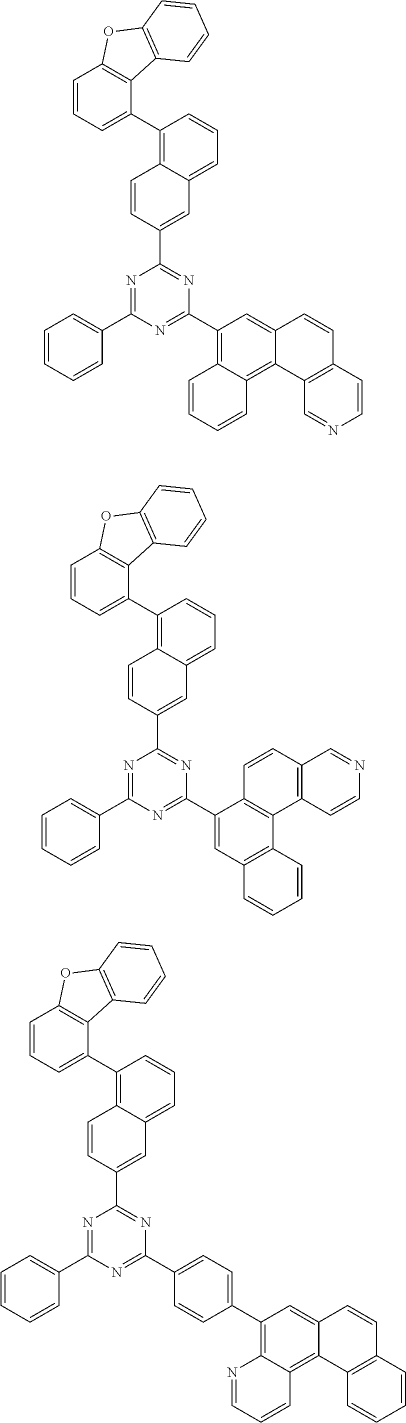 [SMILES: c1ccc(-c2nc(-c3ccc4c(-c5cccc6oc7ccccc7c56)cccc4c3)nc(-c3cc4ccc5ccncc5c4c4ccccc34)n2)cc1 ; c1ccc(-c2nc(-c3ccc4c(-c5cccc6oc7ccccc7c56)cccc4c3)nc(-c3cc4ccccc4c4c3ccc3cnccc34)n2)cc1 ; c1ccc(-c2nc(-c3ccc(-c4cc5ccc6ccccc6c5c5cccnc45)cc3)nc(-c3ccc4c(-c5cccc6oc7ccccc7c56)cccc4c3)n2)cc1]

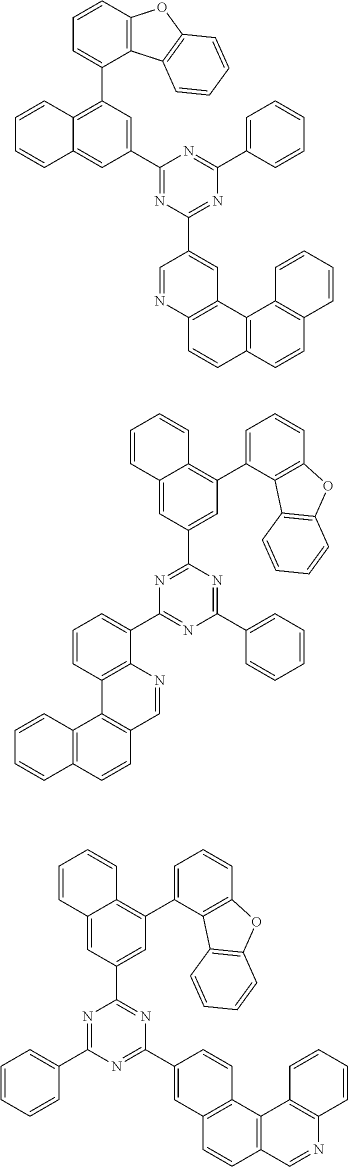

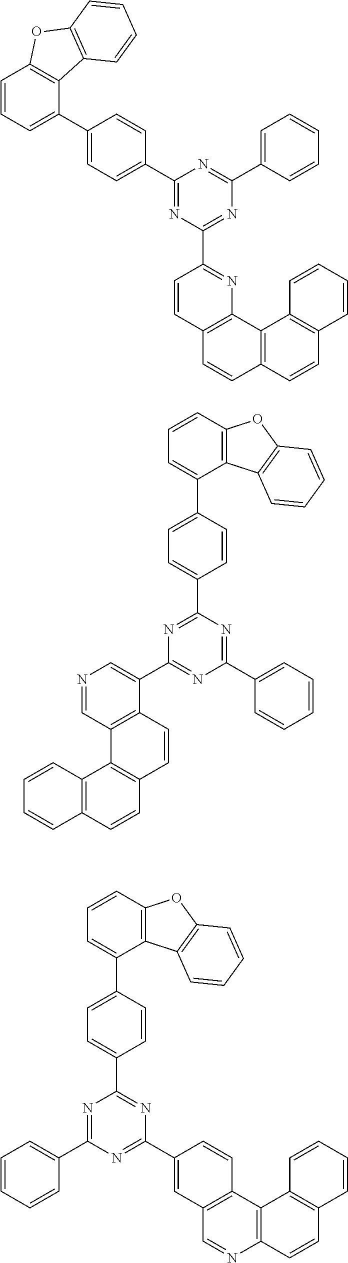

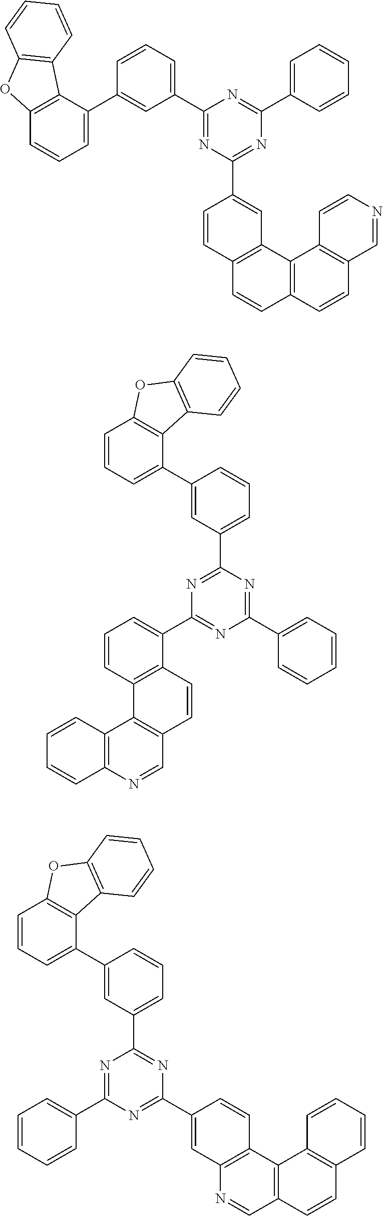 [SMILES: c1ccc(-c2nc(-c3cccc(-c4cccc5oc6ccccc6c45)c3)nc(-c3ccc4ccc5ccc6cnccc6c5c4c3)n2)cc1 ; c1ccc(-c2nc(-c3cccc(-c4cccc5oc6ccccc6c45)c3)nc(-c3cccc4c3ccc3cnc5ccccc5c34)n2)cc1 ; c1ccc(-c2nc(-c3cccc(-c4cccc5oc6ccccc6c45)c3)nc(-c3ccc4c(c3)ncc3ccc5ccccc5c34)n2)cc1]

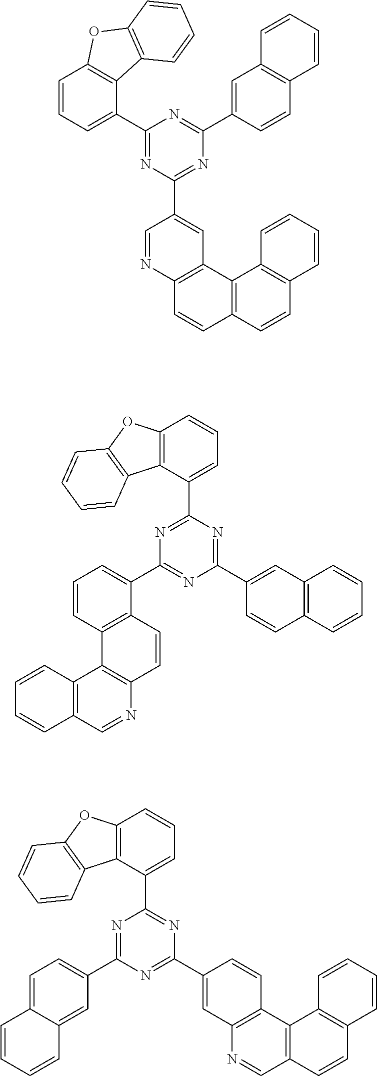 [SMILES: c1ccc2cc(-c3nc(-c4cnc5ccc6ccc7ccccc7c6c5c4)nc(-c4cccc5oc6ccccc6c45)n3)ccc2c1 ; c1ccc2cc(-c3nc(-c4cccc5c4ccc4ncc6ccccc6c45)nc(-c4cccc5oc6ccccc6c45)n3)ccc2c1 ; c1ccc2cc(-c3nc(-c4ccc5c(c4)ncc4ccc6ccccc6c45)nc(-c4cccc5oc6ccccc6c45)n3)ccc2c1]

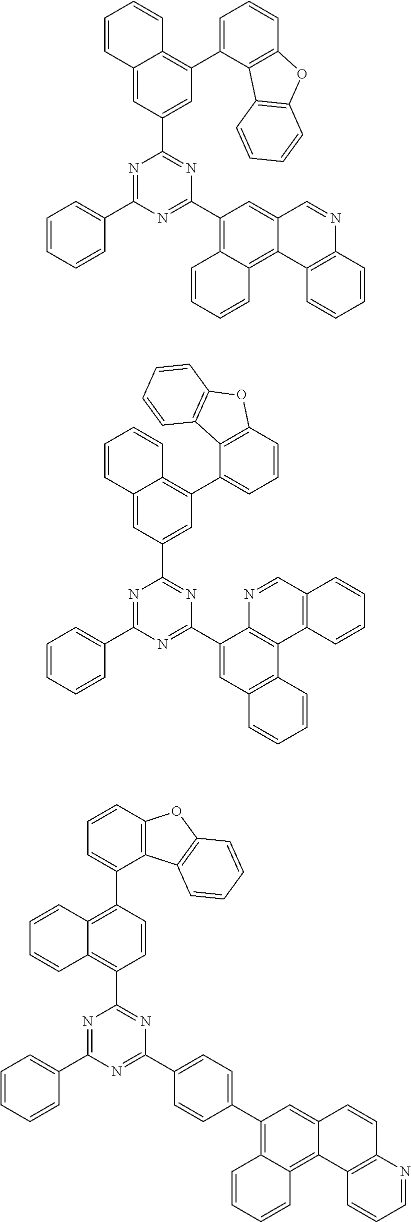 [SMILES: c1ccc(-c2nc(-c3cc(-c4cccc5oc6ccccc6c45)c4ccccc4c3)nc(-c3cc4cnc5ccccc5c4c4ccccc34)n2)cc1 ; c1ccc(-c2nc(-c3cc(-c4cccc5oc6ccccc6c45)c4ccccc4c3)nc(-c3cc4ccccc4c4c3ncc3ccccc34)n2)cc1 ; c1ccc(-c2nc(-c3ccc(-c4cc5ccc6ncccc6c5c5ccccc45)cc3)nc(-c3ccc(-c4cccc5oc6ccccc6c45)c4ccccc34)n2)cc1]

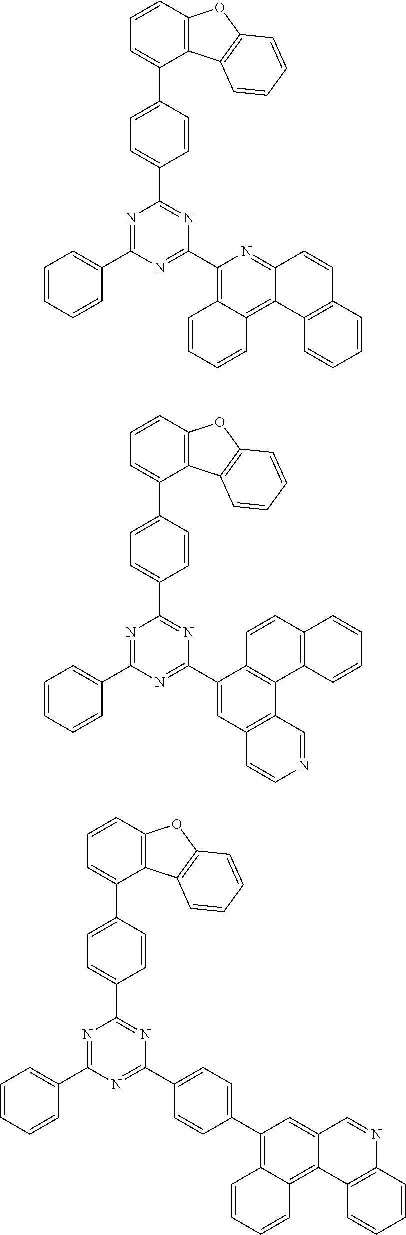 [SMILES: c1ccc(-c2nc(-c3ccc(-c4cccc5oc6ccccc6c45)cc3)nc(-c3nc4ccc5ccccc5c4c4ccccc34)n2)cc1 ; c1ccc(-c2nc(-c3ccc(-c4cccc5oc6ccccc6c45)cc3)nc(-c3cc4ccncc4c4c3ccc3ccccc34)n2)cc1 ; c1ccc(-c2nc(-c3ccc(-c4cc5cnc6ccccc6c5c5ccccc45)cc3)nc(-c3ccc(-c4cccc5oc6ccccc6c45)cc3)n2)cc1]

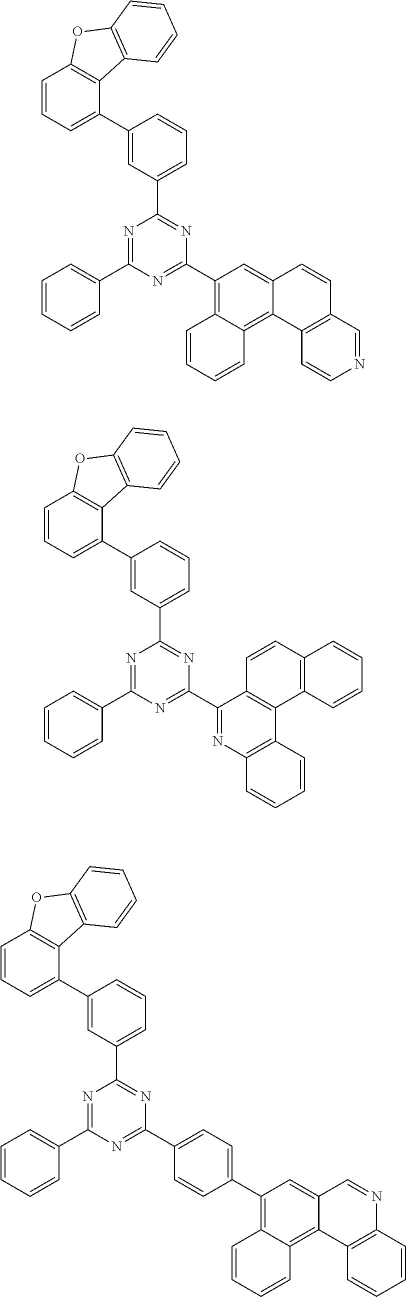 [SMILES: c1ccc(-c2nc(-c3cccc(-c4cccc5oc6ccccc6c45)c3)nc(-c3cc4ccc5cnccc5c4c4ccccc34)n2)cc1 ; c1ccc(-c2nc(-c3cccc(-c4cccc5oc6ccccc6c45)c3)nc(-c3nc4ccccc4c4c3ccc3ccccc34)n2)cc1 ; c1ccc(-c2nc(-c3ccc(-c4cc5cnc6ccccc6c5c5ccccc45)cc3)nc(-c3cccc(-c4cccc5oc6ccccc6c45)c3)n2)cc1]

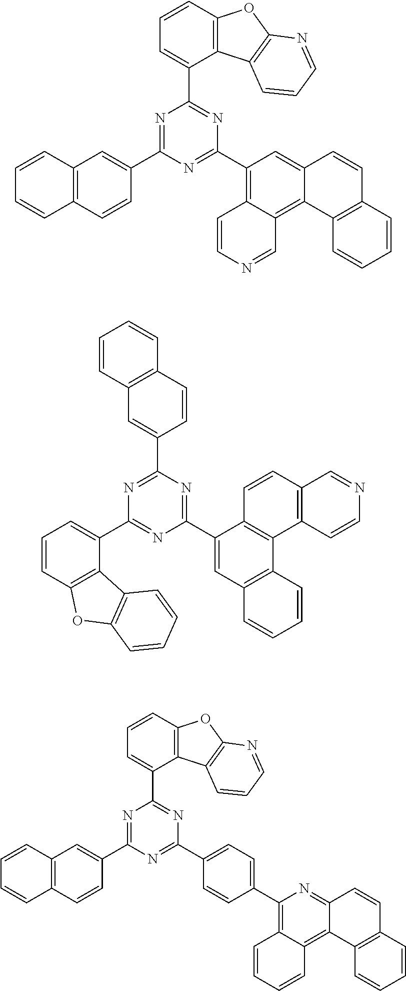 [SMILES: c1ccc2cc(-c3nc(-c4cc5ccc6ccccc6c5c5cnccc45)nc(-c4cccc5oc6ncccc6c45)n3)ccc2c1 ; c1ccc2cc(-c3nc(-c4cc5ccccc5c5c4ccc4cnccc45)nc(-c4cccc5oc6ccccc6c45)n3)ccc2c1 ; c1ccc2cc(-c3nc(-c4ccc(-c5nc6ccc7ccccc7c6c6ccccc56)cc4)nc(-c4cccc5oc6ncccc6c45)n3)ccc2c1]

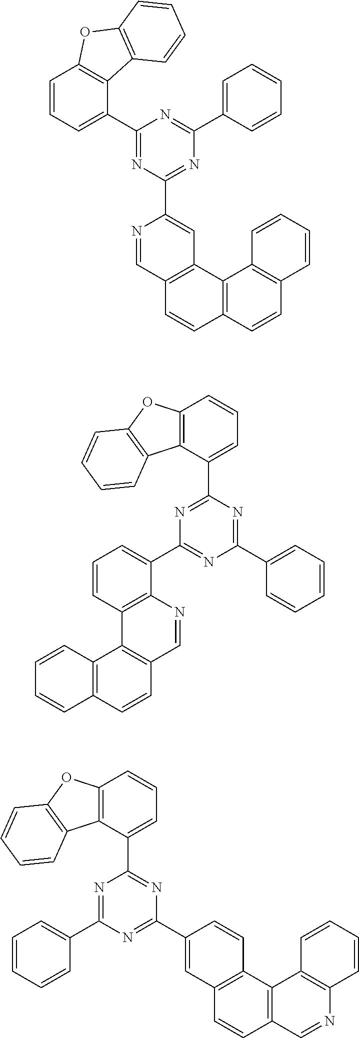 [SMILES: c1ccc(-c2nc(-c3cc4c(ccc5ccc6ccccc6c54)cn3)nc(-c3cccc4oc5ccccc5c34)n2)cc1 ; c1ccc(-c2nc(-c3cccc4c3ncc3ccc5ccccc5c34)nc(-c3cccc4oc5ccccc5c34)n2)cc1 ; c1ccc(-c2nc(-c3ccc4c(ccc5cnc6ccccc6c54)c3)nc(-c3cccc4oc5ccccc5c34)n2)cc1]

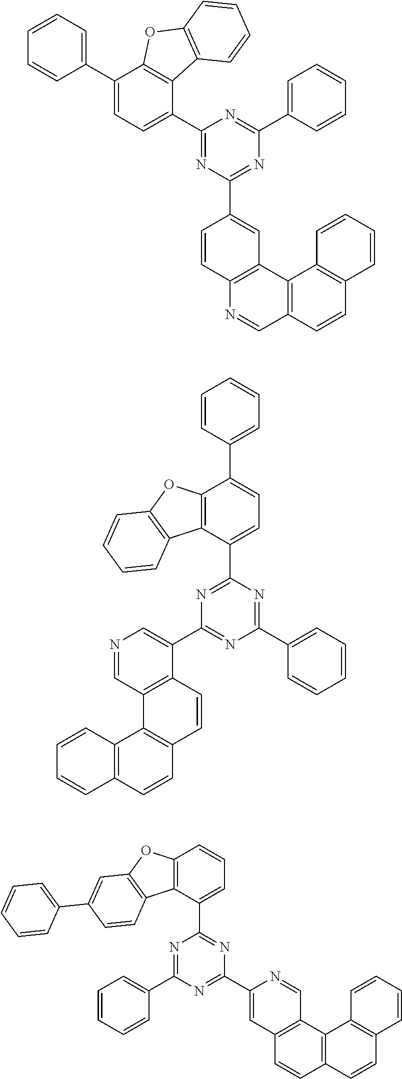 [SMILES: c1ccc(-c2nc(-c3ccc4ncc5ccc6ccccc6c5c4c3)nc(-c3ccc(-c4ccccc4)c4oc5ccccc5c34)n2)cc1 ; c1ccc(-c2nc(-c3cncc4c3ccc3ccc5ccccc5c34)nc(-c3ccc(-c4ccccc4)c4oc5ccccc5c34)n2)cc1 ; c1ccc(-c2ccc3c(c2)oc2cccc(-c4nc(-c5ccccc5)nc(-c5cc6ccc7ccc8ccccc8c7c6cn5)n4)c23)cc1]

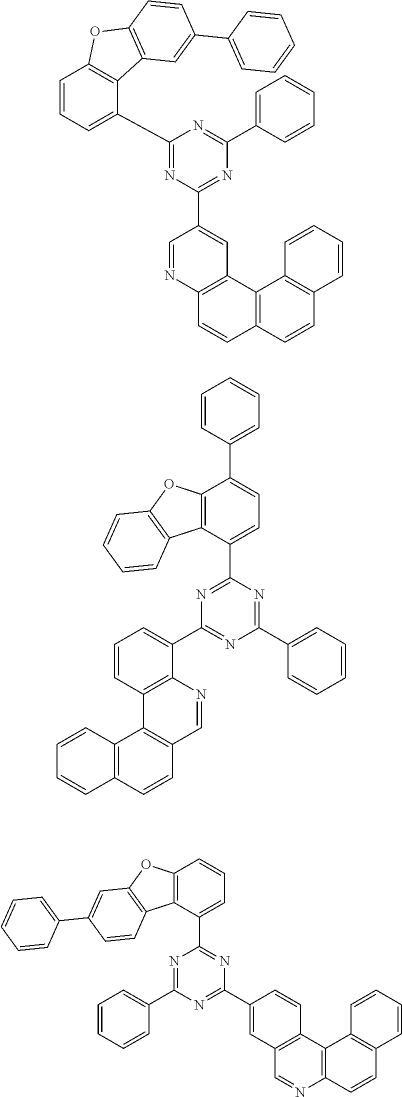

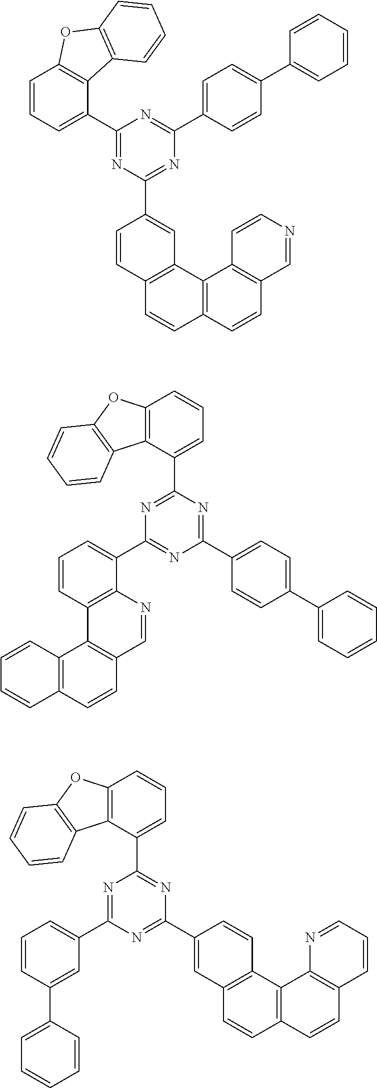 [SMILES: c1ccc(-c2ccc(-c3nc(-c4ccc5ccc6ccc7cnccc7c6c5c4)nc(-c4cccc5oc6ccccc6c45)n3)cc2)cc1 ; c1ccc(-c2ccc(-c3nc(-c4cccc5c4ncc4ccc6ccccc6c45)nc(-c4cccc5oc6ccccc6c45)n3)cc2)cc1 ; c1ccc(-c2cccc(-c3nc(-c4ccc5c(ccc6ccc7cccnc7c65)c4)nc(-c4cccc5oc6ccccc6c45)n3)c2)cc1]

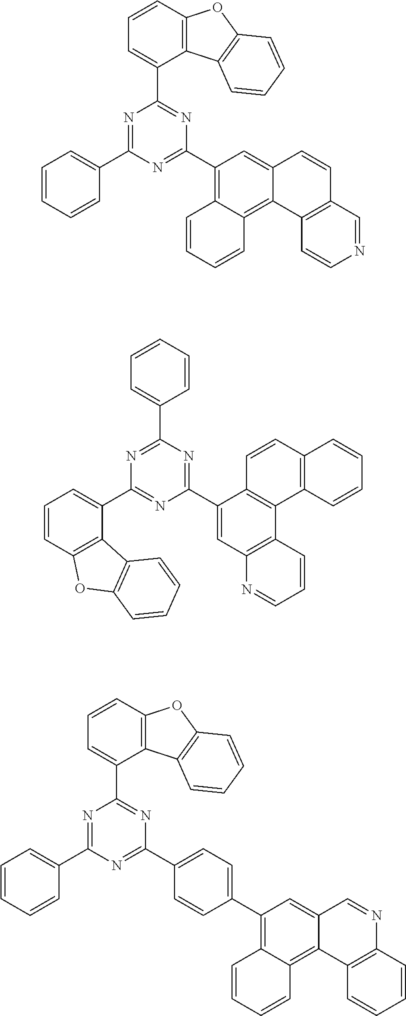 [SMILES: c1ccc(-c2nc(-c3cc4ccc5cnccc5c4c4ccccc34)nc(-c3cccc4oc5ccccc5c34)n2)cc1 ; c1ccc(-c2nc(-c3cc4ncccc4c4c3ccc3ccccc34)nc(-c3cccc4oc5ccccc5c34)n2)cc1 ; c1ccc(-c2nc(-c3ccc(-c4cc5cnc6ccccc6c5c5ccccc45)cc3)nc(-c3cccc4oc5ccccc5c34)n2)cc1]

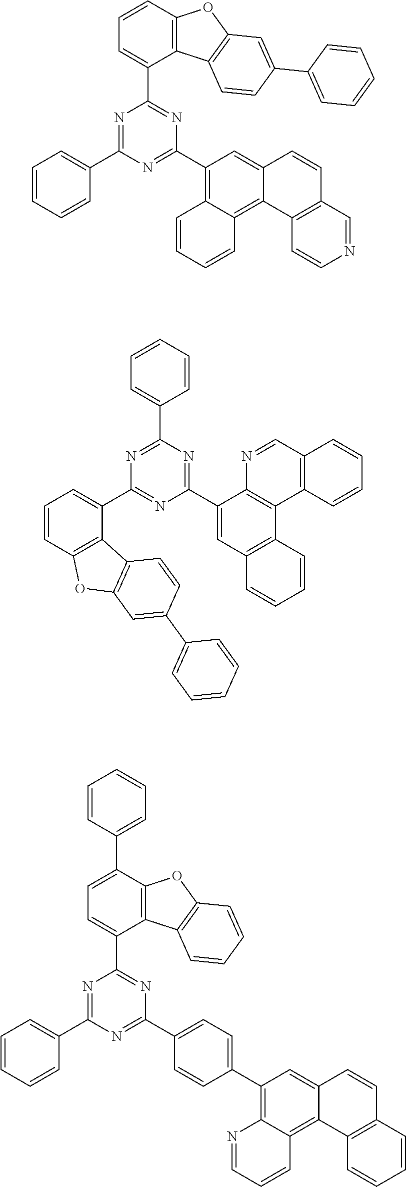 [SMILES: c1ccc(-c2ccc3c(c2)oc2cccc(-c4nc(-c5ccccc5)nc(-c5cc6ccc7cnccc7c6c6ccccc56)n4)c23)cc1 ; c1ccc(-c2ccc3c(c2)oc2cccc(-c4nc(-c5ccccc5)nc(-c5cc6ccccc6c6c5ncc5ccccc56)n4)c23)cc1 ; c1ccc(-c2nc(-c3ccc(-c4cc5ccc6ccccc6c5c5cccnc45)cc3)nc(-c3ccc(-c4ccccc4)c4oc5ccccc5c34)n2)cc1]

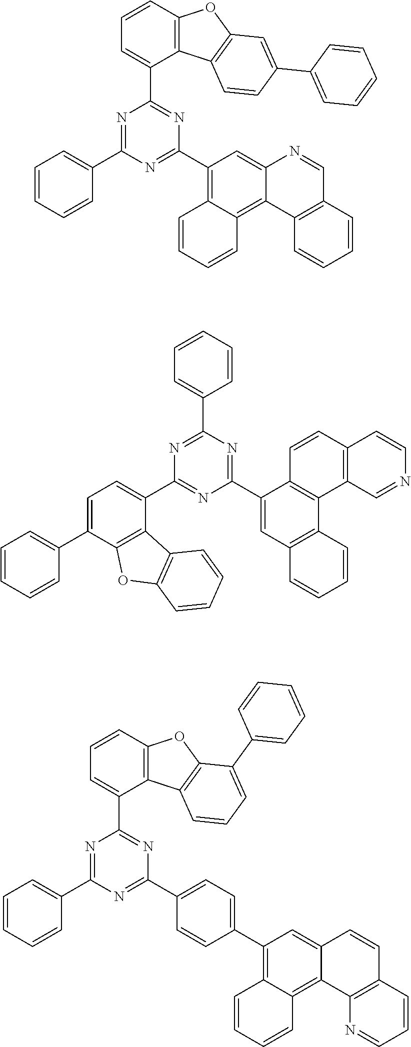 [SMILES: c1ccc(-c2ccc3c(c2)oc2cccc(-c4nc(-c5ccccc5)nc(-c5cc6ncc7ccccc7c6c6ccccc56)n4)c23)cc1 ; c1ccc(-c2nc(-c3cc4ccccc4c4c3ccc3ccncc34)nc(-c3ccc(-c4ccccc4)c4oc5ccccc5c34)n2)cc1 ; c1ccc(-c2nc(-c3ccc(-c4cc5ccc6cccnc6c5c5ccccc45)cc3)nc(-c3cccc4oc5c(-c6ccccc6)cccc5c34)n2)cc1]

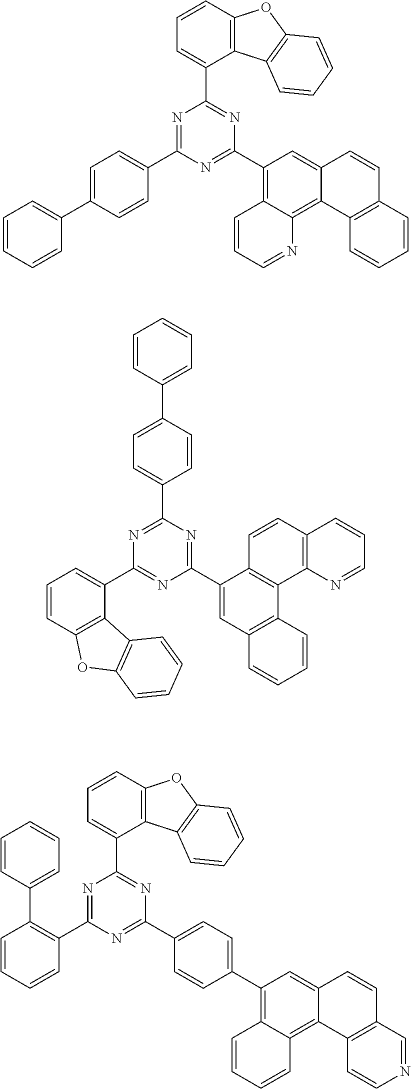 [SMILES: c1ccc(-c2ccc(-c3nc(-c4cc5ccc6ccccc6c5c5ncccc45)nc(-c4cccc5oc6ccccc6c45)n3)cc2)cc1 ; c1ccc(-c2ccc(-c3nc(-c4cc5ccccc5c5c4ccc4cccnc45)nc(-c4cccc5oc6ccccc6c45)n3)cc2)cc1 ; c1ccc(-c2ccccc2-c2nc(-c3ccc(-c4cc5ccc6cnccc6c5c5ccccc45)cc3)nc(-c3cccc4oc5ccccc5c34)n2)cc1]

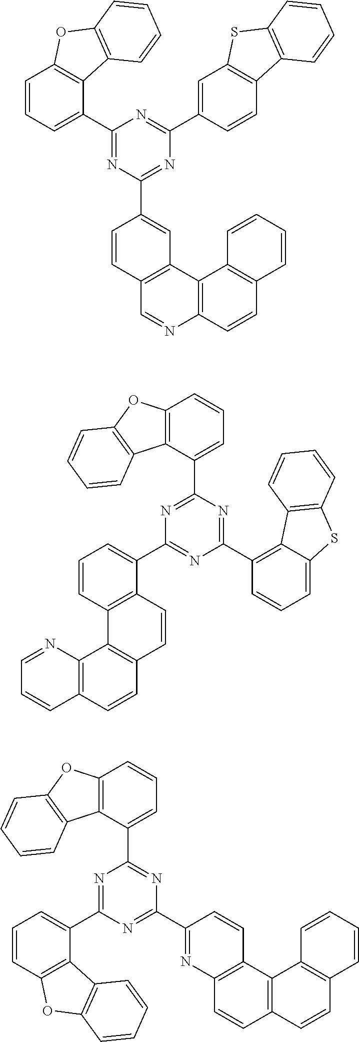 [SMILES: c1ccc2c(c1)ccc1ncc3ccc(-c4nc(-c5ccc6c(c5)sc5ccccc56)nc(-c5cccc6oc7ccccc7c56)n4)cc3c12 ; c1cnc2c(c1)ccc1ccc3c(-c4nc(-c5cccc6oc7ccccc7c56)nc(-c5cccc6sc7ccccc7c56)n4)cccc3c12 ; c1ccc2c(c1)ccc1ccc3nc(-c4nc(-c5cccc6oc7ccccc7c56)nc(-c5cccc6oc7ccccc7c56)n4)ccc3c12]

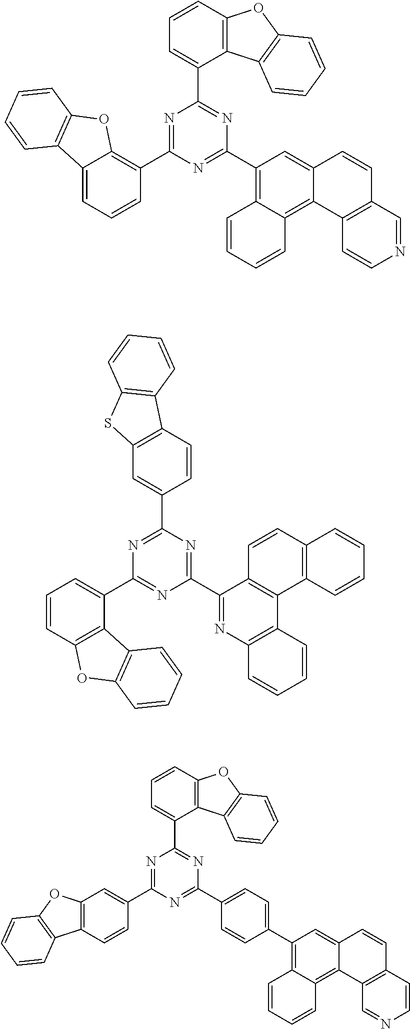 [SMILES: c1ccc2c(c1)oc1c(-c3nc(-c4cc5ccc6cnccc6c5c5ccccc45)nc(-c4cccc5oc6ccccc6c45)n3)cccc12 ; c1ccc2c(c1)ccc1c(-c3nc(-c4ccc5c(c4)sc4ccccc45)nc(-c4cccc5oc6ccccc6c45)n3)nc3ccccc3c12 ; c1ccc2c(c1)oc1cc(-c3nc(-c4ccc(-c5cc6ccc7ccncc7c6c6ccccc56)cc4)nc(-c4cccc5oc6ccccc6c45)n3)ccc12]

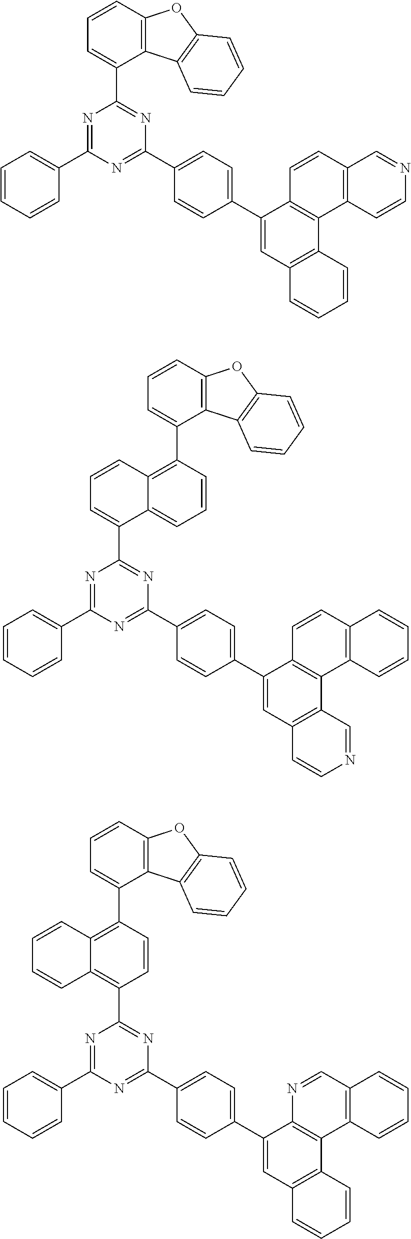 [SMILES: c1ccc(-c2nc(-c3ccc(-c4cc5ccccc5c5c4ccc4cnccc45)cc3)nc(-c3cccc4oc5ccccc5c34)n2)cc1 ; c1ccc(-c2nc(-c3ccc(-c4cc5ccncc5c5c4ccc4ccccc45)cc3)nc(-c3cccc4c(-c5cccc6oc7ccccc7c56)cccc34)n2)cc1 ; c1ccc(-c2nc(-c3ccc(-c4cc5ccccc5c5c4ncc4ccccc45)cc3)nc(-c3ccc(-c4cccc5oc6ccccc6c45)c4ccccc34)n2)cc1]

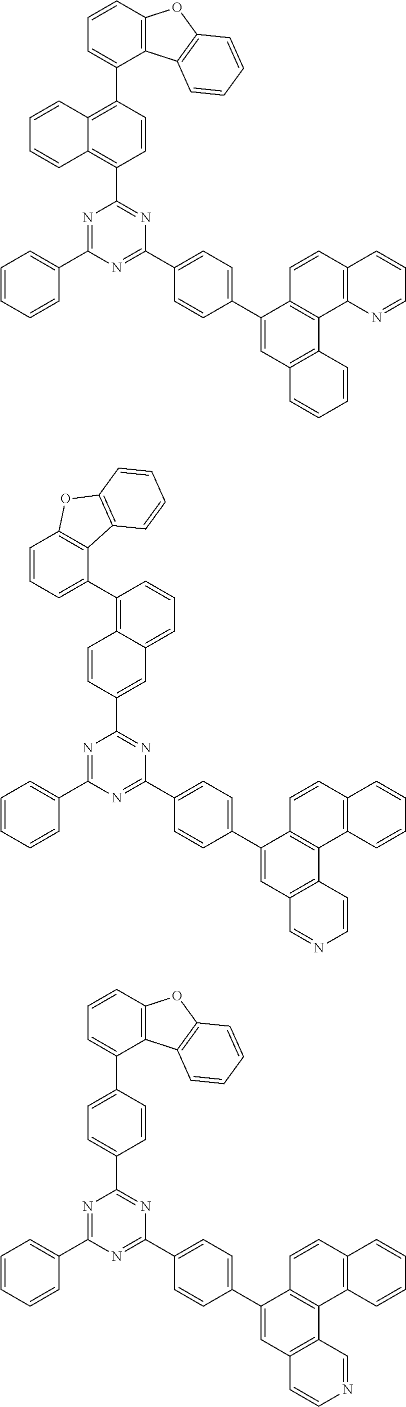 [SMILES: c1ccc(-c2nc(-c3ccc(-c4cc5ccccc5c5c4ccc4cccnc45)cc3)nc(-c3ccc(-c4cccc5oc6ccccc6c45)c4ccccc34)n2)cc1 ; c1ccc(-c2nc(-c3ccc(-c4cc5cnccc5c5c4ccc4ccccc45)cc3)nc(-c3ccc4c(-c5cccc6oc7ccccc7c56)cccc4c3)n2)cc1 ; c1ccc(-c2nc(-c3ccc(-c4cc5ccncc5c5c4ccc4ccccc45)cc3)nc(-c3ccc(-c4cccc5oc6ccccc6c45)cc3)n2)cc1]

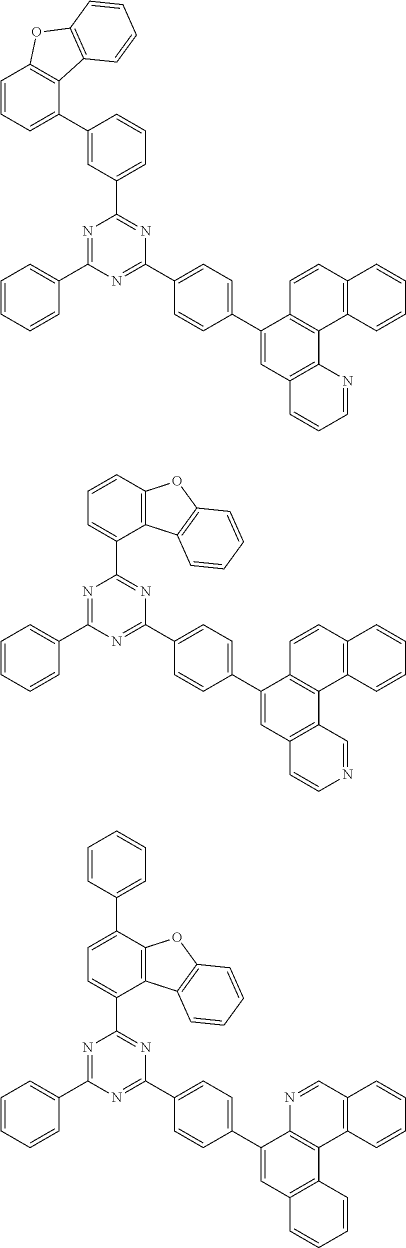 [SMILES: c1ccc(-c2nc(-c3ccc(-c4cc5cccnc5c5c4ccc4ccccc45)cc3)nc(-c3cccc(-c4cccc5oc6ccccc6c45)c3)n2)cc1 ; c1ccc(-c2nc(-c3ccc(-c4cc5ccncc5c5c4ccc4ccccc45)cc3)nc(-c3cccc4oc5ccccc5c34)n2)cc1 ; c1ccc(-c2nc(-c3ccc(-c4cc5ccccc5c5c4ncc4ccccc45)cc3)nc(-c3ccc(-c4ccccc4)c4oc5ccccc5c34)n2)cc1]

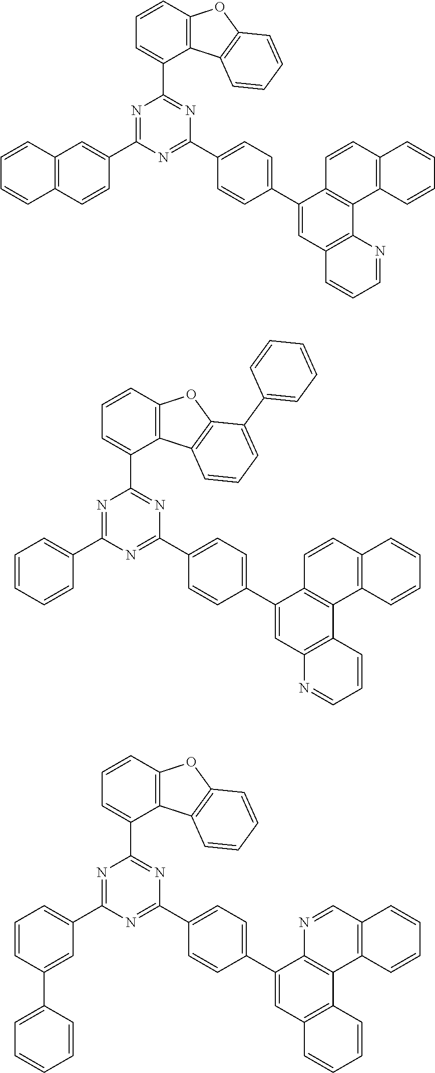 [SMILES: c1ccc2cc(-c3nc(-c4ccc(-c5cc6cccnc6c6c5ccc5ccccc56)cc4)nc(-c4cccc5oc6ccccc6c45)n3)ccc2c1 ; c1ccc(-c2nc(-c3ccc(-c4cc5ncccc5c5c4ccc4ccccc45)cc3)nc(-c3cccc4oc5c(-c6ccccc6)cccc5c34)n2)cc1 ; c1ccc(-c2cccc(-c3nc(-c4ccc(-c5cc6ccccc6c6c5ncc5ccccc56)cc4)nc(-c4cccc5oc6ccccc6c45)n3)c2)cc1]

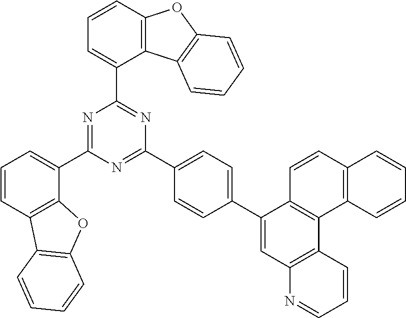 [SMILES: c1ccc2c(c1)ccc1c(-c3ccc(-c4nc(-c5cccc6c5oc5ccccc56)nc(-c5cccc6oc7ccccc7c56)n4)cc3)cc3ncccc3c12]

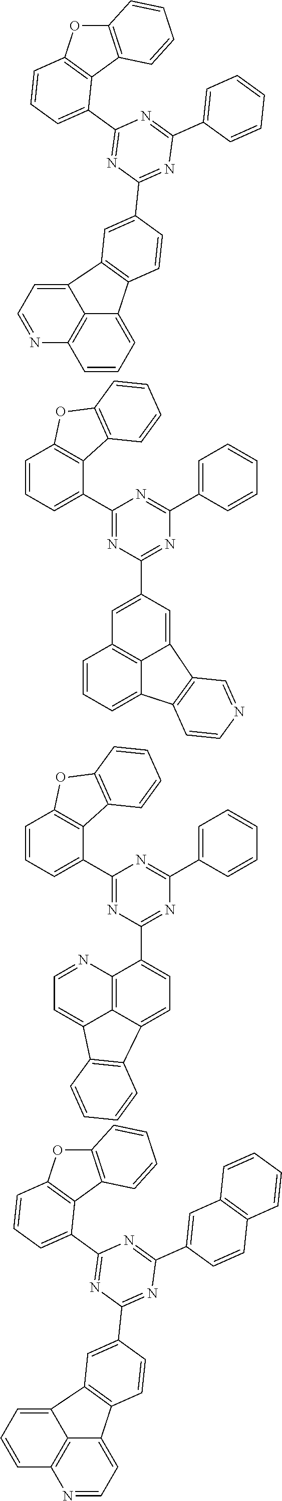

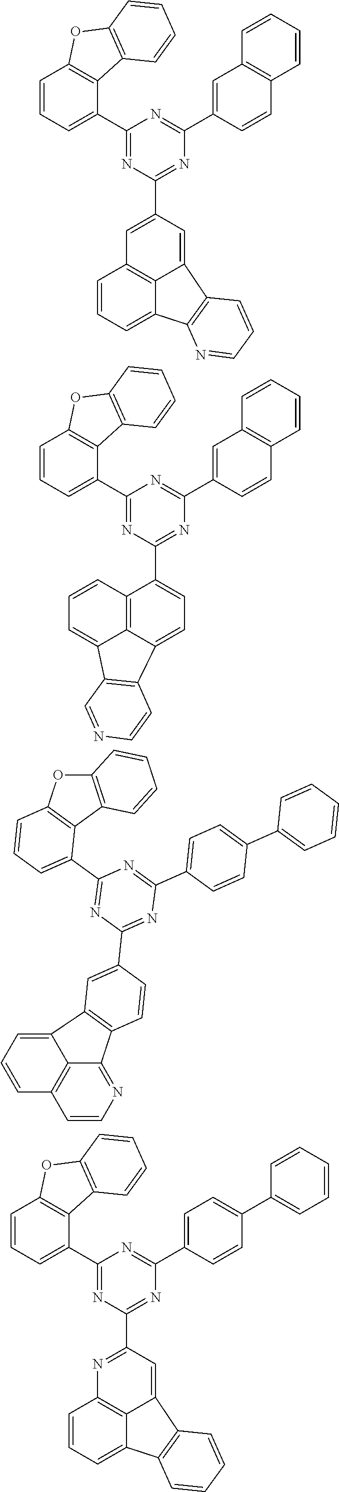

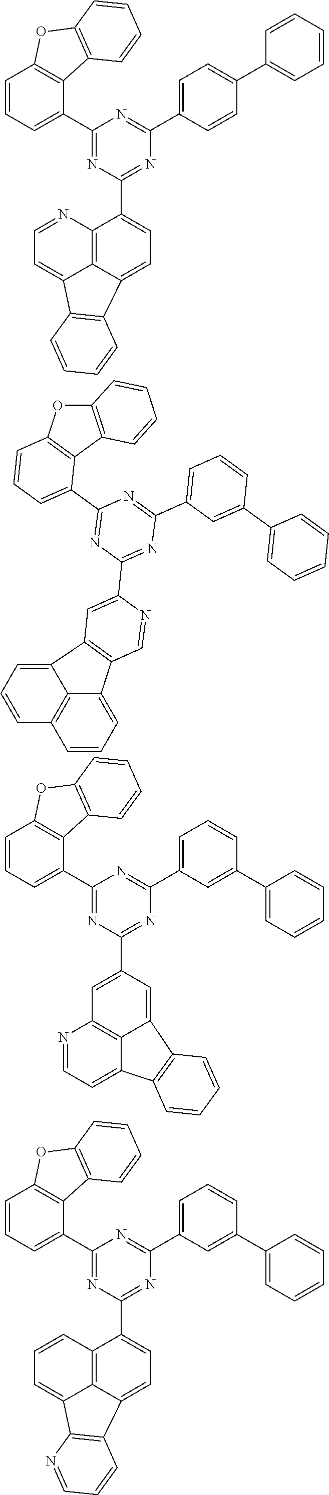

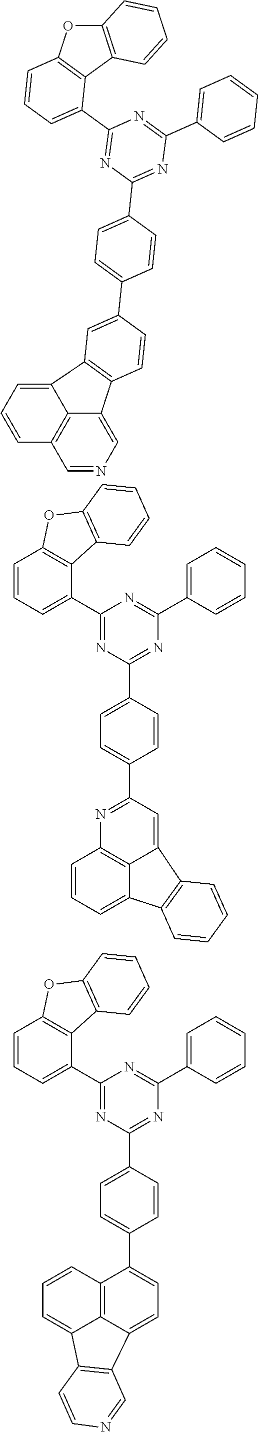

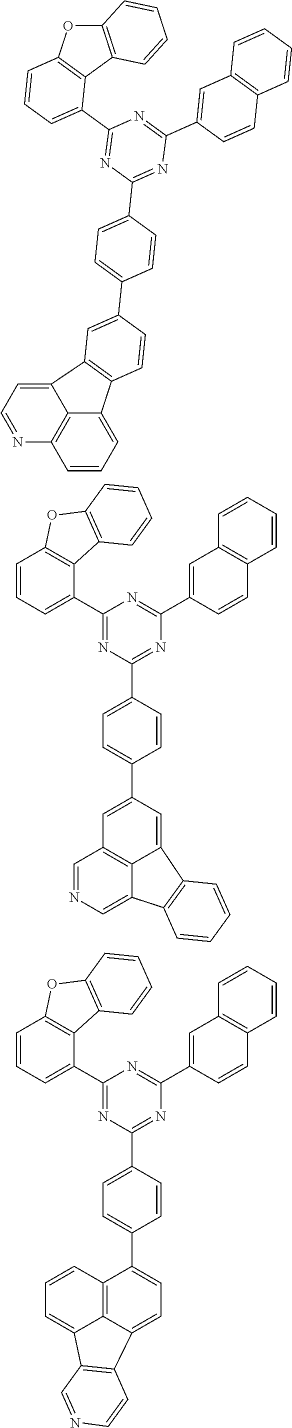

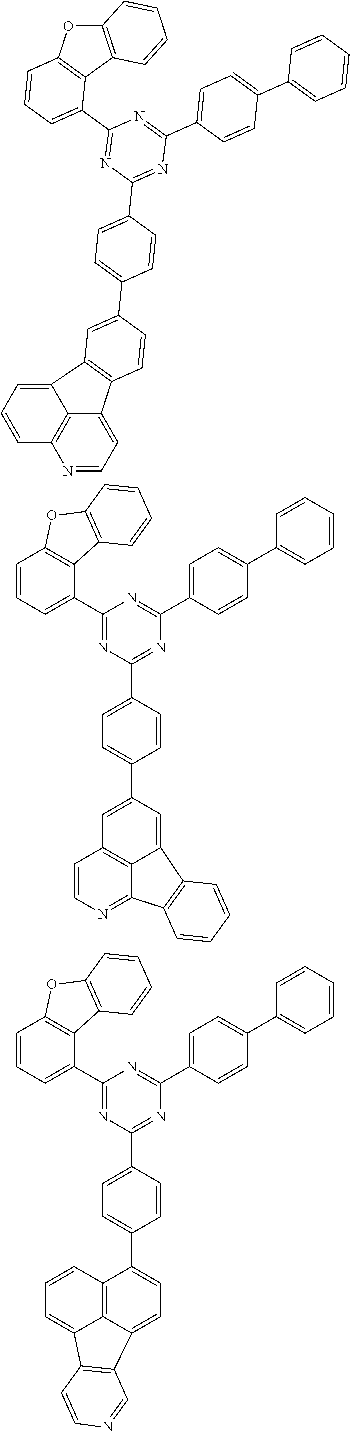

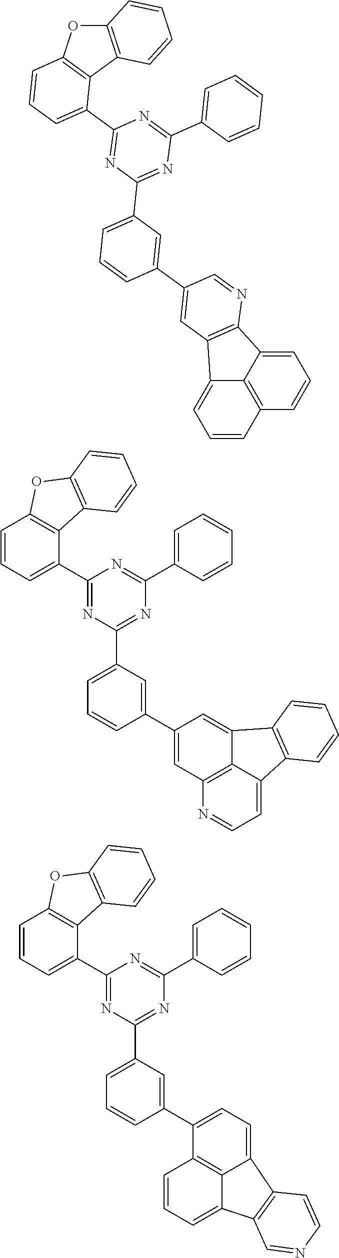 [SMILES: c1ccc(-c2nc(-c3cccc(-c4cnc5c(c4)-c4cccc6cccc-5c46)c3)nc(-c3cccc4oc5ccccc5c34)n2)cc1 ; c1ccc(-c2nc(-c3cccc(-c4cc5c6c(ccnc6c4)-c4ccccc4-5)c3)nc(-c3cccc4oc5ccccc5c34)n2)cc1 ; c1ccc(-c2nc(-c3cccc(-c4ccc5c6c(cccc46)-c4cnccc4-5)c3)nc(-c3cccc4oc5ccccc5c34)n2)cc1]

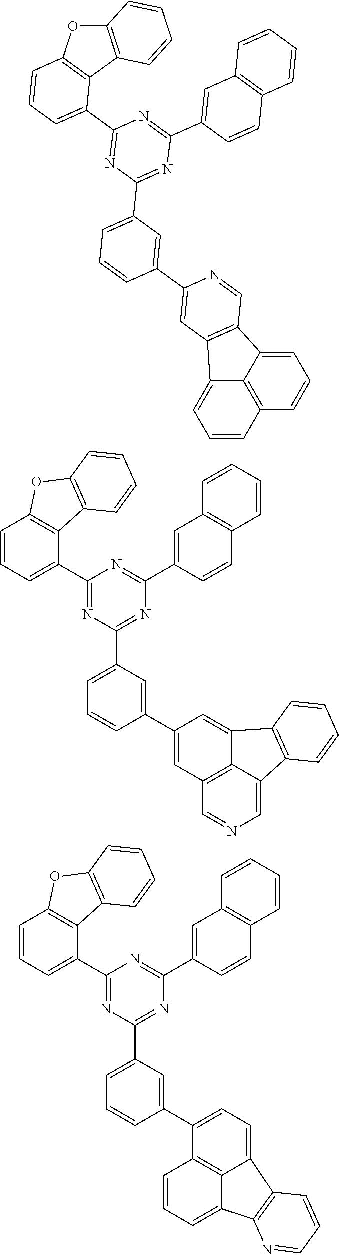

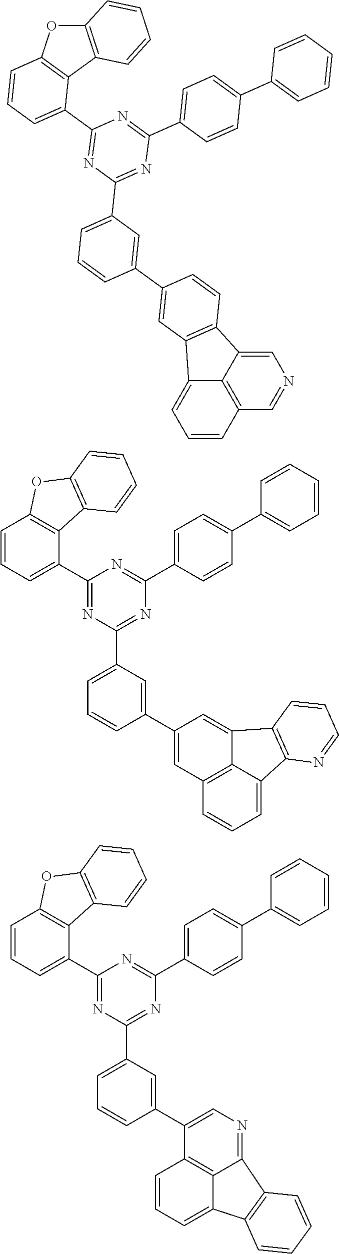 [SMILES: c1ccc(-c2ccc(-c3nc(-c4cccc(-c5ccc6c(c5)-c5cccc7cncc-6c57)c4)nc(-c4cccc5oc6ccccc6c45)n3)cc2)cc1 ; c1ccc(-c2ccc(-c3nc(-c4cccc(-c5cc6c7c(cccc7c5)-c5ncccc5-6)c4)nc(-c4cccc5oc6ccccc6c45)n3)cc2)cc1 ; c1ccc(-c2ccc(-c3nc(-c4cccc(-c5cnc6c7c(cccc57)-c5ccccc5-6)c4)nc(-c4cccc5oc6ccccc6c45)n3)cc2)cc1]

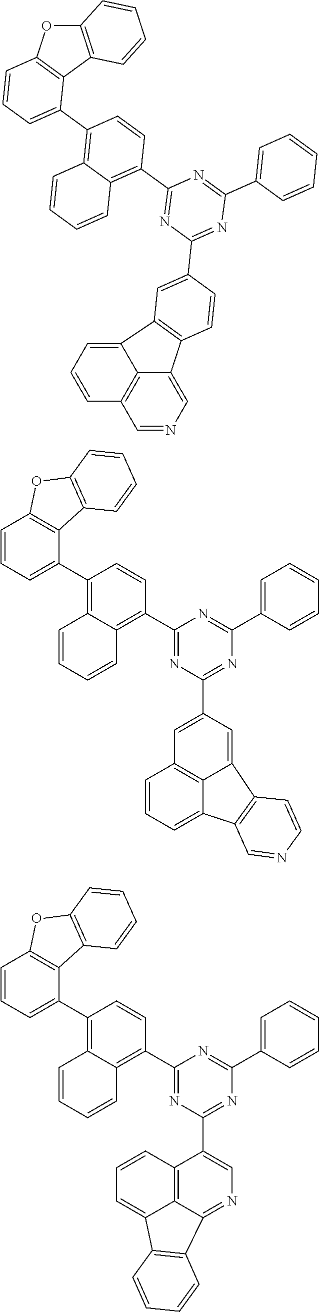

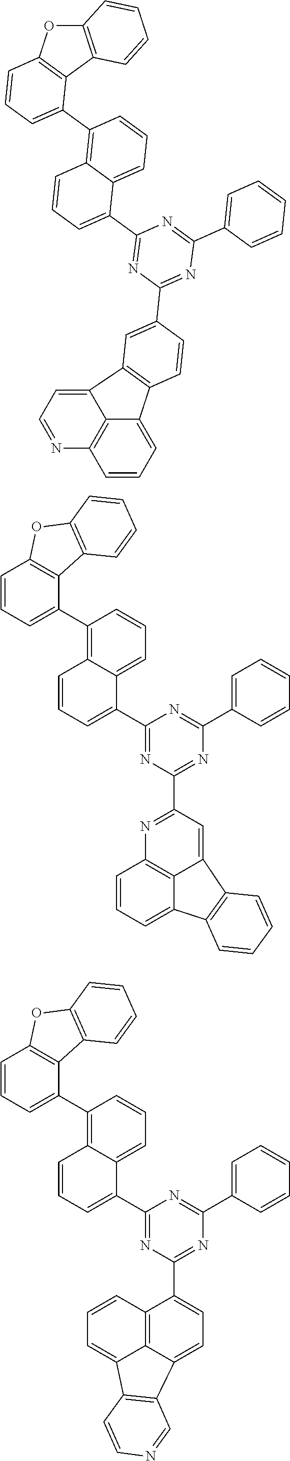

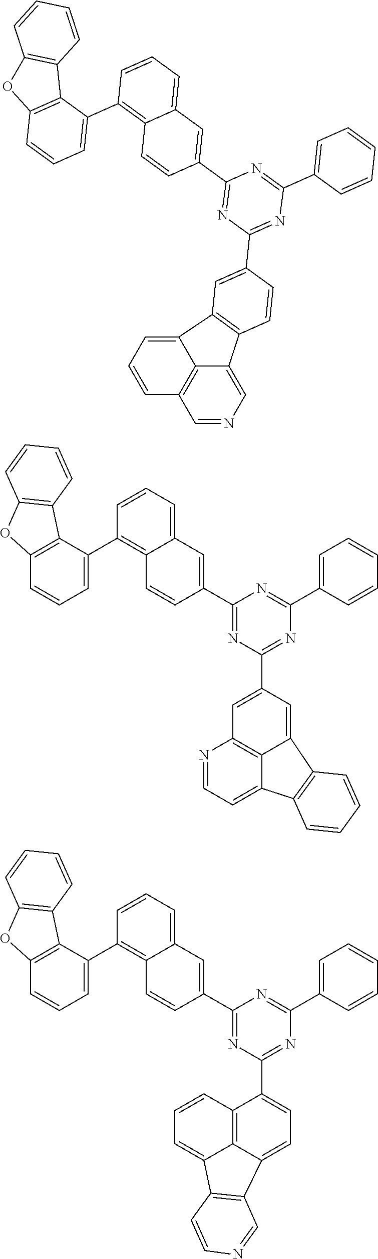 [SMILES: c1ccc(-c2nc(-c3ccc4c(c3)-c3cccc5cncc-4c35)nc(-c3ccc4c(-c5cccc6oc7ccccc7c56)cccc4c3)n2)cc1 ; c1ccc(-c2nc(-c3ccc4c(-c5cccc6oc7ccccc7c56)cccc4c3)nc(-c3cc4c5c(ccnc5c3)-c3ccccc3-4)n2)cc1 ; c1ccc(-c2nc(-c3ccc4c(-c5cccc6oc7ccccc7c56)cccc4c3)nc(-c3ccc4c5c(cccc35)-c3ccncc3-4)n2)cc1]

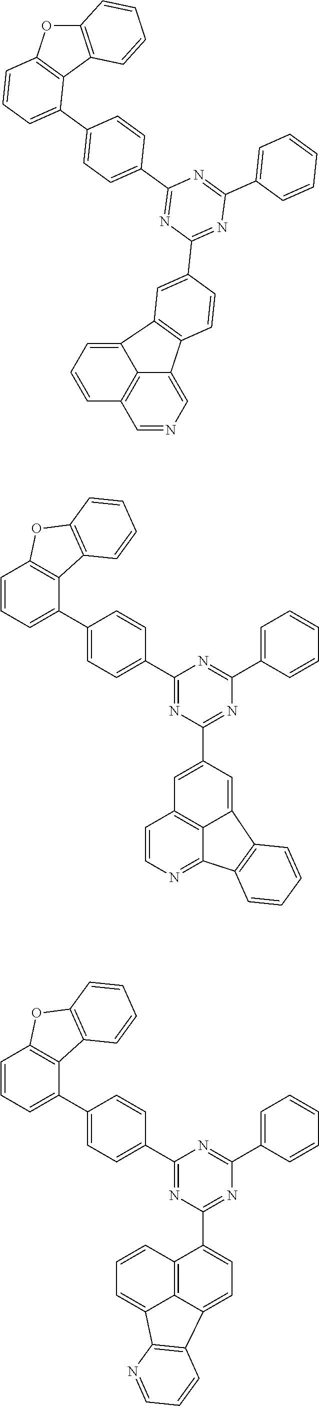 [SMILES: c1ccc(-c2nc(-c3ccc(-c4cccc5oc6ccccc6c45)cc3)nc(-c3ccc4c(c3)-c3cccc5cncc-4c35)n2)cc1 ; c1ccc(-c2nc(-c3ccc(-c4cccc5oc6ccccc6c45)cc3)nc(-c3cc4c5c(nccc5c3)-c3ccccc3-4)n2)cc1 ; c1ccc(-c2nc(-c3ccc(-c4cccc5oc6ccccc6c45)cc3)nc(-c3ccc4c5c(cccc35)-c3ncccc3-4)n2)cc1]

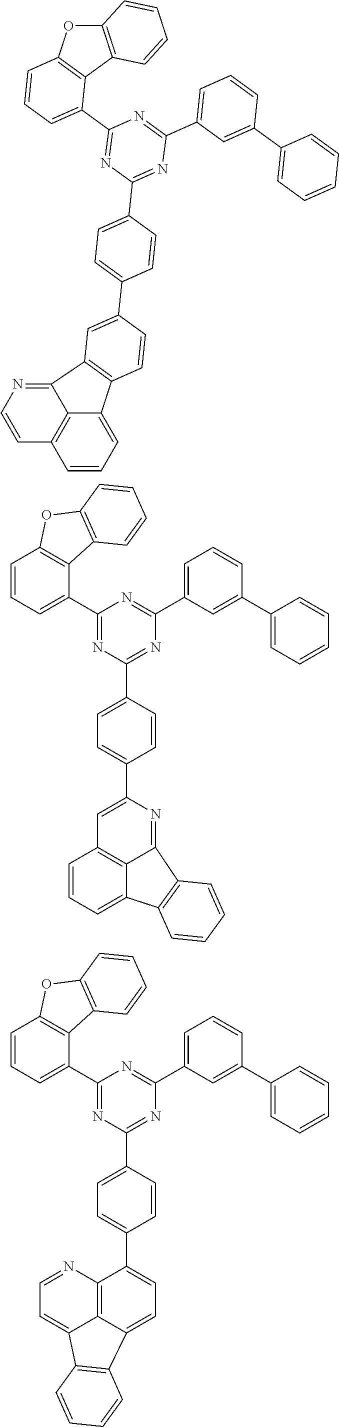

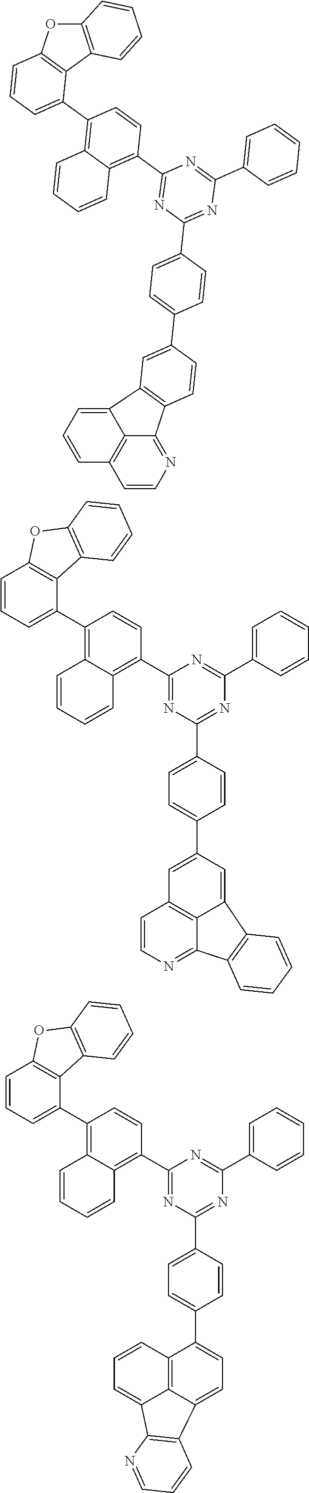

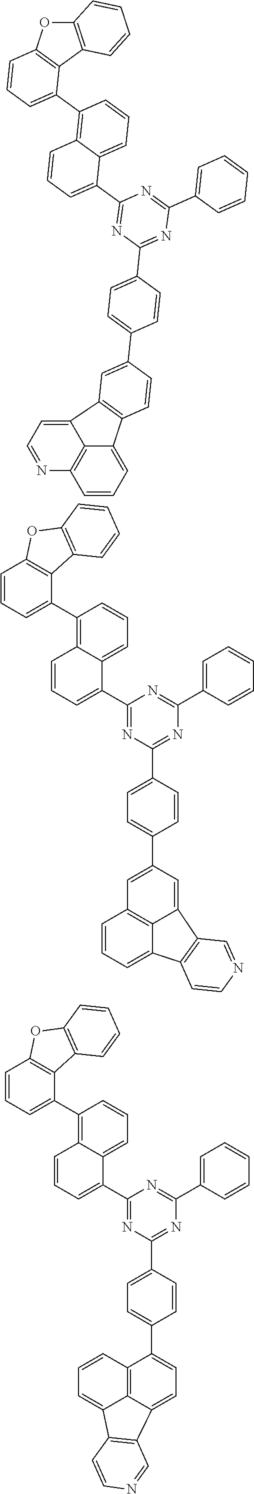

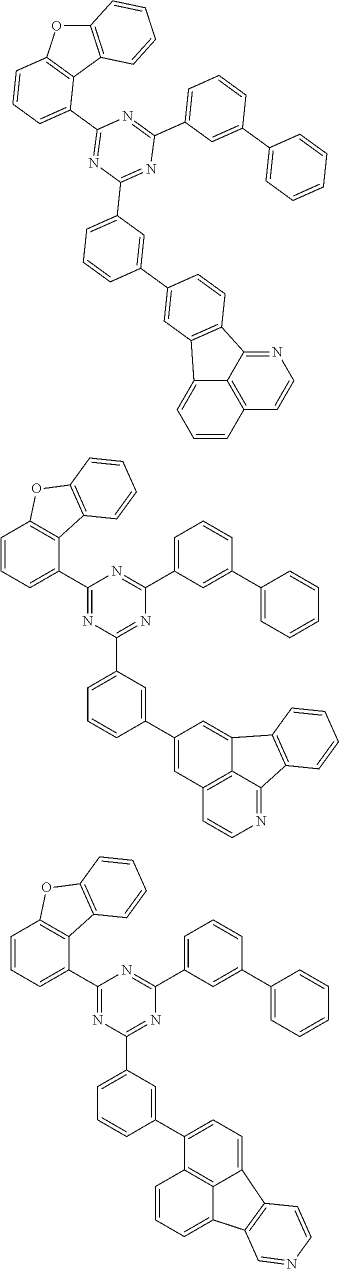 [SMILES: c1ccc(-c2cccc(-c3nc(-c4cccc(-c5ccc6c(c5)-c5cccc7ccnc-6c57)c4)nc(-c4cccc5oc6ccccc6c45)n3)c2)cc1 ; c1ccc(-c2cccc(-c3nc(-c4cccc(-c5cc6c7c(nccc7c5)-c5ccccc5-6)c4)nc(-c4cccc5oc6ccccc6c45)n3)c2)cc1 ; c1ccc(-c2cccc(-c3nc(-c4cccc(-c5ccc6c7c(cccc57)-c5cnccc5-6)c4)nc(-c4cccc5oc6ccccc6c45)n3)c2)cc1]

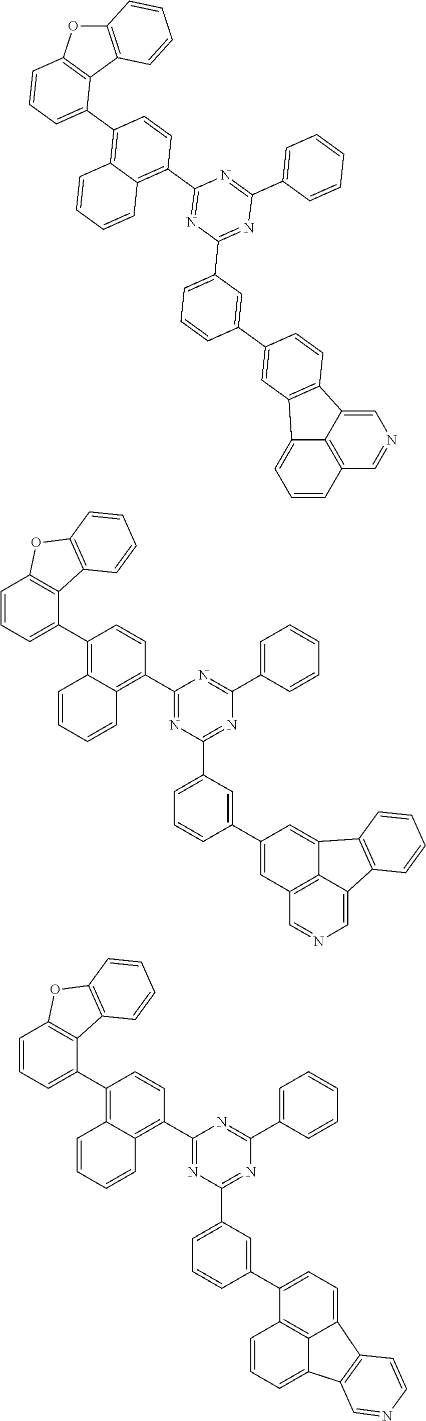 [SMILES: c1ccc(-c2nc(-c3cccc(-c4ccc5c(c4)-c4cccc6cncc-5c46)c3)nc(-c3ccc(-c4cccc5oc6ccccc6c45)c4ccccc34)n2)cc1 ; c1ccc(-c2nc(-c3cccc(-c4cc5c6c(cncc6c4)-c4ccccc4-5)c3)nc(-c3ccc(-c4cccc5oc6ccccc6c45)c4ccccc34)n2)cc1 ; c1ccc(-c2nc(-c3cccc(-c4ccc5c6c(cccc46)-c4cnccc4-5)c3)nc(-c3ccc(-c4cccc5oc6ccccc6c45)c4ccccc34)n2)cc1]

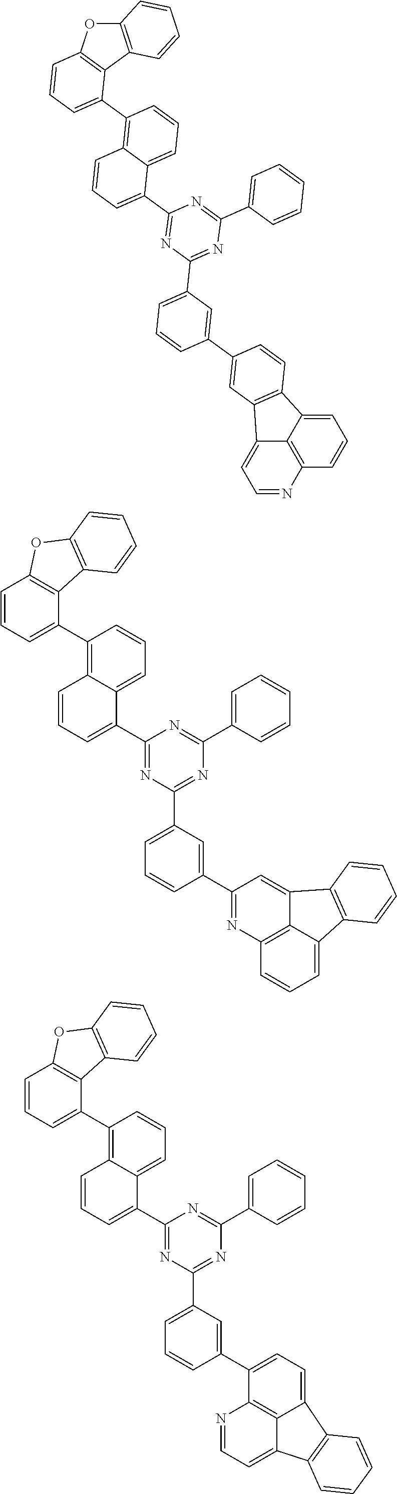 [SMILES: c1ccc(-c2nc(-c3cccc(-c4ccc5c(c4)-c4ccnc6cccc-5c46)c3)nc(-c3cccc4c(-c5cccc6oc7ccccc7c56)cccc34)n2)cc1 ; c1ccc(-c2nc(-c3cccc(-c4cc5c6c(cccc6n4)-c4ccccc4-5)c3)nc(-c3cccc4c(-c5cccc6oc7ccccc7c56)cccc34)n2)cc1 ; c1ccc(-c2nc(-c3cccc(-c4ccc5c6c(ccnc46)-c4ccccc4-5)c3)nc(-c3cccc4c(-c5cccc6oc7ccccc7c56)cccc34)n2)cc1]

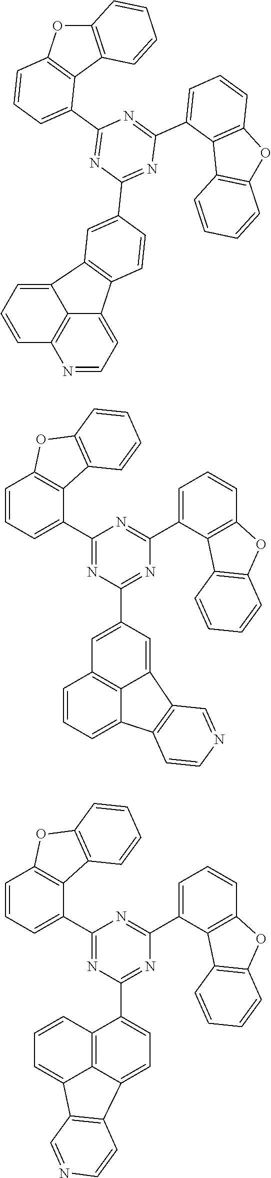

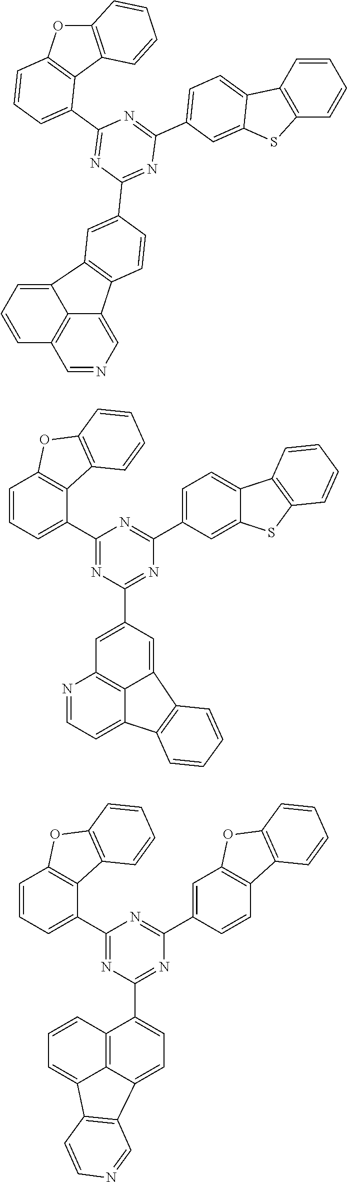

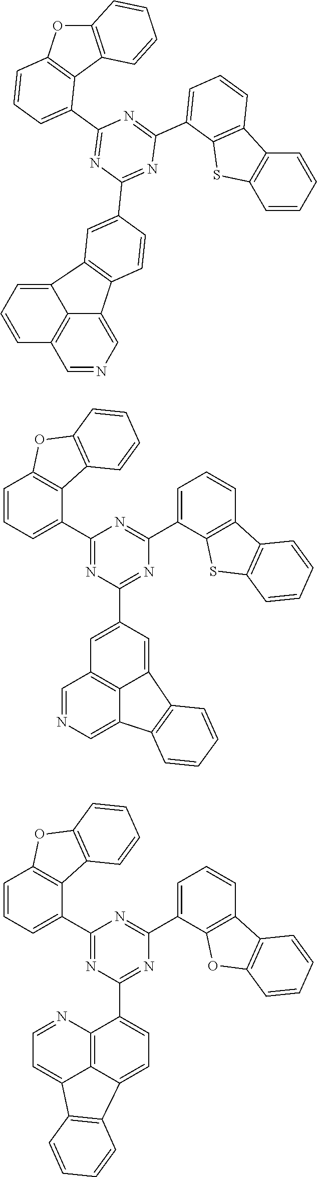

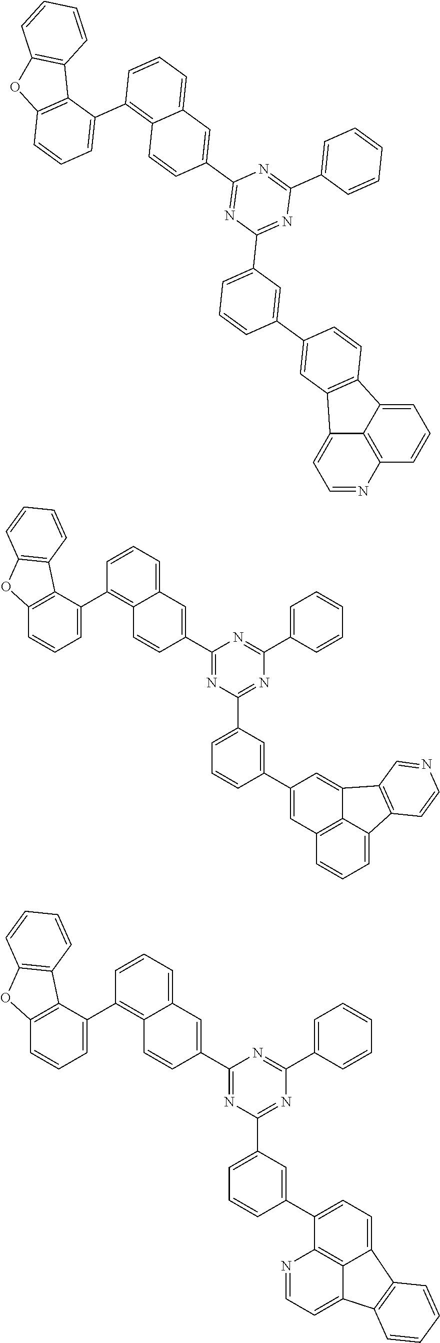 [SMILES: c1ccc(-c2nc(-c3cccc(-c4ccc5c(c4)-c4ccnc6cccc-5c46)c3)nc(-c3ccc4c(-c5cccc6oc7ccccc7c56)cccc4c3)n2)cc1 ; c1ccc(-c2nc(-c3cccc(-c4cc5c6c(cccc6c4)-c4ccncc4-5)c3)nc(-c3ccc4c(-c5cccc6oc7ccccc7c56)cccc4c3)n2)cc1 ; c1ccc(-c2nc(-c3cccc(-c4ccc5c6c(ccnc46)-c4ccccc4-5)c3)nc(-c3ccc4c(-c5cccc6oc7ccccc7c56)cccc4c3)n2)cc1]

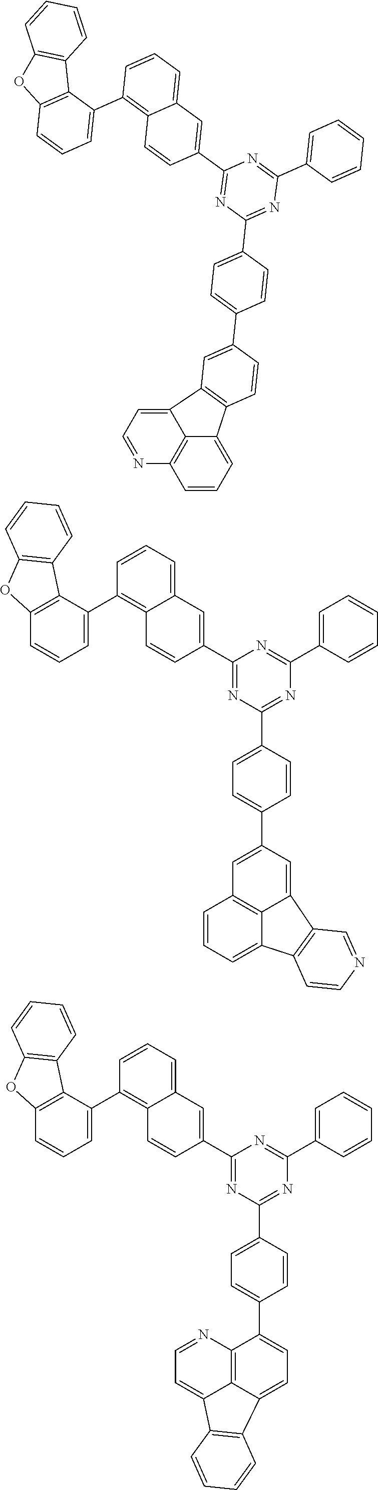 [SMILES: c1ccc(-c2nc(-c3ccc(-c4ccc5c(c4)-c4ccnc6cccc-5c46)cc3)nc(-c3ccc4c(-c5cccc6oc7ccccc7c56)cccc4c3)n2)cc1 ; c1ccc(-c2nc(-c3ccc(-c4cc5c6c(cccc6c4)-c4ccncc4-5)cc3)nc(-c3ccc4c(-c5cccc6oc7ccccc7c56)cccc4c3)n2)cc1 ; c1ccc(-c2nc(-c3ccc(-c4ccc5c6c(ccnc46)-c4ccccc4-5)cc3)nc(-c3ccc4c(-c5cccc6oc7ccccc7c56)cccc4c3)n2)cc1]

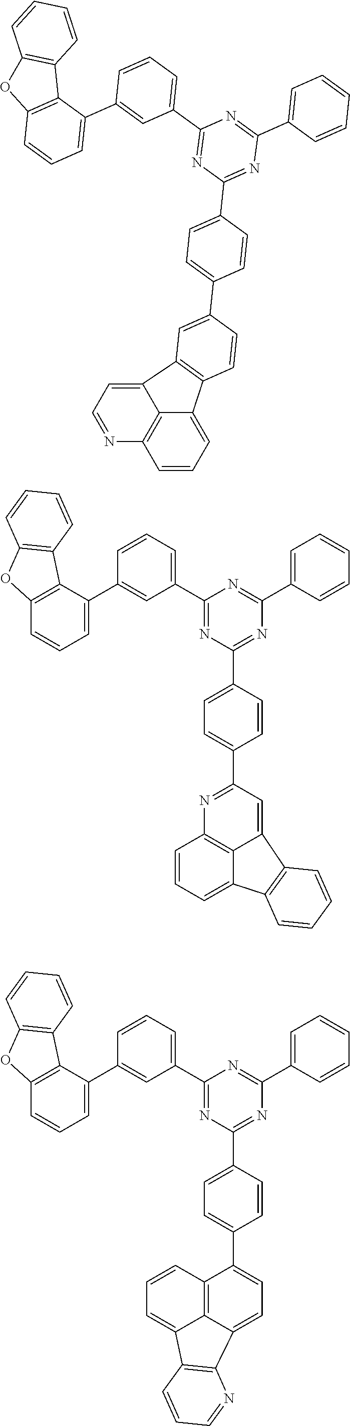 [SMILES: c1ccc(-c2nc(-c3ccc(-c4ccc5c(c4)-c4ccnc6cccc-5c46)cc3)nc(-c3cccc(-c4cccc5oc6ccccc6c45)c3)n2)cc1 ; c1ccc(-c2nc(-c3ccc(-c4cc5c6c(cccc6n4)-c4ccccc4-5)cc3)nc(-c3cccc(-c4cccc5oc6ccccc6c45)c3)n2)cc1 ; c1ccc(-c2nc(-c3ccc(-c4ccc5c6c(cccc46)-c4cccnc4-5)cc3)nc(-c3cccc(-c4cccc5oc6ccccc6c45)c3)n2)cc1]

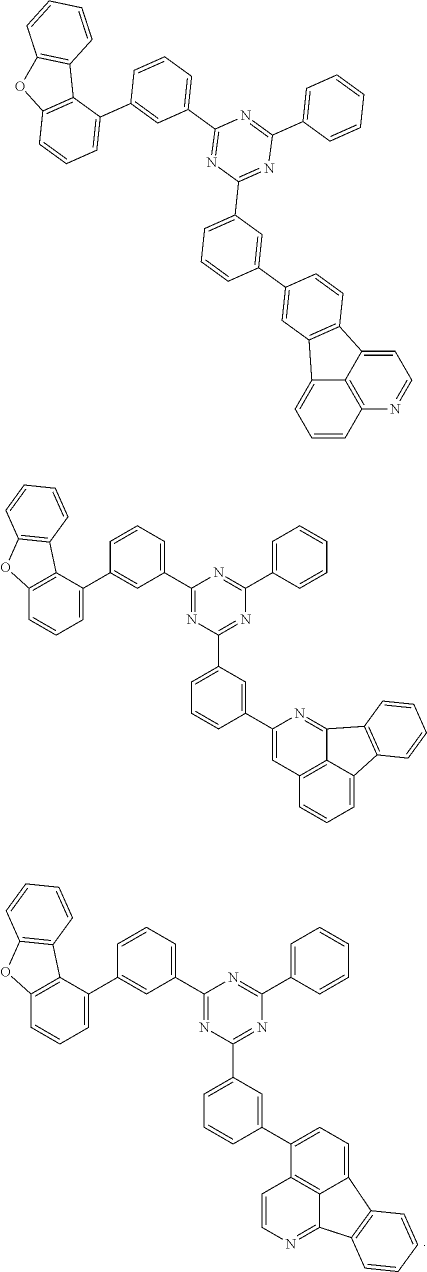 [SMILES: c1ccc(-c2nc(-c3cccc(-c4ccc5c(c4)-c4cccc6nccc-5c46)c3)nc(-c3cccc(-c4cccc5oc6ccccc6c45)c3)n2)cc1 ; c1ccc(-c2nc(-c3cccc(-c4cc5cccc6c5c(n4)-c4ccccc4-6)c3)nc(-c3cccc(-c4cccc5oc6ccccc6c45)c3)n2)cc1 ; c1ccc(-c2nc(-c3cccc(-c4ccc5c6c(nccc46)-c4ccccc4-5)c3)nc(-c3cccc(-c4cccc5oc6ccccc6c45)c3)n2)cc1]

10. An organic light emitting device, comprising:
a first electrode;
a second electrode that is opposite to the first electrode; and
one or more organic material layers that are between the first electrode and the second electrode, wherein one or more layers of the one or more organic material layers comprises the compound of claim 1.

11. The organic light emitting device according to claim 10, wherein:
the organic material layer comprising the compound is a light emitting layer.
